(19) United States
(12) Patent Application Publication
(10) Pub. No.: US 2026/0284244 A1
(43) Pub. Date: Sep. 24, 2026

(54) Humanised antibodies and uses thereof

(71) Applicant: Telix Pharmaceuticals (Innovations) Pty Ltd, Victoria (AU)

(72) Inventors: Michael Paul WHEATCROFT, Victoria (AU); David CHIN, Victoria (AU); Alesia IVASHKEVICH, Victoria (AU); Alexander Hugo STAUDACHER, Victoria (AU); Michael Paul BROWN, Victoria (AU)

(21) Appl. No.: 19/475,035

(22) PCT Filed: Jul. 12, 2024
(86) PCT No.: PCT/AU2024/050749

§ 371 Date: Oct. 13, 2025

(30) Foreign Application Priority Data
Jul. 13, 2023 (AU) 2023902248

(51) Int. Cl. A61K 51/10; A61K 47/68; C07K 16/18

(52) U.S. Cl. CPC A61K 51/1093; A61K 47/6843; A61K 51/1018; C07K 16/18; A61K 2123/00; C07K 2317/24; C07K 2317/524; C07K 2317/526; C07K 2317/565; C07K 2317/567; C07K 2317/71; C07K 2317/732; C07K 2317/734; C07K 2317/94

(57) ABSTRACT

The invention relates to humanised antibodies for binding to the La/SSB antigen, antigen binding fragments derived therefrom, compositions comprising the antibodies or antigen binding fragments thereof, and uses thereof in methods of treatment.

### FIELD OF THE INVENTION

The present invention relates to humanised antibodies for binding to the La/SSB antigen, antigen binding fragments derived therefrom, compositions comprising the antibodies or antigen binding fragments thereof, and uses thereof in methods of treatment.

### RELATED APPLICATION

This application claims the benefit of priority from Australian provisional application no. 2023902248, the entire content of which is hereby incorporated by reference.

### BACKGROUND OF THE INVENTION

The human Lupus antigen was first described as an autoantigen identified in lupus patients. It is also known by the alternative (Human Genome Organisation; HUGO) name SS-B (Sjögren syndrome antigen B) or abbreviated to La, LA/SSB or hLA.

More recently, the nuclear La protein has been described as a marker of cell death, becoming exposed upon apoptosis of cells. More specifically, the nuclear antigen La is released by injured or dying cells and can bind to the surface of neighbouring intact cells. In the context of tumour therapy, the nuclear antigen La is released by apoptotic tumour cells and binds to the surface of neighbouring tumour cells. Therefore, the La protein has been proposed as an inducible universal surface target and agents for binding to the La protein have been proposed to measure treatment-induced cell death.

There is a need for new agents for use in detecting La antigen. There is also a need for new agents for binding La for use in therapeutic and diagnostic applications.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present invention relates to antigen binding proteins comprising an antigen binding domain for binding to the Lupus antigen (La). In particular, the invention relates to humanised anti-La antibodies and La-binding fragments thereof. The invention also relates to humanised anti-La antibodies comprising one or more substitutions in the constant region for reducing affinity of the antibody for the FcRn, optionally wherein the antibodies are conjugated to a radionuclide.

In a first aspect, the invention provides an antigen binding protein for binding to La, the antigen binding protein comprising an antigen binding domain comprising:

$$FR 1 - CFR 1 - FR 2 - CDR 2 - FR 3 - CDR 3 - FR 4 , and$$

$$FR 1 a - CDR 1 a - FDR 2 a - CDR 2 a - FR 3 a - CDR 3 a - FR 4 a ,$$

wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions;
wherein the antigen binding domain comprises a CDR1, CDR2 and a CDR3 of a heavy variable chain as defined in Table 2 below, and/or a CDR1a, CDR2a and a CDR3a of a variable light chain as defined in in Table 2 below.

In any embodiment, CDR1, CDR2 and CDR3 refer to complementarity determining regions from the variable heavy chain of an antibody (a VH), CDR1a, CDR2a and CDR3a are complementarity determining regions from the variable light chain of an antibody (a VL), or where CDR1, CDR2 and CDR3 are complementarity determining regions from the VL, CDR1a, CDR2a and CDR3a are complementarity determining regions from VH. In such examples, the CDRs may be referred to as CDRH1, CDRH2, CDRH3, CDRL1, CDRL2 and CDRL3 as the case may be.

Reference herein to a protein or antibody that “binds to” La (or La/SSB) provides literal support for a protein or antibody that “binds specifically to” or “specifically binds to” a La (La/SSB).

In preferred embodiments, an antigen binding protein as described herein is capable of binding to or specifically binding to residues 83-107 of La/SSB (as set forth in SEQ ID NO: 96: SEDKTKIRRSPSKPLPEVTDEYKND). In further embodiments, an antigen binding protein of the invention is capable of binding to or specifically binding to residues 92-107 of La/SSB (as set forth in SEQ ID NO: 97: SPSKPLPEVTDEYKND).

In any embodiment, the invention provides an antigen binding protein for binding to La, wherein the antigen binding protein competitively inhibits the binding to La of an antibody:
comprising a VH comprising a sequence as set forth in SEQ ID NO: 1 and a VL comprising a sequence as set forth in SEQ ID NO: 5;
comprising a VH comprising a sequence as set forth in SEQ ID NO: 1 and a VL comprising a sequence as set forth in SEQ ID NO: 6;
comprising a VH comprising a sequence as set forth in SEQ ID NO: 2 and a VL comprising a sequence as set forth in SEQ ID NO: 4;
comprising a VH comprising a sequence as set forth in SEQ ID NO: 2 and a VL comprising a sequence as set forth in SEQ ID NO: 5;
comprising a VH comprising a sequence as set forth in SEQ ID NO: 2 and a VL comprising a sequence as set forth in SEQ ID NO: 6;
comprising a VH comprising a sequence as set forth in SEQ ID NO: 3 and a VL comprising a sequence as set forth in SEQ ID NO: 4;
comprising a VH comprising a sequence as set forth in SEQ ID NO: 3 and a VL comprising a sequence as set forth in SEQ ID NO: 5;
comprising a VH comprising a sequence as set forth in SEQ ID NO: 3 and a VL comprising a sequence as set forth in SEQ ID NO: 6;
In any embodiment, the invention provides an antigen binding protein with a CDRH1, a CDRH2 and/or a CDRH3 of an antigen binding domain having a variable heavy chain as defined in any one of SEQ ID NOs: 2 or 3.

In any embodiment, the invention provides an antigen binding protein with a CDRL1, a CDRL2 and/or a CDRL3 of an antigen binding domain having a variable light chain as defined in any one of SEQ ID NOs: 5 or 6.

In any embodiment, the invention provides an antigen binding protein for binding to La, the protein comprising:
a CDR1, a CDR2 and a CDR3 of an antigen binding domain having a variable heavy chain as defined in SEQ ID NO: 1, and a CDR1, a CDR2 and a CDR3 of an antigen binding domain having a variable light chain as defined in SEQ ID NO: 5;
a CDR1, a CDR2 and a CDR3 of an antigen binding domain having a variable heavy chain as defined in SEQ ID NO: 1, and a CDR1, a CDR2 and a CDR3 of an antigen binding domain having a variable light chain as defined in SEQ ID NO: 6;
a CDR1, a CDR2 and a CDR3 of an antigen binding domain having a variable heavy chain as defined in SEQ ID NO: 2, and a CDR1, a CDR2 and a CDR3 of an antigen binding domain having a variable light chain as defined in SEQ ID NO: 4;
a CDR1, a CDR2 and a CDR3 of an antigen binding domain having a variable heavy chain as defined in SEQ ID NO: 2, and a CDR1, a CDR2 and a CDR3 of an antigen binding domain having a variable light chain as defined in SEQ ID NO: 5;
a CDR1, a CDR2 and a CDR3 of an antigen binding domain having a variable heavy chain as defined in SEQ ID NO: 2, and a CDR1, a CDR2 and a CDR3 of an antigen binding domain having a variable light chain as defined in SEQ ID NO: 6;
a CDR1, a CDR2 and a CDR3 of an antigen binding domain having a variable heavy chain as defined in SEQ ID NO: 3, and a CDR1, a CDR2 and a CDR3 of an antigen binding domain having a variable light chain as defined in SEQ ID NO: 4;
a CDR1, a CDR2 and a CDR3 of an antigen binding domain having a variable heavy chain as defined in SEQ ID NO: 3, and a CDR1, a CDR2 and a CDR3 of an antigen binding domain having a variable light chain as defined in SEQ ID NO: 5; or
a CDR1, a CDR2 and a CDR3 of an antigen binding domain having a variable heavy chain as defined in SEQ ID NO: 3, and a CDR1, a CDR2 and a CDR3 of an antigen binding domain having a variable light chain as defined in SEQ ID NO: 6.

In any embodiment, the antigen binding domain comprises:
i) a VH comprising a complementarity determining region CDR1 comprising or consisting of an amino acid sequence of SEQ ID NO: 7, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR2 comprising or consisting of an amino acid sequence of SEQ ID NO: 8, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR3 comprising or consisting of an amino acid sequence of SEQ ID NO: 9, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; and
ii) a VL comprising a complementarity determining region (CDR) 1 comprising or consisting of an amino acid sequence of SEQ ID NOs: 63, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR2 comprising or consisting of an amino acid sequence of SEQ ID NO: 64, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR3 comprising or consisting of an amino acid sequence of SEQ ID NO: 65, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto;
   or
iii) a VH comprising a complementarity determining region CDR1 comprising or consisting of an amino acid sequence of SEQ ID NO: 7, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR2 comprising or consisting of an amino acid sequence of SEQ ID NO: 8, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR3 comprising or consisting of an amino acid sequence of SEQ ID NO: 9, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; and
iv) a VL comprising a complementarity determining region (CDR) 1 comprising or consisting of an amino acid sequence of SEQ ID NO: 77, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR2 comprising or consisting of an amino acid sequence of SEQ ID NO: 78, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR3 comprising or consisting of an amino acid sequence of SEQ ID NO: 79, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto;
   or
v) a VH comprising a complementarity determining region CDR1 comprising or consisting of an amino acid sequence of SEQ ID NO: 21, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR2 comprising or consisting of an amino acid sequence of SEQ ID NO: 22, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR3 comprising or consisting of an amino acid sequence of SEQ ID NO: 23, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; and
vi) a VL comprising a complementarity determining region (CDR) 1 comprising or consisting of an amino acid sequence of SEQ ID NO: 49, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR2 comprising or consisting of an amino acid sequence of SEQ ID NO: 50, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR3 comprising or consisting of an amino acid sequence of SEQ ID NO: 21, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto;
   or
vii) a VH comprising a complementarity determining region CDR1 comprising or consisting of an amino acid sequence of SEQ ID NO: 21, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR2 comprising or consisting of an amino acid sequence of SEQ ID NO: 22, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR3 comprising or consisting of an amino acid sequence of SEQ ID NO: 23, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; and
viii) a VL comprising a complementarity determining region (CDR) 1 comprising or consisting of an amino acid sequence of SEQ ID NOs: 63, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR2 comprising or consisting of an amino acid sequence of SEQ ID NO: 64, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR3 comprising or consisting of an amino acid sequence of SEQ ID NO: 65, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto;
   or
ix) a VH comprising a complementarity determining region CDR1 comprising or consisting of an amino acid sequence of SEQ ID NO: 21, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR2 comprising or consisting of an amino acid sequence of SEQ ID NO: 22, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR3 comprising or consisting of an amino acid sequence of SEQ ID NO: 23, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; and
x) a VL comprising a complementarity determining region (CDR) 1 comprising or consisting of an amino acid sequence of SEQ ID NOs: 77, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR2 comprising or consisting of an amino acid sequence of SEQ ID NO: 78, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR3 comprising or consisting of an amino acid sequence of SEQ ID NO: 7965, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto;
   or
xi) a VH comprising a complementarity determining region CDR1 comprising or consisting of an amino acid sequence of SEQ ID NO: 35, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR2 comprising or consisting of an amino acid sequence of SEQ ID NO: 36, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR3 comprising or consisting of an amino acid sequence of SEQ ID NO: 37, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; and
xii) a VL comprising a complementarity determining region (CDR) 1 comprising or consisting of an amino acid sequence of SEQ ID NO: 49, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR2 comprising or consisting of an amino acid sequence of SEQ ID NO: 50, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR3 comprising or consisting of an amino acid sequence of SEQ ID NO: 21, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto;
   or
xiii) a VH comprising a complementarity determining region CDR1 comprising or consisting of an amino acid sequence of SEQ ID NO: 35, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR2 comprising or consisting of an amino acid sequence of SEQ ID NO: 36, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR3 comprising or consisting of an amino acid sequence of SEQ ID NO: 37, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; and
xiv) a VL comprising a complementarity determining region (CDR) 1 comprising or consisting of an amino acid sequence of SEQ ID NOs: 63, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR2 comprising or consisting of an amino acid sequence of SEQ ID NO: 64, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR3 comprising or consisting of an amino acid sequence of SEQ ID NO: 65, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto;
   or
xv) a VH comprising a complementarity determining region CDR1 comprising or consisting of an amino acid sequence of SEQ ID NO: 35, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR2 comprising or consisting of an amino acid sequence of SEQ ID NO: 36, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR3 comprising or consisting of an amino acid sequence of SEQ ID NO: 37, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; and
xvi) a VL comprising a complementarity determining region (CDR) 1 comprising or consisting of an amino acid sequence of SEQ ID NOs: 77, or a sequence at least about 80%, at least about 85%, at least about 90%, at least about 91%, at least about 92%, at least about 93%, at least about 94%, at least about 95%, at least about 96%, at least comprising or consisting of an amino acid sequence of SEQ ID NO: 78, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR3 comprising or consisting of an amino acid sequence of SEQ ID NO: 79, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto.

The antigen binding domain may further comprise:
when the antigen binding domain comprises i) and ii) above, a VH comprising a framework region (FR) 1 comprising an amino acid sequence of SEQ ID NO: 10, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 11, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 12, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 13, or a sequence at least about 80%, at least 85%, at least 90%, at least 91%, at least 92%, at least 93%, at least 94%, at least 95%, at least 96%, at least 97%, at least 98% or at least 99% thereto; and
a VL comprising a FR1 comprising an amino acid sequence of SEQ ID NO: 66, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 67, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 68, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 69, or a sequence at least about 80%, at least 85%, at least 90%, at least 91%, at least 92%, at least 93%, at least 94%, at least 95%, at least 96%, at least 97%, at least 98% or at least 99% thereto;
   or
when the antigen binding domain comprises iii) and iv) above, a VH comprising a framework region (FR) 1 comprising an amino acid sequence of SEQ ID NO: 10, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 11, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 12, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 13, or a sequence at least about 80%, at least 85%, at least 90%, at least 91%, at least 92%, at least 93%, at least 94%, at least 95%, at least 96%, at least 97%, at least 98% or at least 99% thereto; and
a VL comprising a FR1 comprising an amino acid sequence of SEQ ID NO: 80, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 81, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 82, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 83, or a sequence at least about 80%, at least 85%, at least 90%, at least 91%, at least 92%, at least 93%, at least 94%, at least 95%, at least 96%, at least 97%, at least 98% or at least 99% thereto;
   or
when the antigen binding domain comprises v) and vi) above, a VH comprising a framework region (FR) 1 comprising an amino acid sequence of SEQ ID NO: 24, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 25, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 26, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 27, or a sequence at least about 80%, at least 85%, at least 90%, at least 91%, at least 92%, at least 93%, at least 94%, at least 95%, at least 96%, at least 97%, at least 98% or at least 99% thereto; and
a VL comprising a FR1 comprising an amino acid sequence of SEQ ID NO: 52, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 53, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 54, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 55, or a sequence at least about 80%, at least 85%, at least 90%, at least 91%, at least 92%, at least 93%, at least 94%, at least 95%, at least 96%, at least 97%, at least 98% or at least 99% thereto;
   or
when the antigen binding domain comprises vii) and viii) above, a VH comprising a framework region (FR) 1 comprising an amino acid sequence of SEQ ID NO: 24, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 25, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 26, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 27, or a sequence at least about 80%, at least 85%, at least 90%, at least 91%, at least 92%, at least 93%, at least 94%, at least 95%, at least 96%, at least 97%, at least 98% or at least 99% thereto; and
a VL comprising a FR1 comprising an amino acid sequence of SEQ ID NO: 66, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 67, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 68, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 69, or a sequence at least about 80%, at least 85%, at least 90%, at least 91%, at least 92%, at least 93%, at least 94%, at least 95%, at least 96%, at least 97%, at least 98% or at least 99% thereto;
   or
when the antigen binding domain comprises ix) and x) above, a VH comprising a framework region (FR) 1 comprising an amino acid sequence of SEQ ID NO: 24, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 25, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 26, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 27, or a sequence at least about 80%, at least 85%, at least 90%, at least 91%, at least 92%, at least 93%, at least 94%, at least 95%, at least 96%, at least 97%, at least 98% or at least 99% thereto; and
a VL comprising a FR1 comprising an amino acid sequence of SEQ ID NO: 80, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 81, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 82, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 83, or a sequence at least about 80%, at least 85%, at least 90%, at least 91%, at least 92%, at least 93%, at least 94%, at least 95%, at least 96%, at least 97%, at least 98% or at least 99% thereto;
   or
when the antigen binding domain comprises xi) and xii) above, a VH comprising a framework region (FR) 1 comprising an amino acid sequence of SEQ ID NO: 38, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 39, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 40, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 41, or a sequence at least about 80%, at least 85%, at least 90%, at least 91%, at least 92%, at least 93%, at least 94%, at least 95%, at least 96%, at least 97%, at least 98% or at least 99% thereto; and
a VL comprising a FR1 comprising an amino acid sequence of SEQ ID NO: 52, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 53, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 54, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 55, or a sequence at least about 80%, at least 85%, at least 90%, at least 91%, at least 92%, at least 93%, at least 94%, at least 95%, at least 96%, at least 97%, at least 98% or at least 99% thereto;
   or
when the antigen binding domain comprises xiii) and xiv) above, a VH comprising a framework region (FR) 1 comprising an amino acid sequence of SEQ ID NO: 38, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 39, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 40, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 41, or a sequence at least about 80%, at least 85%, at least 90%, at least 91%, at least 92%, at least 93%, at least 94%, at least 95%, at least 96%, at least 97%, at least 98% or at least 99% thereto; and
a VL comprising a FR1 comprising an amino acid sequence of SEQ ID NO: 66, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 67, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 68, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 69, or a sequence at least about 80%, at least 85%, at least 90%, at least 91%, at least 92%, at least 93%, at least 94%, at least 95%, at least 96%, at least 97%, at least 98% or at least 99% thereto;
   or
when the antigen binding domain comprises xv) and xvi) above, a VH comprising a framework region (FR) 1 comprising an amino acid sequence of SEQ ID NO: 38, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 39, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 40, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 41, or a sequence at least about 80%, at least 85%, at least 90%, at least 91%, at least 92%, at least 93%, at least 94%, at least 95%, at least 96%, at least 97%, at least 98% or at least 99% thereto; and
a VL comprising a FR1 comprising an amino acid sequence of SEQ ID NO: 80, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 81, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 82, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 83, or a sequence at least about 80%, at least 85%, at least 90%, at least 91%, at least 92%, at least 93%, at least 94%, at least 95%, at least 96%, at least 97%, at least 98% or at least 99% thereto.

In an embodiment, the antigen binding domain comprises:
a) a VH comprising a complementarity determining region CDR1 comprising or consisting of an amino acid sequence of SEQ ID NO: 14, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR2 comprising or consisting of an amino acid sequence of SEQ ID NO: 15, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR3 comprising or consisting of an amino acid sequence of SEQ ID NO: 16, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; and
b) a VL comprising a complementarity determining region (CDR) 1 comprising or consisting of an amino acid sequence of SEQ ID NO: 70, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR2 comprising or consisting of an amino acid sequence of SEQ ID NO: 71, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR3 comprising or consisting of an amino acid sequence of SEQ ID NO: 72, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto;
   or
c) a VH comprising a complementarity determining region CDR1 comprising or consisting of an amino acid sequence of SEQ ID NO: 14, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR2 comprising or consisting of an amino acid sequence of SEQ ID NO: 15, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR3 comprising or consisting of an amino acid sequence of SEQ ID NO: 16, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; and
d) a VL comprising a complementarity determining region (CDR) 1 comprising or consisting of an amino acid sequence of SEQ ID NO: 84, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR2 comprising or consisting of an amino acid sequence of SEQ ID NO: 85, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR3 comprising or consisting of an amino acid sequence of SEQ ID NO: 86, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto;
   or
e) a VH comprising a complementarity determining region CDR1 comprising or consisting of an amino acid sequence of SEQ ID NO: 28, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR2 comprising or consisting of an amino acid sequence of SEQ ID NO: 29, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR3 comprising or consisting of an amino acid sequence of SEQ ID NO: 30, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; and
f) a VL comprising a complementarity determining region (CDR) 1 comprising or consisting of an amino acid sequence of SEQ ID NO: 56, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR2 comprising or consisting of an amino acid sequence of SEQ ID NO: 57, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR3 comprising or consisting of an amino acid sequence of SEQ ID NO: 58, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto;
   or
g) a VH comprising a complementarity determining region CDR1 comprising or consisting of an amino acid sequence of SEQ ID NO: 28, or a sequence at least about 80%, at least about 85%, at least about 90%, at least about 91%, at least about 92%, at least about 93%, at least about 94%, at least about 95%, at least about 96%, at least comprising or consisting of an amino acid sequence of SEQ ID NO: 29, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR3 comprising or consisting of an amino acid sequence of SEQ ID NO: 30, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; and
h) a VL comprising a complementarity determining region (CDR) 1 comprising or consisting of an amino acid sequence of SEQ ID NO: 70, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR2 comprising or consisting of an amino acid sequence of SEQ ID NO: 71, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR3 comprising or consisting of an amino acid sequence of SEQ ID NO: 72, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto;
   or
i) a VH comprising a complementarity determining region CDR1 comprising or consisting of an amino acid sequence of SEQ ID NO: 28, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR2 comprising or consisting of an amino acid sequence of SEQ ID NO: 29, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR3 comprising or consisting of an amino acid sequence of SEQ ID NO: 30, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; and
j) a VL comprising a complementarity determining region (CDR) 1 comprising or consisting of an amino acid sequence of SEQ ID NO: 84, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR2 comprising or consisting of an amino acid sequence of SEQ ID NO: 85, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR3 comprising or consisting of an amino acid sequence of SEQ ID NO: 86, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto;
   or
k) a VH comprising a complementarity determining region CDR1 comprising or consisting of an amino acid sequence of SEQ ID NO: 42, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR2 comprising or consisting of an amino acid sequence of SEQ ID NO: 43, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR3 comprising or consisting of an amino acid sequence of SEQ ID NO: 44, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; and
l) a VL comprising a complementarity determining region (CDR) 1 comprising or consisting of an amino acid sequence of SEQ ID NO: 56, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR2 comprising or consisting of an amino acid sequence of SEQ ID NO: 57, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR3 comprising or consisting of an amino acid sequence of SEQ ID NO: 58, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto;
   or
m) a VH comprising a complementarity determining region CDR1 comprising or consisting of an amino acid sequence of SEQ ID NO: 42, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR2 comprising or consisting of an amino acid sequence of SEQ ID NO: 43, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR3 comprising or consisting of an amino acid sequence of SEQ ID NO: 44, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; and
n) a VL comprising a complementarity determining region (CDR) 1 comprising or consisting of an amino acid sequence of SEQ ID NO: 70, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR2 comprising or consisting of an amino acid sequence of SEQ ID NO: 71, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR3 comprising or consisting of an amino acid sequence of SEQ ID NO: 72, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto;
   or
o) a VH comprising a complementarity determining region CDR1 comprising or consisting of an amino acid sequence of SEQ ID NO: 42, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR2 comprising or consisting of an amino acid sequence of SEQ ID NO: 43, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR3 comprising or consisting of an amino acid sequence of SEQ ID NO: 44, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; and
p) a VL comprising a complementarity determining region (CDR) 1 comprising or consisting of an amino acid sequence of SEQ ID NOs: 84, or a sequence at least about 80%, at least about 85%, at least about 90%, at least about 91%, at least about 92%, at least about 93%, at least about 94%, at least about 95%, at least about 96%, at least comprising or consisting of an amino acid sequence of SEQ ID NO: 85, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; a CDR3 comprising or consisting of an amino acid sequence of SEQ ID NO: 86, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto.

The antigen binding domain may further comprise:
when the antigen binding domain comprises a) and b) above, a VH comprising a framework region (FR) 1 comprising an amino acid sequence of SEQ ID NO: 10, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 11, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 12, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 13, or a sequence at least about 80%, at least 85%, at least 90%, at least 91%, at least 92%, at least 93%, at least 94%, at least 95%, at least 96%, at least 97%, at least 98% or at least 99% thereto; and
a VL comprising a FR1 comprising an amino acid sequence of SEQ ID NO: 66, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 67, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 68, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 69, or a sequence at least about 80%, at least 85%, at least 90%, at least 91%, at least 92%, at least 93%, at least 94%, at least 95%, at least 96%, at least 97%, at least 98% or at least 99% thereto;
   or
when the antigen binding domain comprises c) and d above, a VH comprising a framework region (FR) 1 comprising an amino acid sequence of SEQ ID NO: 10, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 11, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 12, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 13, or a sequence at least about 80%, at least 85%, at least 90%, at least 91%, at least 92%, at least 93%, at least 94%, at least 95%, at least 96%, at least 97%, at least 98% or at least 99% thereto; and
a VL comprising a FR1 comprising an amino acid sequence of SEQ ID NO: 80, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 81, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 82, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 83, or a sequence at least about 80%, at least 85%, at least 90%, at least 91%, at least 92%, at least 93%, at least 94%, at least 95%, at least 96%, at least 97%, at least 98% or at least 99% thereto;
   or
when the antigen binding domain comprises e) and f) above, a VH comprising a framework region (FR) 1 comprising an amino acid sequence of SEQ ID NO: 24, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 25, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 26, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 27, or a sequence at least about 80%, at least 85%, at least 90%, at least 91%, at least 92%, at least 93%, at least 94%, at least 95%, at least 96%, at least 97%, at least 98% or at least 99% thereto; and
a VL comprising a FR1 comprising an amino acid sequence of SEQ ID NO: 52, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 53, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 54, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 55, or a sequence at least about 80%, at least 85%, at least 90%, at least 91%, at least 92%, at least 93%, at least 94%, at least 95%, at least 96%, at least 97%, at least 98% or at least 99% thereto;
   or
when the antigen binding domain comprises g) and h) above, a VH comprising a framework region (FR) 1 comprising an amino acid sequence of SEQ ID NO: 24, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 25, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 26, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 27, or a sequence at least about 80%, at least 85%, at least 90%, at least 91%, at least 92%, at least 93%, at least 94%, at least 95%, at least 96%, at least 97%, at least 98% or at least 99% thereto; and
a VL comprising a FR1 comprising an amino acid sequence of SEQ ID NO: 66, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 67, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 68, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 69, or a sequence at least about 80%, at least 85%, at least 90%, at least 91%, at least 92%, at least 93%, at least 94%, at least 95%, at least 96%, at least 97%, at least 98% or at least 99% thereto;
   or
when the antigen binding domain comprises i) and j) above, a VH comprising a framework region (FR) 1 comprising an amino acid sequence of SEQ ID NO: 24, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 25, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 26, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 27, or a sequence at least about 80%, at least 85%, at least 90%, at least 91%, at least 92%, at least 93%, at least 94%, at least 95%, at least 96%, at least 97%, at least 98% or at least 99% thereto; and
a VL comprising a FR1 comprising an amino acid sequence of SEQ ID NO: 80, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 81, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 82, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 83, or a sequence at least about 80%, at least 85%, at least 90%, at least 91%, at least 92%, at least 93%, at least 94%, at least 95%, at least 96%, at least 97%, at least 98% or at least 99% thereto;
   or
when the antigen binding domain comprises k) and l) above, a VH comprising a framework region (FR) 1 comprising an amino acid sequence of SEQ ID NO: 38, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 39, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 40, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 41, or a sequence at least about 80%, at least 85%, at least 90%, at least 91%, at least 92%, at least 93%, at least 94%, at least 95%, at least 96%, at least 97%, at least 98% or at least 99% thereto; and
a VL comprising a FR1 comprising an amino acid sequence of SEQ ID NO: 52, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 53, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 54, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 55, or a sequence at least about 80%, at least 85%, at least 90%, at least 91%, at least 92%, at least 93%, at least 94%, at least 95%, at least 96%, at least 97%, at least 98% or at least 99% thereto;
   or
when the antigen binding domain comprises m) and n) above, a VH comprising a framework region (FR) 1 comprising an amino acid sequence of SEQ ID NO: 38, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 39, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 40, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 41, or a sequence at least about 80%, at least 85%, at least 90%, at least 91%, at least 92%, at least 93%, at least 94%, at least 95%, at least 96%, at least 97%, at least 98% or at least 99% thereto; and
a VL comprising a FR1 comprising an amino acid sequence of SEQ ID NO: 66, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 67, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 68, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 69, or a sequence at least about 80%, at least 85%, at least 90%, at least 91%, at least 92%, at least 93%, at least 94%, at least 95%, at least 96%, at least 97%, at least 98% or at least 99% thereto;
   or
when the antigen binding domain comprises o) and p) above, a VH comprising a framework region (FR) 1 comprising an amino acid sequence of SEQ ID NO: 38, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 39, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 40, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 41, or a sequence at least about 80%, at least 85%, at least 90%, at least 91%, at least 92%, at least 93%, at least 94%, at least 95%, at least 96%, at least 97%, at least 98% or at least 99% thereto; and
a VL comprising a FR1 comprising an amino acid sequence of SEQ ID NO: 80, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 81, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 82, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 83, or a sequence at least about 80%, at least 85%, at least 90%, at least 91%, at least 92%, at least 93%, at least 94%, at least 95%, at least 96%, at least 97%, at least 98% or at least 99% thereto.

In any embodiment, the antigen binding protein comprises a variable heavy chain comprising the amino acid sequence as set forth in SEQ ID NO: 1, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; and a variable light chain comprising the amino acid sequence as set forth in SEQ ID NO: 5; or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; wherein the variable heavy and/or light chains comprise no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19 or no more than 20 amino acid residue substitutions, deletions, or additions or combination thereof, wherein the substitutions, deletions, or additions or combination thereof are not in the CDRs, and wherein the antigen binding protein retains the ability to bind to La.

In any embodiment, the antigen binding protein comprises a variable heavy chain comprising the amino acid sequence as set forth in SEQ ID NO: 1, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; and a variable light chain comprising the amino acid sequence as set forth in SEQ ID NO: 6; or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; wherein the variable heavy and/or light chains comprise no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19 or no more than 20 amino acid residue substitutions, deletions, or additions or combination thereof, wherein the substitutions, deletions, or additions or combination thereof are not in the CDRs, and wherein the antigen binding protein retains the ability to bind to La.

In any embodiment, the antigen binding protein comprises a variable heavy chain comprising the amino acid sequence as set forth in SEQ ID NO: 2 or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; and a variable light chain comprising the amino acid sequence as set forth in SEQ ID NO: 4; or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; wherein the variable heavy and/or light chains comprise no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19 or no more than 20 amino acid residue substitutions, deletions, or additions or combination thereof, wherein the substitutions, deletions, or additions or combination thereof are not in the CDRs, and wherein the antigen binding protein retains the ability to bind to La.

In any embodiment, the antigen binding protein comprises a variable heavy chain comprising the amino acid sequence as set forth in SEQ ID NO: 2, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; and a variable light chain comprising the amino acid sequence as set forth in SEQ ID NO: 5; or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; wherein the variable heavy and/or light chains comprise no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19 or no more than 20 amino acid residue substitutions, deletions, or additions or combination thereof, wherein the substitutions, deletions, or additions or combination thereof are not in the CDRs, and wherein the antigen binding protein retains the ability to bind to La.

In any embodiment, the antigen binding protein comprises a variable heavy chain comprising the amino acid sequence as set forth in SEQ ID NO: 2, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; and a variable light chain comprising the amino acid sequence as set forth in SEQ ID NO: 6 or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; wherein the variable heavy and/or light chains comprise no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19 or no more than 20 amino acid residue substitutions, deletions, or additions or combination thereof, wherein the substitutions, deletions, or additions or combination thereof are not in the CDRs, and wherein the antigen binding protein retains the ability to bind to La.

In any embodiment, the antigen binding protein comprises a variable heavy chain comprising the amino acid sequence as set forth in any one of SEQ ID NO: 3, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; and a variable light chain comprising the amino acid sequence as set forth in SEQ ID NO: 4; or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; wherein the variable heavy and/or light chains comprise no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19 or no more than 20 amino acid residue substitutions, deletions, or additions or combination thereof, wherein the substitutions, deletions, or additions or combination thereof are not in the CDRs, and wherein the antigen binding protein retains the ability to bind to La.

In any embodiment, the antigen binding protein comprises a variable heavy chain comprising the amino acid sequence as set forth in SEQ ID NO: 3, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; and a variable light chain comprising the amino acid sequence as set forth in SEQ ID NO: 5; or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; wherein the variable heavy and/or light chains comprise no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19 or no more than 20 amino acid residue substitutions, deletions, or additions or combination thereof, wherein the substitutions, deletions, or additions or combination thereof are not in the CDRs, and wherein the antigen binding protein retains the ability to bind to La.

In any embodiment, the antigen binding protein comprises a variable heavy chain comprising the amino acid sequence as set forth in SEQ ID NO: 3, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; and a variable light chain comprising the amino acid sequence as set forth in SEQ ID NO: 6; or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; wherein the variable heavy and/or light chains comprise no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19 or no more than 20 amino acid residue substitutions, deletions, or additions or combination thereof, wherein the substitutions, deletions, or additions or combination thereof are not in the CDRs, and wherein the antigen binding protein retains the ability to bind to La.

In a further embodiment, the invention provides an antigen binding protein for binding to La/SSB, wherein the protein comprises a VH comprising a framework region (FR) 1 comprising an amino acid sequence of SEQ ID NO: 24, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 25, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 26, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 27, or a sequence at least about 80%, at least 85%, at least 90%, at least 91%, at least 92%, at least 93%, at least 94%, at least 95%, at least 96%, at least 97%, at least 98% or at least 99% thereto; and
a VL comprising a FR1 comprising an amino acid sequence of SEQ ID NO: 66, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 67, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 68, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 69, or a sequence at least about 80%, at least 85%, at least 90%, at least 91%, at least 92%, at least 93%, at least 94%, at least 95%, at least 96%, at least 97%, at least 98% or at least 99% thereto.

In a further embodiment, the invention provides an antigen binding protein for binding to La/SSB, wherein the protein comprises a VH comprising a complementarity determining region CDR1 comprising or consisting of an amino acid sequence of SEQ ID NO: 35; a CDR2 comprising or consisting of an amino acid sequence of SEQ ID NO: 36; a CDR3 comprising or consisting of an amino acid sequence of SEQ ID NO: 37; and
a VL comprising a complementarity determining region (CDR) 1 comprising or consisting of an amino acid sequence of SEQ ID NO: 77; a CDR2 comprising or consisting of an amino acid sequence of SEQ ID NO: 78; a CDR3 comprising or consisting of an amino acid sequence of SEQ ID NO: 79,
wherein the protein further comprises:
a VH comprising a framework region (FR) 1 comprising an amino acid sequence of SEQ ID NO: 38, or a sequence at least about 87%, at least 88% at least 89%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 39, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 40, or a sequence at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 41; and
a VL comprising a FR1 comprising an amino acid sequence of SEQ ID NO: 80, or a sequence at least 70%, at least 71%, at least 72%, at least 73%, at least 74%, at least 75%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 81, or a sequence at least 94%, at least 95%, at least 96%, at least 97%, at least 98%, or at least 99% identical thereto; a FR3 comprising an amino acid sequence of SEQ ID NO: 82, or a sequence at least 94%, at least 95%, at least 96%, at least 97%, at least 98% or at least 99% identical thereto, and a FR4 comprising an amino acid sequence of SEQ ID NO: 83, or a sequence at least about 80%, at least 85%, at least 90%, at least 91%, at least 92%, at least 93%, at least 94%, at least 95%, at least 96%, at least 97%, at least 98% or at least 99% thereto.

In any embodiment, the invention provides an antigen binding protein for binding to La, the protein comprising:
an antigen binding domain having a variable heavy chain as defined in SEQ ID NO: 1, and an antigen binding domain having a variable light chain as defined in SEQ ID NO: 5;
an antigen binding domain having a variable heavy chain as defined in SEQ ID NO: 1, and an antigen binding domain having a variable light chain as defined in SEQ ID NO: 6;
an antigen binding domain having a variable heavy chain as defined in SEQ ID NO: 2, and an antigen binding domain having a variable light chain as defined in SEQ ID NO: 4;
an antigen binding domain having a variable heavy chain as defined in SEQ ID NO: 2, and an antigen binding domain having a variable light chain as defined in SEQ ID NO: 5;
an antigen binding domain having a variable heavy chain as defined in SEQ ID NO: 2, and an antigen binding domain having a variable light chain as defined in SEQ ID NO: 6;
an antigen binding domain having a variable heavy chain as defined in SEQ ID NO: 3, and an antigen binding domain having a variable light chain as defined in SEQ ID NO: 4;
an antigen binding domain having a variable heavy chain as defined in SEQ ID NO: 3, and an antigen binding domain having a variable light chain as defined in SEQ ID NO: 5; or
an antigen binding domain having a variable heavy chain as defined in SEQ ID NO: 3, and an antigen binding domain having a variable light chain as defined in SEQ ID NO: 6.

As described herein, the antigen binding protein may be in the form of:
(i) a single domain antibody (sdAb);
(ii) a single chain Fv fragment (scFv);
(iii) a dimeric scFv (di-scFv); or
(iv) one of (ii) or (iii) linked to a constant region of an antibody, Fc or a heavy chain constant domain (CH) 2 and/or CH3.

Further, as described herein, the antigen binding protein may be in the form of:
(i) a diabody;
(ii) a triabody;
(iii) a tetrabody;
(iv) a Fab;
(v) a F(ab′)2;
(vi) a Fv;
(vii) a bispecific antibody or other form of multispecific antibody; or
(viii) one of (i) to (vii) linked to a constant region of an antibody, Fc or a heavy chain constant domain (CH) 2 and/or CH3.

The foregoing antigen binding proteins can also be referred to as antigen binding domains of antibodies.

Preferably, an antigen binding protein as described herein is an antibody or antigen binding fragment thereof. Typically, the antigen binding protein is an antibody, for example, a monoclonal antibody. The antigen binding protein may be in the form of a recombinant or modified antibody (e.g., chimeric antibody, humanised antibody, human antibody, CDR-grafted antibody, primatised antibody, de-immunised antibody, synhumanised antibody, half-antibody, bispecific antibody, trispecific antibody or multispecific antibody). The antibody may further comprise a chemical modification, such as conjugation to an active agent or radiolabel, or an agent for improving solubility or other modification described herein.

As used herein the antigen binding protein may be a variable domain.

The invention provides an antigen binding protein including, consisting essentially of or consisting of an amino acid sequence of (in order of N to C terminus or C to N terminus):
SEQ ID NO: 1 and 5;
SEQ ID NO: 1 and 6;
SEQ ID NO: 2 and 4;
SEQ ID NO: 2 and 5;
SEQ ID NO: 2 and 6;
SEQ ID NO: 3 and 4;
SEQ ID NO: 3 and 5; or
SEQ ID NO: 3 and 6.

As used herein, the complementarity determining region sequences (CDRs) of an antigen binding protein of the invention may be defined according to the IMGT, Chothia or Kabat numbering systems, or any other CDR numbering system known to the skilled person.

The invention provides an antigen binding protein as described herein wherein an amino acid sequence forming one or more of FR1, CDR1, FR2, CDR2, FR3, CDR3 and FR4 is a human sequence.

The invention provides an anti-La binding protein, for example an immunoglobulin variable domain, antibody, dab, scFv, Fab, Fab′, F(ab′)2, Fv fragment, diabody, triabody, linear antibody, single-chain antibody molecule, or multispecific antibody comprising an antigen binding protein having a sequence as described herein, or including a CDR and/or FR sequence as described herein.

An antigen binding protein as described herein may comprise a human constant region, e.g., an IgG constant region, such as an IgG₁, IgG₂, IgG₃ or IgG₄ constant region or mixtures thereof. In the case of an antibody or protein comprising a VH and a VL, the VH can be linked to a heavy chain constant region and the VL can be linked to a light chain constant region.

In one example, an antigen binding protein as described herein comprises a constant region of an IgG₄ antibody or a stabilised constant region of an IgG₄ antibody. In one example, the protein or antibody comprises an IgG₄ constant region with a proline at position 241 (according to the numbering system of Kabat (Kabat et al., Sequences of Proteins of Immunological Interest Washington DC United States Department of Health and Human Services, 1987 and/or 1991)).

In one example, an antigen binding protein as described herein or a composition of an antigen binding protein as described herein, comprises a heavy chain constant region, comprising a stabilised heavy chain constant region, comprising a mixture of sequences fully or partially with or without the C-terminal lysine residue.

In one example, an antigen binding protein comprises a V_{H} disclosed herein linked or fused to an IgG₄ constant region or stabilised IgG₄ constant region (e.g., as discussed above) and the V_{L} is linked to or fused to a kappa light chain constant region.

In any aspect of the present invention, the antibody is a naked antibody. Specifically, the antibody is in a non-conjugated form and is not adapted to form a conjugate.

The invention provides a fusion protein comprising an antigen binding protein, for example an immunoglobulin variable domain, antibody, dab, scFv, Fab, Fab′, F(ab′)2, Fv fragment, diabody, triabody, linear antibody, single-chain antibody molecule, or multispecific antibody as described herein.

The invention also provides a conjugate in the form of an antigen binding protein, immunoglobulin variable domain, antibody, dab, scFv, Fab, Fab′, F(ab′)2, Fv fragment, diabody, triabody, linear antibody, single-chain antibody molecule, or multispecific antibody or fusion protein as described herein, conjugated to a label or a cytotoxic agent. The cytotoxic agent may be a chemotherapeutic agent or other agent used for the treatment of a disease, such as cancer. In some aspects, the antigen binding protein is not directly or indirectly conjugated to a cytotoxic agent, but the antigen binding protein is associated with a cytotoxic agent and brings about cell killing by directing the cytotoxic agent to a cell expressing La/SSB.

In another embodiment, the antigen binding protein comprises an Fc region that is engineered to:
increase the in vitro or in vivo half-life;
have an increased capacity to induce antibody-dependent cell mediated cytotoxicity (ADCC), antibody-dependent cellular phagocytosis (ADCP) or complement-dependent cytotoxicity;
reduce effector function; or
increase co-engagement of the antigen binding protein.

Mutations, deletions or modifications of amino acids in the Fc region which affect half-life, ADCC, ADCP or complement-dependent cytotoxicity, effector function are known to the skilled person and further described herein.

The functional characteristics of an antigen binding protein of the invention will be taken to apply mutatis mutandis to an antibody of the invention.

The present invention also provides an expression construct encoding an antigen binding protein, immunoglobulin variable domain, antibody, dab, scFv, Fab, Fab′, F(ab′)2, Fv fragment, diabody, triabody, linear antibody, single-chain antibody molecule, or multispecific antibody or fusion protein as described herein.

In aspects of the invention directed to multiple polypeptide chains that form an antigen binding protein, an expression construct comprises a nucleic acid encoding a polypeptide comprising, e.g., a VH operably linked to a promoter and a nucleic acid encoding a polypeptide comprising, e.g., a VL operably linked to a promoter.

In another example, the expression construct is a bicistronic expression construct, e.g., comprising the following operably linked components in 5′ to 3′ order:
(i) a promoter
(ii) a nucleic acid encoding a first polypeptide;
(iii) an internal ribosome entry site; and
(iv) a nucleic acid encoding a second polypeptide,
wherein the first polypeptide comprises a VH and the second polypeptide comprises a VL, or vice versa.

The present invention also contemplates separate expression constructs one of which encodes a first polypeptide comprising a VH and another of which encodes a second polypeptide comprising a VL. For example, the present invention also provides a composition comprising:
(i) a first expression construct comprising a nucleic acid encoding a polypeptide comprising a VH operably linked to a promoter; and
(ii) a second expression construct comprising a nucleic acid encoding a polypeptide comprising a VL operably linked to a promoter.

The invention provides a cell comprising a vector or nucleic acid described herein. Preferably, the cell is isolated, substantially purified or recombinant. In one example, the cell comprises the expression construct of the invention or:
(i) a first expression construct comprising a nucleic acid encoding a polypeptide comprising a VH operably linked to a promoter; and
(ii) a second expression construct comprising a nucleic acid encoding a polypeptide comprising a VL operably linked to a promoter,
wherein the first and second polypeptides associate to form an antigen binding protein of the present invention.

Examples of cells of the present invention include bacterial cells, yeast cells, insect cells or mammalian cells.

The invention provides a nucleic acid encoding an antigen binding protein, for example an immunoglobulin variable domain, antibody, dab, scFv, Fab, Fab′, F(ab′)2, Fv fragment, diabody, triabody, linear antibody, single-chain antibody molecule, or multispecific antibody, fusion protein or conjugate as described herein.

The invention provides a vector comprising a nucleic acid described herein. Preferably, the nucleic acid has a nucleotide sequence that encodes any one or more of the amino acid sequences corresponding to SEQ ID NO: 1 to 6.

The invention provides a cell comprising a vector or nucleic acid described herein.

In another embodiment there is provided an animal or tissue derived therefrom comprising a cell described herein.

The invention provides a pharmaceutical composition comprising an immunoglobulin variable domain, antibody, dab, scFv, Fab, Fab′, F(ab′)2, Fv fragment, diabody, triabody, linear antibody, single-chain antibody molecule, or multispecific antibody, fusion protein, conjugate, nucleic acid, vector or a cell as described herein and a pharmaceutically acceptable carrier, diluent or excipient.

The invention provides a diagnostic composition comprising an antigen binding protein, immunoglobulin variable domain, antibody, dab, scFv, Fab, Fab′, F(ab′)2, Fv fragment, diabody, triabody, linear antibody, single-chain antibody molecule, or multispecific antibody, fusion protein or conjugate, as described herein, a diluent and optionally a label. The label may be any suitable label for enabling detection of the antigen binding protein, immunoglobulin variable domain, antibody, dab, scFv, Fab, Fab′, F(ab′)2, Fv fragment, diabody, triabody, linear antibody, single-chain antibody molecule, or multispecific antibody, fusion protein or conjugate (such as a fluorescent dye or radiolabel).

The invention provides a kit or article of manufacture comprising an immunoglobulin variable domain, antibody, dab, scFv, Fab, Fab′, F(ab′)2, Fv fragment, diabody, triabody, linear antibody, single-chain antibody molecule, or multispecific antibody, fusion protein or conjugate as described herein.

The invention provides use of a sequence according to one or more or all of CDR1, CDR2, CDR3, FR1, FR2, FR3 and FR4 as described herein to produce an antigen binding protein for binding to La.

The invention provides a method for producing an antigen binding protein for binding to La as described herein comprising expressing a nucleic acid as described herein in a cell or animal as described herein.

An antigen binding protein as described herein may be purified, substantially purified, isolated and/or recombinant.

The invention also provides an immunoglobulin for binding to La, wherein the immunoglobulin comprises an antigen binding protein as herein described.

Preferably, the immunoglobulin is conjugated to a radionuclide. The radionuclide may be for enabling detection of the immunoglobulin or may be for delivery of a therapeutically effective dose of radiation to a target cell that is capable of being bound by the immunoglobulin.

As used herein the terms “radionuclide”, “radiolabel” and “radioisotope” may be used interchangeably.

Examples of radionuclides include: actinium-225 (²²⁵Ac), astatine-211 (²¹¹At), bismuth-212 and bismuth-213 (²¹²Bi, ²¹³Bi), copper-64 and copper-67 (⁶⁴Cu, ⁶⁷Cu), fluorine-18 (¹⁸F), gallium-67 and gallium-68 (⁶⁷Ga and ⁶⁸Ga), indium-111 (¹¹¹In), iodine-123, -124, -125 or -131 (¹²³I, ¹²⁴I, ¹²⁵I, ¹³¹I), lead-212 (²¹²Pb), lutetium-177 (¹⁷⁷Lu), radium-223 (²²³Ra), rhenium-188 (¹⁸⁸Re), samarium-153 (¹⁵³Sm), scandium-44 and scandium-47 (⁴⁴Sc, ⁴⁷Sc), strontium-90 (⁹⁰Sr), technetium-99 (^{99m}Tc), terbium-149, terbium-152, terbium-155 and terbium-159, yttrium-86 and yttrium-90 (⁸⁶Y, ⁹⁰Y), zirconium-89 (⁸⁹Zr).

The immunoglobulin may be directly conjugated to the radionuclide, or may be conjugated via a linker. The radionuclide may be conjugated to the immunoglobulin directly or indirectly, e.g. by halogenation of amino acid residues. Preferably, the radionuclide is indirectly conjugated to the immunoglobulin by way of a linker or chelator moiety. In one example, the immunuglobulin is conjugated to a chelating moiety, selected from the group consisting of: TMT (6,6″-bis[N,N″,N′-tetra(carboxymethyl)aminomethyl)-4′-(3-amino-4-methoxyphenyl)-2,2′:6′,2″-terpyridine), DOTA (1,4,7,10-tetraazacyclododecane-NN′,N″(N′″-tetraacetic acid), TCMC, DO3A, CB-DO2A, NOTA, Diamsar, DTPA, CHX-A″-DTPA, TETE, Te2A, HBED, DFO, DFOsq, DFO-NCS and HOPO, a chelator as described in WO 2022/133537 (incorporated herein by reference) or other chelating agent as described herein.

In another example, the immunoglobulin is conjugated to a bifunctional linker, for example, bromoacetyl, thiols, succinimide ester, TFP ester, a maleimide, or using any amine or thiol-modifying chemistry known in the art.

In addition, or in the alternative, the immunoglobulin may also comprise one or more amino acid substitutions in the Fc region of the immunoglobulin for reducing affinity of the immunoglobulin for the neonatal Fc receptor (FcRN), thereby reducing the serum half-life of the immunoglobulin compared to an immunoglobulin that does not comprise the amino acid substitutions (or compared to a wild-type immunoglobulin).

The one or more amino acid substitutions may be a substitution in the heavy chain constant region 2 (CH2) of immunoglobulin, or may be a substitution in the heavy chain constant region 3 (CH3) of the immunoglobulin, or a combination thereof.

In certain preferred embodiments, the one or more amino acid substitutions may be at one or more of residues His310, His433, His435, His436, or Ile253 of the Fc region of an immunoglobulin (numbering per Kabat nomenclature). Preferably, the amino acid substitutions comprise a substitution in the heavy chain constant region at positions His310 or at His435. More preferably, the amino acid substitutions are at both His310 and His435.

In certain embodiments, the immunoglobulin retains the ability to bind to one or more Fc-gamma receptors and accordingly, in certain embodiments the modified antibody retains the ability to stimulate effector responses (including ADCC).

In alternative embodiments, the one or more amino acid modifications which reduce the affinity for the FcRn receptor also reduce the affinity for the Fc gamma receptors. The immunoglobulin may further comprise one or more amino acid substitutions compared a wild-type antibody of the class IgG, wherein the amino acid substitutions further reduce the affinity of the antibody for one or more Fc gamma receptors.

In a further embodiment, the immunoglobulin may further comprise one or more amino acid substitutions compared a wild-type antibody of the class IgG, wherein the amino acid substitutions increase the stability of the CH1-CH2 hinge region in the modified antibody compared to a wild-type antibody of the class IgG.

The antigen binding proteins of the present invention also find utility in various applications, both diagnostic and therapeutic.

In a further aspect, there is therefore provided a method of determining the efficacy of a cytotoxic treatment, the method comprising:
administering an antigen binding protein as described herein to a subject who has received a treatment with a cytotoxic agent, wherein the antigen binding protein comprises a detectable moiety for enabling detection thereof,
detecting the antigen binding protein in the subject
determining that the treatment has been effective when the detection of the antigen binding protein in the subject is above a background level;
determining that the treatment has not been effective when the detection of the antigen binding protein in the subject is at the same or at a lower level compared to a background level
   thereby determining the efficacy of the cytotoxic treatment.

It will be appreciated that detection of the antigen binding protein above a background level is indicative of the presence of La and therefore the presence of dead or dying cancer cells. As such, based on a higher level of antigen binding protein, the skilled person can infer that the treatment has been effective.

Conversely, when detection of the antigen binding protein is the same or below a background level, this may be indicative of the absence of La and therefore the absence of dead or dying cancer cells. As such, based on a same or lower level of antigen binding protein compared to background, the skilled person can infer that the treatment has likely not been effective.

It will also be appreciated that machine learning models and algorithms comprising data from one or more individuals (or indeed from the same individual at an earlier time point prior to or during treatment), can be used to determine whether treatment has been effective or not.

In circumstances where the treatment is determined to be effective, the method may preferably comprise continuing to administer the treatment. In circumstances where the treatment is determine not to have been effective, the method may further comprise discontinuation of the treatment, or increased (eg an increased dose) treatment, or may be supplemented with additional treatments.

Accordingly, the present invention also provides a method of treating a subject with a cytotoxic agent, the method comprising:
administering a cytotoxic agent to a subject, or providing a subject who has been administered a treatment with a cytotoxic agent,
administering an antigen binding protein as described herein to the subject, wherein the antigen binding protein comprises a detectable moiety for enabling detection thereof,
detecting the antigen binding protein in the subject,
determining to continue or continuing to administer the cytotoxic agent to the subject when the detection of the antigen binding protein in the subject is above a background level; or
determining to discontinue or discontinuing to administer the cytotoxic agent to the subject when the detection of the antigen binding protein in the subject is the same or lower than a background level, optionally determining to administer or administering an increased dose of the cytotoxic agent or an alternative or supplemental treatment to the subject.

The detectable moiety may be any moiety which enables detection, preferably in vivo detection, of the antigen binding protein. In certain embodiments, the detectable moiety may be a radionuclide or a fluorescent dye. Preferably, the detectable moiety is a radionuclide, optionally selected from the group consisting of: fluorine-18 (¹⁸F), gallium-67 and gallium-68 (⁶⁷Ga and ⁶⁸Ga), indium-111 (¹¹¹In), iodine-123, -124, (¹²³I, ¹²⁴I) technetium-99 (^{99m}Tc), terbium-155 and terbium-159 (¹⁵⁵Tb and ¹⁵⁹Tb), and zirconium-89 (⁸⁹Zr).

The cytotoxic treatment or cytotoxic agent may be any treatment which induces cell death, such as chemotherapy, radiotherapy (such as external beam radiation therapy (EBRT), ultra-high dose rate (FLASH) radiotherapy, particle radiotherapy sometimes called hadrontherapy, with protons, neutrons or carbon ions), stereotactic ablative body radiotherapy (SABR), immunotherapy (such as immune checkpoint inhibition), allogenic or autologous stem cell transfer (SCT), chemo-immunotherapy or low-dose rate radiotherapy such as brachytherapy or molecular targeted radiation such as radioimmunotherapy or peptide receptor radionuclide therapy.

The subject who has received the cytotoxic treatment may have received the treatment for any condition, although preferably the disease or condition is a cancer.

The present invention therefore also provides use of an antigen binding protein as described herein, in the manufacture of a medicament for:
determining the efficacy of a cytotoxic treatment; and/or
a method of treating a subject with a cytotoxic agent;
   wherein the antigen binding protein comprising a detectable moiety for enabling detection thereof.

The present invention also provides an antigen binding protein or diagnostic composition as described herein for use in:
determining the efficacy of a cytotoxic treatment; and/or
a method of treating a subject with a cytotoxic agent;
   wherein the antigen binding protein comprising a detectable moiety for enabling detection thereof.

In preferred embodiments, the use comprises:
administering an antigen binding protein as described herein to the subject, wherein the antigen binding protein comprises a detectable moiety for enabling detection thereof,
detecting the antigen binding protein in the subject,
determining that the treatment has been effective when the detection of the antigen binding protein in the subject is above a background level;
determining that the treatment has not been effective when the detection of the antigen binding protein in the subject is at the same or at a lower level compared to a background level; and optionally:
determining to continue or continuing to administer the cytotoxic agent to the subject when the detection of the antigen binding protein in the subject is above a background level; or
determining to discontinue or discontinuing to administer the cytotoxic agent to the subject when the detection of the antigen binding protein in the subject is the same or lower than a background level, optionally determining to administer or administering an increased dose of the cytotoxic agent or an alternative or supplemental treatment to the subject.

It will further be appreciated that the antigen binding proteins of the invention can further be used to target surrounding, or treatment-resistant cells in proximity to dead or dying cells following a first cytotoxic therapy. In other words, the antigen binding proteins of the invention, particularly when labelled with a cytotoxic agent, can be used to target and kill viable “bystander cells” that have not been successfully killed by a first treatment.

As such the present invention also provides a method of:
treating, preventing or minimising progression of a disease or condition, preferably of cancer, in a subject,
minimising, reducing or preventing growth of a tumor in a subject,
minimising, reducing or preventing metastasis in a subject, or
increasing survival of a subject,
   the method comprising:
providing a subject who has received a first cytotoxic therapy;
administering to the subject, a second cytotoxic therapy in the form of an antigen binding protein as described herein, wherein the antigen binding protein is conjugated to, or associated with, a cytotoxic agent,
   thereby treating, preventing or minimising progression of a disease or condition, preferably of cancer, in a subject; or minimising, reducing or preventing growth of a tumour in a subject, or minimising, reducing or preventing metastasis in a subject, or increasing survival of a subject

The present invention also provides a combination therapy for treating a disease or condition, preferably cancer, the method comprising:
administering to a subject in need thereof, a first cytotoxic therapy;
administering to the subject, a second cytotoxic therapy in the form of an antigen binding protein as described herein, wherein the antigen binding protein is conjugated to, or associated with, a cytotoxic agent,
   thereby treating a disease or condition, preferably cancer.

In any embodiment, the first cytotoxic therapy may be any treatment which induces cell death, such as chemotherapy, radiotherapy (such as external beam radiation therapy (EBRT)), stereotactic ablative body radiotherapy (SABR), immunotherapy, autologous stem cell transfer (ASCT), chemo-immunotherapy or radioimmunotherapy.

The cytotoxic agent conjugated to the antigen binding protein may be any cytotoxic agent or chemotherapeutic agent.

In preferred embodiments, the cytotoxic agent conjugated to the antigen binding protein is a radionuclide. The radionuclide may be an alpha-emitting, beta-emitting or a gamma-emitting radionuclide. Optionally, the radionuclide is selected from: actinium-225 (²²⁵Ac), astatine-211 (²¹¹At), bismuth-212 and bismuth-213 (²¹²Bi, ²¹³Bi), copper-64 and copper-67 (⁶⁴Cu, ⁶⁷Cu), fluorine-18 (¹⁸F), indium-111 (¹¹¹In), iodine-123, -124, -125 or -131 (¹²³I, ¹²⁴I, ¹²⁵I, ¹³¹I), lead-212 (²¹²Pb), lutetium-177 (¹⁷⁷Lu), radium-223 (²²³Ra), rhenium-186 and rhenium-188 (¹⁸⁶Re, ¹⁸⁸Re), samarium-153 (¹⁵³Sm), scandium-44 and scandium-47 (⁴⁴Sc, ⁴⁷Sc), strontium-90 (⁹⁰Sr), technetium-99 (^{99m}Tc), terbium-155 and terbium-159, yttrium-86 and yttrium-90 (⁸⁶Y, ⁹⁰Y) and zirconium-89 (⁸⁹Zr).

In any embodiment, the first and second cytotoxic therapies may be administered simultaneously, separately or sequentially.

The present invention therefore also provides use of an antigen binding protein as described herein, in the manufacture of a medicament for:
treating, preventing or minimising progression of a disease or condition, preferably of cancer, in a subject,
minimising, reducing or preventing growth of a tumor in a subject,
minimising, reducing or preventing metastasis in a subject, or
increasing survival of a subject,
   wherein the subject has received a cytotoxic therapy.

The present invention therefore also provides use of an antigen binding protein as described herein, in the manufacture of a medicament for use in combination with a cytotoxic therapy, for:
treating, preventing or minimising progression of a disease or condition, preferably of cancer, in a subject,
minimising, reducing or preventing growth of a tumor in a subject,
minimising, reducing or preventing metastasis in a subject, or
increasing survival of a subject.

The cytotoxic therapy may be any treatment which induces cell death, such as chemotherapy, radiotherapy (such as external beam radiation therapy (EBRT)), stereotactic ablative body radiotherapy (SABR), immunotherapy, autologous stem cell transfer (ASCT), chemo-immunotherapy or radioimmunotherapy.

The present invention also provides an antigen binding protein or pharmaceutical composition as described herein for use in:
treating, preventing or minimising progression of a disease or condition, preferably of cancer, in a subject,
minimising, reducing or preventing growth of a tumor in a subject,
minimising, reducing or preventing metastasis in a subject, or
increasing survival of a subject, wherein the subject has received a cytotoxic therapy.

The present invention therefore also provides an antigen binding protein or pharmaceutical composition as described herein, for use in combination with a cytotoxic therapy, for:
treating, preventing or minimising progression of a disease or condition, preferably of cancer, in a subject,
minimising, reducing or preventing growth of a tumor in a subject,
minimising, reducing or preventing metastasis in a subject, or
increasing survival of a subject.

The cytotoxic therapy may be any treatment which induces cell death, such as chemotherapy, radiotherapy (such as external beam radiation therapy (EBRT)), stereotactic ablative body radiotherapy (SABR), immunotherapy, autologous stem cell transfer (ASCT), chemo-immunotherapy or radioimmunotherapy.

In any method of treatment or use for treatment described herein, the antigen binding protein may be conjugated to, or associated with a cytotoxic agent (such as a chemotherapeutic agent or radionuclide).

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1: La N terminal domain ELISA results. Binding of chimeric (legacy and 0/0) and humanised (Legacy=VH0/VK0; 0/0=VH0/VK0; 3/6=VH3/VK6; 6/8=VH6/VK8; 3/6 RadMab=VH3/VK6_K322A_RadMab; and 6/8 RadmAb=VH6; VK8_K322A_RadmAb) antibodies to the N terminal domain of La protein.

FIG. 2: Flow cytometry of antibody binding to human H460 lung cancer cells. A. Flow cytometry images for cells that were untreated (control), treated with cisplatin to induce cell death, or permeabilised with formalin/methanol to expose intracellular La/SSB antigen. B. Mean Fluorescence Intensity (MFI) for each antibody and percentage APOmAB positive cells. (Legacy=VH0/VK0; 0/0=VH0/VK0; 3/6=VH3/VK6; 6/8=VH6/VK8; 3/6 RadmAb=VH3/VK6_K322A_RadmAb; and 6/8 RadmAb=VH6; VK8_K322A_RadmAb).

FIG. 3: Comparison of binding affinities of antibodies following conjugation to DFOAq (A) and following radiolabelling with 89Zr (B).

FIG. 4: In vivo imaging using humanised APOMAB. A. Schematic of experimental outline. B. Exemplary PET scan images on Day 3. C. Tumour uptake derived from PET imaging; i) with K322A mutation; ii) without K322A mutation. D. Biodistribution (tissue update) at Day 3); i) with K322A mutation; ii) without K322A mutation (Legacy=VH0/VK0; 0/0=VH0/VK); 3/6=VH3/VK6; 6/8=VH6/VK8).

FIG. 5: Biodistribution of VH6/VK8 and VH6/VK8 RadmAb. Results show that biostribution of antibody is similar, regardless of the addition of the K322A mutation.

### SEQUENCE INFORMATION

TABLE 1
Sequence information (parental antibody)

| | | SEQ ID | |
|---|---|---|---|
| Anibody | | NO: | Amino acid sequence |
| Parental chimeric heavy variable | | 1 | EVQLQQPGAELVKPGASVKLSCKASGYAFTHYYIY |
| chain (VH) chAPOMAB_VH0 | | | WIKQRPGQGLEWIGGVNPSNGGTHFNEKFKSKATL |
| | | | TVDKSSSTAYMQLSSLTSEDSAVYYCTRSEYDYGL |
| | | | GFAYWGQGTLLTVSS |
| Parental chimeric light variable | | 4 | EIVMSQSPSSLAVSAGEKVTMSCKSSQSLLNSRTPK |
| chain (VL) chAPOMAB_VK0 | | | NYLAWYQQKPGQSPKLLIYWASTRKSGVPDRFTGS |
| | | | GSGTDFTLTISSVQAEDLAVYYCKQSYNLLTFGAGT |
| | | | KLEL |
| chAPOMAB_VH0 | CDRH1 (Kabat) | 7 | HYYIY |
| | CDRH2 (Kabat) | 8 | GVNPSNGGTHFNEKFKS |
| | CDRH3 (Kabat) | 9 | SEYDYGLGFAY |
| | FRH1 (Kabat) | 10 | EVQLQQPGAELVKPGASVKLSCKASGYAFT |
| | FRH2 (Kabat) | 11 | WIKQRPGQGLEWIG |
| | FRH3 (Kabat) | 12 | KATLTVDKSSSTAYMQLSSLTSEDSAVYYCTR |
| | FRH4 (Kabat) | 13 | WGQGTLLTVSS |
| | CDRH1 (IMGT) | 14 | GYAFTHYY |
| | CDRH2 (IMGT) | 15 | VNPSNGGT |
| | CDRH3 (IMGT) | 16 | TRSEYDYGLGFAY |
| | FRH1 (IMGT) | 17 | EVQLQQPGAELVKPGASVKLSCKAS |
| | FRH2 (IMGT) | 18 | IYWIKQRPGQGLEWIGG |
| | FRH3 (IMGT) | 19 | HFNEKFKSKATLTVDKSSSTAYMQLSSLTSEDSAVYYC |
| | FRH4 (IMGT) | 20 | WGQGTLLTVSS |
| chAPOMAB_VK0 | CDRL1 (Kabat) | 49 | KSSQSLLNSRTPKNYLA |
| | CDRL2 (Kabat) | 50 | WASTRKS |
| | CDRL3 (Kabat) | 51 | KQSYNLLT |
| | FRL1 (Kabat) | 52 | EIVMSQSPSSLAVSAGEKVTMSC |
| | FRL2 (Kabat) | 53 | WYQQKPGQSPKLLIY |
| | FRL3 (Kabat) | 54 | GVPDRFTGSGSGTDFTLTISSVQAEDLAVYYC |
| | FRL4 (Kabat) | 55 | FGAGTKLELK |
| | CDRL1 (IMGT) | 56 | QSLLNSRTPKNY |
| | CDRL2 (IMGT) | 57 | WAS |
| | CDRL3 (IMGT) | 58 | KQSYNLLT |
| | FRL1 (IMGT) | 59 | EIVMSQSPSSLAVSAGEKVTMSCKSS |
| | FRL2 (IMGT) | 60 | LAWYQQKPGQSPKLLIY |
| | FRL3 (IMGT) | 61 | TRKSGVPDRFTGSGSGTDFTLTISSVQAEDLAVYYC |
| | FRL4 (IMGT) | 62 | FGAGTKLELK |

TABLE 2
Sequence information (humanised antibodies of the invention)

| | | SEQ ID | |
|---|---|---|---|
| Antibody | Description | NO: | Amino acid sequence |
| Humanised VH | | 2 | EVQLVQSGAEVKKPGASVKVSCKASGYAFTHYYIY |
| hAPOMAB_VH3 | | | WIRQAPGQGLEWMGGVNPSSGGTHYAEKFQGRVT |
| | | | LTVDTSSSTAYMELSSLRSEDTAVYYCTRSEYDYGL |
| | | | GFAYWGQGTLVTVSS |
| Humanised VH | | 3 | EVQLVQSGAEVKKPGASVKVSCKASGYAFTHYYIY |
| hAPOMAB_VH6 | | | WIKQAPGQGLEWIGGVNPSNGGTHFNEKFKSRVTI |
| | | | TVDKSASTAYMELSSLRSEDTAVYYCTRSEYDYGL |
| | | | GFAYWGQGTLVTVSS |
| humanised VL | | 5 | DIVMTQSPLSLPVTPGEPASISCRSSQSLLSSRTPKN |
| hAPOMAB_VK6 | | | YLAWYQQKPGQSPQLLIYWASTRKSGVPDRFSGSG |
| | | | SGTDFTLKISRVEAEDVGVYYCKQSYNLLTFGQGTK |
| | | | LEIK |
| humanised VL | | 6 | EIVMTQSPDSLAVSLGERATINCKSSQSLLNSRTPK |
| hAPOMAB_VK8 | | | NYLAWYQQKPGQPPKLLIYWASTRESGVPDRFTGS |
| | | | GSGTDFTLTISSLQAEDVAVYYCKQSYNLLTFGQGT |
| | | | KLEIK |
| hAPOMAB_VH3 | CDRH1 (Kabat) | 21 | HYYIY |
| | CDRH2 (Kabat) | 22 | GVNPSSGGTHYAEKFQG |
| | CDRH3 (Kabat) | 23 | SEYDYGLGFAY |
| | FRH1 (Kabat) | 24 | EVQLVQSGAEVKKPGASVKVSCKASGYAFT |
| | FRH2 (Kabat) | 25 | WIRQAPGQGLEWMG |
| | FRH3 (Kabat) | 26 | RVTLTVDTSSSTAYMELSSLRSEDTAVYYCTR |
| | FRH4 (Kabat) | 27 | WGQGTLVTVSS |
| | CDRH1 (IMGT) | 28 | GYAFTHYY |
| | CDRH2 (IMGT) | 29 | VNPSSGGT |
| | CDRH3 (IMGT) | 30 | TRSEYDYGLGFAY |
| | FRH1 (IMGT) | 31 | EVQLVQSGAEVKKPGASVKVSCKAS |
| | FRH2 (IMGT) | 32 | IYWIRQAPGQGLEWMGG |
| | FRH3 (IMGT) | 33 | HYAEKFQGRVTLTVDTSSSTAYMELSSLRSEDTAVYYC |
| | FRH4 (IMGT) | 34 | WGQGTLVTVSS |
| hAPOMAB_VH6 | CDRH1 (Kabat) | 35 | HYYIY |
| | CDRH2 (Kabat) | 36 | GVNPSNGGTHFNEKFKS |
| | CDRH3 (Kabat) | 37 | SEYDYGLGFAY |
| | FRH1 (Kabat) | 38 | EVQLVQSGAEVKKPGASVKVSCKASGYAFT |
| | FRH2 (Kabat) | 39 | WIKQAPGQGLEWIG |
| | FRH3 (Kabat) | 40 | RVTITVDKSASTAYMELSSLRSEDTAVYYCTR |
| | FRH4 (Kabat) | 41 | WGQGTLVTVSS |
| | CDRH1 (IMGT) | 42 | GYAFTHYY |
| | CDRH2 (IMGT) | 43 | VNPSNGGT |
| | CDRH3 (IMGT) | 44 | TRSEYDYGLGFAY |
| | FRH1 (IMGT) | 45 | EVQLVQSGAEVKKPGASVKVSCKAS |
| | FRH2 (IMGT) | 46 | IYWIKQAPGQGLEWIGG |
| | FRH3 (IMGT) | 47 | HFNEKFKSRVTITVDKSASTAYMELSSLRSEDTAVYYC |
| | FRH4 (IMGT) | 48 | WGQGTLVTVSS |
| hAPOMAB_VK6 | CDRL1 (Kabat) | 63 | RSSQSLLSSRTPKNYLA |
| | CDRL2 (Kabat) | 64 | WASTRKS |
| | CDRL3 (Kabat) | 65 | KQSYNLLT |
| | FRL1 (Kabat) | 66 | DIVMTQSPLSLPVTPGEPASISC |
| | FRL2 (Kabat) | 67 | WYQQKPGQSPQLLIY |
| | FRL3 (Kabat) | 68 | GVPDRFSGSGSGTDFTLKISRVEAEDVGVYYC |
| | FRL4 (Kabat) | 69 | FGQGTKLEIK |
| | CDRL1 (IMGT) | 70 | QSLLSSRTPKNY |
| | CDRL2 (IMGT) | 71 | WAS |
| | CDRL3 (IMGT) | 72 | KQSYNLLT |
| | FRL1 (IMGT) | 73 | DIVMTQSPLSLPVTPGEPASISCRS |
| | FRL2 (IMGT) | 74 | LAWYQQKPGQSPQLLIY |
| | FRL3 (IMGT) | 75 | TRKSGVPDRFSGSGSGTDFTLKISRVEAEDVGVYYC |
| | FRL4 (IMGT) | 76 | FGQGTKLEIK |
| hAPOMAB_VK8 | CDRL1 (Kabat) | 77 | KSSQSLLNSRTPKNYLA |
| | CDRL2 (Kabat) | 78 | WASTRES |
| | CDRL3 (Kabat) | 79 | KQSYNLLT |
| | FRL1 (Kabat) | 80 | EIVMTQSPDSLAVSLGERATINC |
| | FRL2 (Kabat) | 81 | WYQQKPGQPPKLLIY |
| | FRL3 (Kabat) | 82 | GVPDRFTGSGSGTDFTLTISSLQAEDVAVYYC |
| | FRL4 (Kabat) | 83 | FGQGTKLEIK |
| | CDRL1 (IMGT) | 84 | QSLLNSRTPKNY |
| | CDRL2 (IMGT) | 85 | WAS |
| | CDRL3 (IMGT) | 86 | KQSYNLLT |
| | FRL1 (IMGT) | 87 | EIVMTQSPDSLAVSLGERATINCKSS |
| | FRL2 (IMGT) | 88 | LAWYQQKPGQPPKLLIY |
| | FRL3 (IMGT) | 89 | TRESGVPDRFTGSGSGTDFTLTISSLQAEDVAVYYC |
| | FRL4 (IMGT) | 90 | FGQGTKLEIK |
| Fc constant region (K322A | | 91 | ASTKGPSVFPLAPSSKSTSGGTAALGCLVKDYFPEP |
| mutation and H310A and | | | VTVSWNSGALTSGVHTFPAVLQSSGLYSLSSVVTVP |
| H435Q mutations underlined) | | | SSSLGTQTYICNVNHKPSNTKVDKKVEPKSCDKTHT |
| | | | CPPCPAPELLGGPSVFLFPPKPKDTLMISRTPEVTC |
| | | | VVVDVSHEDPEVKFNWYVDGVEVHNAKTKPREEQ |
| | | | YNSTYRVVSVLTVLAQDWLNGKEYKCAVSNKALPA |
| | | | PIEKTISKAKGQPREPQVYTLPPSREEMTKNQVSLT |
| | | | CLVKGFYPSDIAVEWESNGQPENNYKTTPPVLDSD |
| | | | GSFFLYSKLTVDKSRWQQGNVFSCSVMHEALHNQ |
| | | | YTQKSLSLSPGK |
| hAPOMAB_VH3 + Fc | | 92 | EVQLVQSGAEVKKPGASVKVSCKASGYAFTHYYIY |
| (heavy chain variable + constant | | | WIRQAPGQGLEWMGGVNPSSGGTHYAEKFQGRVT |
| in bold) | | | LTVDTSSSTAYMELSSLRSEDTAVYYCTRSEYDYGL |
| | | | GFAYWGQGTLVTVSSASTKGPSVFPLAPSSKSTSG |
| | | | GTAALGCLVKDYFPEPVTVSWNSGALTSGVHTFPA |
| | | | VLQSSGLYSLSSVVTVPSSSLGTQTYICNVNHKPSN |
| | | | TKVDKKVEPKSCDKTHTCPPCPAPELLGGPSVFLF |
| | | | PPKPKDTLMISRTPEVTCVVVDVSHEDPEVKFNWY |
| | | | VDGVEVHNAKTKPREEQYNSTYRVVSVLTVLAQD |
| | | | WLNGKEYKCAVSNKALPAPIEKTISKAKGQPREPQ |
| | | | VYTLPPSREEMTKNQVSLTCLVKGFYPSDIAVEWE |
| | | | SNGQPENNYKTTPPVLDSDGSFFLYSKLTVDKSRW |
| | | | QQGNVFSCSVMHEALHNQYTQKSLSLSPGK |
| hAPOMAB_VH6 + Fc | | 93 | EVQLVQSGAEVKKPGASVKVSCKASGYAFTHYYIY |
| (heavy chain variable + constant | | | WIKQAPGQGLEWIGGVNPSNGGTHFNEKFKSRVTI |
| chain in bold) | | | TVDKSASTAYMELSSLRSEDTAVYYCTRSEYDYGL |
| | | | GFAYWGQGTLVTVSSASTKGPSVFPLAPSSKSTSG |
| | | | GTAALGCLVKDYFPEPVTVSWNSGALTSGVHTFPA |
| | | | VLQSSGLYSLSSVVTVPSSSLGTQTYICNVNHKPSN |
| | | | TKVDKKVEPKSCDKTHTCPPCPAPELLGGPSVFLF |
| | | | PPKPKDTLMISRTPEVTCVVVDVSHEDPEVKFNWY |
| | | | VDGVEVHNAKTKPREEQYNSTYRVVSVLTVLAQD |
| | | | WLNGKEYKCAVSNKALPAPIEKTISKAKGQPREPQ |
| | | | VYTLPPSREEMTKNQVSLTCLVKGFYPSDIAVEWE |
| | | | SNGQPENNYKTTPPVLDSDGSFFLYSKLTVDKSRW |
| | | | QQGNVFSCSVMHEALHNQYTQKSLSLSPGK |
| hAPOMAB_VK6 (light chain | | 94 | DIVMTQSPLSLPVTPGEPASISCRSSQSLLSSRTPKN |
| variable + constant) | | | YLAWYQQKPGQSPQLLIYWASTRKSGVPDRFSGSG |
| | | | SGTDFTLKISRVEAEDVGVYYCKQSYNLLTFGQGTK |
| | | | LEIKRTVAAPSVFIFPPSDEQLKSGTASVVCLLNNFY |
| | | | PREAKVQWKVDNALQSGNSQESVTEQDSKDSTYS |
| | | | LSSTLTLSKADYEKHKVYACEVTHQGLSSPVTKSF |
| | | | NRGEC |
| hAPOMAB_ABZ_VK8 (light | | 95 | EIVMTQSPDSLAVSLGERATINCKSSQSLLNSRTPK |
| chain + constant) | | | NYLAWYQQKPGQPPKLLIYWASTRESGVPDRFTGS |
| | | | GSGTDFTLTISSLQAEDVAVYYCKQSYNLLTFGQGT |
| | | | KLEIKRTVAAPSVFIFPPSDEQLKSGTASVVCLLNNF |
| | | | YPREAKVQWKVDNALQSGNSQESVTEQDSKDSTY |
| | | | SLSSTLTLSKADYEKHKVYACEVTHQGLSSPVTKSF |
| | | | NRGEC |

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Further aspects of the present invention and further embodiments of the aspects described in the preceding paragraphs will become apparent from the following description, given by way of example and with reference to the accompanying drawings.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

The present inventors have developed humanised antigen binding proteins, for example antibodies, that bind to the Lupus antigen (La or La/SSB). The antigen binding proteins of the invention may have application as diagnostic agents for monitoring cell death during cancer therapy (eg for detecting the presence of dying cancer cells which have exposed La/SSB antigen) or for determining the efficacy of cell therapies which result in exposure of La/SSB antigen. Further, the antigen binding proteins of the invention are believed to have utility as therapeutic agents (eg for killing surround, treatment-resistant cancer cells in proximity to dead or dying cancer cells from which the La/SSB antigen has been exposed).

La/SSB is an antigen that is specific to dead cancer cells and is retained within the tumour allowing ongoing targeting following initial cancer therapy. Critically, it is believed that dying or dead non-cancerous cells have low La/SSB antigen density compared to dead or dying cancer cells. Further, dead cancer cells are cleared inefficiently following chemotherapy, compared to dead “normal” cells after chemotherapy. Thus, without wishing to be bound by theory, the inventors believe that there would be minimal retention of the La-binding proteins of the invention by dead normal cells compared to dead cancer cells.

The inventors further believe that the difference between La/SSB presented by dead normal cells compared to dead cancer cells, and the difference in dead cell clearing after chemotherapy between normal and cancerous cells means that the antigen binding proteins of the invention are particularly amenable to molecular targeted radiation therapy (eg, radioimmunotherapy). This is based on the appreciation that the La/SSB targets in normal cells are not available for binding by a radiolabelled antigen binding protein of the invention, compared to increased availability of La/SSB targets in cancer cells especially following initial cytolytic therapy, resulting in high binding of the radiolabelled antigen binding proteins of the invention, and bystander killing.

The inventors have surprisingly generated humanised antigen binding proteins that bind to Lupus antigen and that display enhanced affinity in vitro compared to chimeric antigen binding proteins for binding to the same target. Without being bound by theory, the inventors believe that this enhanced affinity will lead to an improvement for in vivo therapeutic and diagnostic applications as described herein compared to the chimeric antigen binding protein.

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects, and vice versa, unless the context clearly dictates otherwise. For example, reference to “a” includes a single as well as two or more; reference to “an” includes a single as well as two or more; reference to “the” includes a single as well as two or more and so forth.

Those skilled in the art will appreciate that the present invention is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

One skilled in the art will recognize many methods and materials similar or equivalent to those described herein, which could be used in the practice of the present invention. The present invention is in no way limited to the methods and materials described.

All of the patents and publications referred to herein are incorporated by reference in their entirety.

The present invention is not to be limited in scope by the specific examples described herein, which are intended for the purpose of exemplification only. Functionally-equivalent products, compositions and methods are clearly within the scope of the present invention.

Any example or embodiment of the present invention herein shall be taken to apply mutatis mutandis to any other example or embodiment of the invention unless specifically stated otherwise or the context indicates otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The description and definitions of variable regions and parts thereof, immunoglobulins, antibodies and fragments thereof herein may be further clarified by the discussion in Kabat Sequences of Proteins of Immunological Interest, National Institutes of Health, Bethesda, Md., 1987 and 1991, Bork et al., J Mol. Biol. 242, 309-320, 1994, Chothia and Lesk J. Mol Biol. 196:901-917, 1987, Chothia et al. Nature 342, 877-883, 1989 and/or or Al-Lazikani et al., J Mol Biol 273, 927-948, 1997.

The term “and/or”, e.g., “X and/or Y” shall be understood to mean either “X and Y” or “X or Y” and shall be taken to provide explicit support for both meanings or for either meaning.

As used herein the term “derived from” shall be taken to indicate that a specified integer may be obtained from a particular source albeit not necessarily directly from that source.

Reference herein to a range of, e.g., residues, will be understood to be inclusive. For example, reference to “a region comprising amino acids 56 to 65” will be understood in an inclusive manner, i.e., the region comprises a sequence of amino acids as numbered 56, 57, 58, 59, 60, 61, 62, 63, 64 and 65 in a specified sequence.

### Selected Definitions

As used herein, Lupus antigen, (also referred to as La protein, La/SSB or La antigen), refers to an evolutionarily conserved phosphoprotein of 50 kDa, which under normal conditions accumulated in the nucleus. Phosphorylation of serine-366 of the La protein has been shown to play an essential role in RNA polIII transcript binding activity. Apoptosis dephosphorylation at S366 leads to caspase-mediated cleavage towards the C-terminus to give a 45 kDa truncate that is associated with apoptosis.

The term “isolated protein” or “isolated polypeptide” is a protein or polypeptide that by virtue of its origin or source of derivation is not associated with naturally-associated components that accompany it in its native state; is substantially free of other proteins from the same source. A protein may be rendered substantially free of naturally associated components or substantially purified by isolation, using protein purification techniques known in the art. By “substantially purified” is meant the protein is substantially free of contaminating agents, e.g., at least about 70% or 75% or 80% or 85% or 90% or 95% or 96% or 97% or 98% or 99% free of contaminating agents.

The term “recombinant” shall be understood to mean the product of artificial genetic recombination. Accordingly, in the context of a recombinant protein comprising an antibody antigen binding domain, this term does not encompass an antibody naturally-occurring within a subject's body that is the product of natural recombination that occurs during B cell maturation. However, if such an antibody is isolated, it is to be considered an isolated protein comprising an antibody antigen binding domain. Similarly, if nucleic acid encoding the protein is isolated and expressed using recombinant means, the resulting protein is a recombinant protein comprising an antibody antigen binding domain. A recombinant protein also encompasses a protein expressed by artificial recombinant means when it is within a cell, tissue or subject, e.g., in which it is expressed.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulphide bond. Examples of non-covalent bonds include hydrogen bonds, ionic bonds, Van der Waals forces, and hydrophobic interactions.

The term “polypeptide” or “polypeptide chain” will be understood from the foregoing paragraph to mean a series of contiguous amino acids linked by peptide bonds.

As used herein, the term “antigen binding protein” is used interchangeably with “antigen binding domain” and shall be taken to mean a region or part of an antibody that is capable of specifically binding to an antigen, i.e., a VH or a VL or an Fv comprising both a VH and a VL. The antigen binding domain need not be in the context of an entire antibody, e.g., it can be in isolation (e.g., a domain antibody) or in another form, e.g., as described herein, such as a scFv.

For the purposes for the present disclosure, the term “antibody” includes a protein capable of specifically binding to one or a few closely related antigens by virtue of an antigen binding domain contained within a Fv. This term includes four chain antibodies (e.g., two light chains and two heavy chains), recombinant or modified antibodies (e.g., chimeric antibodies, humanized antibodies, human antibodies, CDR-grafted antibodies, primatized antibodies, de-immunized antibodies, synhumanized antibodies, half-antibodies, bispecific antibodies). An antibody generally comprises constant domains, which can be arranged into a constant region or constant fragment or fragment crystallizable (Fc). Exemplary forms of antibodies comprise a four-chain structure as their basic unit. Full-length antibodies comprise two heavy chains (~50 to 70 kD) covalently linked and two light chains (~23 kDa each). A light chain generally comprises a variable region (if present) and a constant domain and in mammals is either a κ light chain or a λ light chain. A heavy chain generally comprises a variable region and one or two constant domain(s) linked by a hinge region to additional constant domain(s). Heavy chains of mammals are of one of the following types α, δ, ε, γ, or μ. Each light chain is also covalently linked to one of the heavy chains. For example, the two heavy chains and the heavy and light chains are held together by inter-chain disulfide bonds and by non-covalent interactions. The number of inter-chain disulfide bonds can vary among different types of antibodies. Each chain has an N-terminal variable region (VH or VL wherein each are ~110 amino acids in length) and one or more constant domains at the C-terminus. The constant domain of the light chain (CL which is ~110 amino acids in length) is aligned with and disulfide bonded to the first constant domain of the heavy chain (CH1 which is 330 to 440 amino acids in length). The light chain variable region is aligned with the variable region of the heavy chain. The antibody heavy chain can comprise 2 or more additional CH domains (such as, CH2, CH3 and the like) and can comprise a hinge region between the CH1 and CH2 constant domains. Antibodies can be of any type (e.g., IgG, IgE, IgM, IgD, IgA, and IgY), class (e.g., IgG1, IgG2, IgG3, IgG4, IgA1 and IgA2) or subclass. In one example, the antibody is a murine (mouse or rat) antibody or a primate (such as, human) antibody. In one example the antibody heavy chain is missing a C-terminal lysine residue. In one example, the antibody is humanized, synhumanized, chimeric, CDR-grafted or deimmunized.

The terms “full-length antibody”, “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof.

As used herein, “variable region” refers to the portions of the light and/or heavy chains of an antibody as defined herein that is capable of specifically binding to an antigen and, includes amino acid sequences of complementarity determining regions (CDRs); i.e., CDR1, CDR2, and CDR3, and framework regions (FRs). For example, the variable region comprises three or four FRs (e.g., FR1, FR2, FR3 and optionally FR4) together with three CDRs. VH refers to the variable region of the heavy chain. VL refers to the variable region of the light chain.

As used herein, the term “subject” shall be taken to mean any animal including humans, for example a mammal. Exemplary subjects include but are not limited to humans and non-human primates. For example, the subject is a human.

“Antibodies” or “immunoglobulins” or “Igs” are gamma globulin proteins that are found in blood, or other bodily fluids of vertebrates that function in the immune system to bind antigen, hence identifying and neutralizing foreign objects.

Antibodies are generally a heterotetrameric glycoprotein composed of two identical light (L) chains and two identical heavy (H) chains. Each L chain is linked to a H chain by one covalent disulfide bond. The two H chains are linked to each other by one or more disulfide bonds depending on the H chain isotype. Each H and L chain also has regularly spaced intrachain disulfide bridges.

H and L chains define specific Ig domains. More particularly, each H chain has at the N-terminus, a variable domain (VH) followed by three constant domains (CH) for each of the α and γ chains and four CH domains for u and & isotypes. Each L chain has at the N-terminus, a variable domain (V L) followed by a constant domain (CL) at its other end. The VL is aligned with the VH and the CL is aligned with the first constant domain of the heavy chain (CH1).

Antibodies can be assigned to different classes or isotypes. There are five classes of immunoglobulins: IgA, IgD, IgE, IgG, and IgM, having heavy chains designated α, δ, ε, γ, and μ, respectively. The γ and α classes are further divided into subclasses on the basis of relatively minor differences in CH sequence and function, e.g., humans express the following subclasses: IgG1, IgG2, IgG3, IgG4, IgA1, and IgA2. The L chain from any vertebrate species can be assigned to one of two clearly distinct types, called kappa and lambda, based on the amino acid sequences of their constant domains.

The constant domain includes the Fc portion which comprises the carboxy-terminal portions of both H chains held together by disulfides. The effector functions of antibodies such as ADCC are determined by sequences in the Fc region, which region is also the part recognized by Fc receptors (FcR) found on certain types of cells.

The pairing of a VH and VL together forms a “variable region” or “variable domain” including the amino-terminal domains of the heavy or light chain of the antibody. The variable domain of the heavy chain may be referred to as “VH.” The variable domain of the light chain may be referred to as “VL.” The V domain contains an antigen binding protein which affects antigen binding and defines specificity of a particular antibody for its particular antigen. V regions span about 110 amino acid residues and consist of relatively invariant stretches called framework regions (FRs) (generally about 4) of 15-30 amino acids separated by shorter regions of extreme variability called “hypervariable regions” (generally about 3) that are each 9-12 amino acids long. The FRs largely adopt a β-sheet configuration and the hypervariable regions form loops connecting, and in some cases forming part of, the β-sheet structure.

“Hypervariable region”, “HVR”, or “HV” refers to the regions of an antibody variable domain which are hypervariable in sequence and/or form structurally defined loops. Generally, antibodies comprise six hypervariable regions; three in the VH (H1, H2, H3), and three in the VL (L1, L2, L3). A number of hypervariable region delineations are in use and are encompassed herein.

As used herein, the term “complementarity determining regions” (syn. CDRs; i.e., CDR1, CDR2, and CDR3) refers to the amino acid residues of an antibody variable region the presence of which are major contributors to specific antigen binding. Each variable region domain (VH or VL) typically has three CDRs identified as CDR1, CDR2 and CDR3. The CDRs of VH are also referred to herein as CDR H1, CDR H2 and CDR H3, respectively, wherein CDR H1 corresponds to CDR 1 of VH, CDR H2 corresponds to CDR 2 of VH and CDR H3 corresponds to CDR 3 of VH. Likewise, the CDRs of VL are referred to herein as CDR L1, CDR L2 and CDR L3, respectively, wherein CDR L1 corresponds to CDR 1 of VL, CDR L2 corresponds to CDR 2 of VL and CDR L3 corresponds to CDR 3 of VL. In one example, the amino acid positions assigned to CDRs and FRs are defined according to Kabat Sequences of Proteins of Immunological Interest, National Institutes of Health, Bethesda, Md., 1987 and 1991 (also referred to herein as “the Kabat numbering system”). In another example, the amino acid positions assigned to CDRs and FRs are defined according to the Enhanced Chothia Numbering Scheme (http://www.bioinfo.org.uk/mdex.html). The present invention is not limited to FRs and CDRs as defined by the Kabat numbering system, but includes all numbering systems, including the canonical numbering system or of Chothia and Lesk J. Mol. Biol. 196:901-917, 1987; Chothia et al., Nature 342:877-883, 1989; and/or Al-Lazikani et al., J. Mol. Biol. 273:927-948, 1997; the numbering system of Honnegher and Plükthun J. Mol. Biol. 309:657-670, 2001; or the IMGT system discussed in Giudicelli et al., Nucleic Acids Res. 25:206-211 1997. In one example, the CDRs are defined according to the Kabat numbering system. Optionally, heavy chain CDR2 according to the Kabat numbering system does not comprise the five C-terminal amino acids listed herein or any one or more of those amino acids are substituted with another naturally-occurring amino acid. In this regard, Padlan et al., FASEB J., 9:133-139, 1995 established that the five C-terminal amino acids of heavy chain CDR2 are not generally involved in antigen binding.

“Framework” or “FR” residues are those variable domain residues other than the hypervariable region or CDR residues herein defined. The FRs of VH are also referred to herein as FR H1, FR H2, FR H3 and FR H4, respectively, wherein FR H1 corresponds to FR 1 of VH, FR H2 corresponds to FR 2 of VH, FR H3 corresponds to FR 3 of VH and FR H4 corresponds to FR 4 of VH. Likewise, the FRs of VL are referred to herein as FR L1, FR L2, FR L3 and FR L4, respectively, wherein FR L1 corresponds to FR 1 of VL, FR L2 corresponds to FR 2 of VL, FR L3 corresponds to FR 3 of VL and FR L4 corresponds to FR 4 of VL.

“A peptide for forming an antigen binding protein” generally refers to a peptide that may form a conformation that confers the specificity of an antibody for antigen. Examples include whole antibody or whole antibody related structures, whole antibody fragments including a variable domain, variable domains and fragments thereof, including light and heavy chains, or fragments of light and heavy chains that include some but not all of hypervariable regions or constant regions.

An “intact” or “whole” antibody is one which comprises an antigen-binding protein as well as a CL and at least heavy chain constant domains, CH1, CH2 and CH3. The constant domains may be native sequence constant domains (e.g. human native sequence constant domains) or amino acid sequence variant thereof.

“Whole antibody related structures” include multimerized forms of whole antibody.

“Whole antibody fragments including a variable domain” include Fab, Fab′, F(ab′)2, and Fv fragments; diabodies; linear antibodies, single-chain antibody molecules; and multispecific antibodies formed from antibody fragments.

The Fab fragment consists of an entire L chain along with the variable region domain of the H chain (VH), and the first constant domain of one heavy chain (CHI). Each Fab fragment is monovalent with respect to antigen binding, i.e., it has a single antigen-binding protein.

A Fab′ fragment differs from Fab fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group.

A F(ab′)2 fragment roughly corresponds to two disulfide linked Fab fragments having divalent antigen-binding activity and is still capable of cross-linking antigen.

An “Fv” is an antibody fragment which contains a complete antigen-recognition and -binding site. This fragment consists of a dimer of one heavy- and one light-chain variable region domain in tight, non-covalent association.

In a single-chain Fv (scFv) species, one heavy- and one light-chain variable domain can be covalently linked by a flexible peptide linker such that the light and heavy chains can associate in a “dimeric” structure analogous to that in a two-chain Fv species. From the folding of these two domains emanate six hypervariable loops (3 loops each from the H and L chain) that contribute the amino acid residues for antigen binding and confer antigen binding specificity to the antibody.

“Single-chain Fv” also abbreviated as “sFv” or “scFv” are antibody fragments that comprise the VH and VL antibody domains connected to form a single polypeptide chain. Preferably, the scFv polypeptide further comprises a polypeptide linker between the VH and VL domains which enables the scFv to form the desired structure for antigen binding.

A “single variable domain” is half of an Fv (comprising only three CDRs specific for an antigen) that has the ability to recognize and bind antigen, although at a lower affinity than the entire binding site.

“Diabodies” refers to antibody fragments with two antigen-binding sites, which fragments comprise a heavy-chain variable domain (VH) connected to a light-chain variable domain (VL) in the same polypeptide chain (VH-VL). The small antibody fragments are prepared by constructing sFv fragments (see preceding paragraph) with short linkers (about 5-10 residues) between the VH and VL domains such that interchain but not intra-chain pairing of the V domains is achieved, resulting in a bivalent fragment, i.e., fragment having two antigen-binding sites.

Diabodies may be bivalent or bispecific. Bispecific diabodies are heterodimers of two “crossover” sFv fragments in which the VH and VL domains of the two antibodies are present on different polypeptide chains. Triabodies and tetrabodies are also generally known in the art.

An “isolated antibody” is one which has been identified and separated and/or recovered from a component of its pre-existing environment. Contaminant components are materials that would interfere with therapeutic uses for the antibody, and may include enzymes, hormones, and other proteinaceous or nonproteinaceous solutes.

A “human antibody” refers to an antibody which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Human antibodies can be prepared by administering the antigen to a transgenic animal that has been modified to produce such antibodies in response to antigenic challenge, but whose endogenous loci have been disabled.

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin.

“Monoclonal antibody” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site or determinant on the antigen. In addition to their specificity, the monoclonal antibodies are advantageous in that they may be synthesized uncontaminated by other antibodies. Monoclonal antibodies may be prepared by the hybridoma methodology, or may be made using recombinant DNA methods in bacterial, eukaryotic animal or plant cells. The “monoclonal antibodies” may also be isolated from phage antibody libraries.

The monoclonal antibodies herein include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. Chimeric antibodies of interest herein include “primatized” antibodies comprising variable domain antigen-binding sequences derived from a non-human primate (e.g. Old World Monkey, Ape etc), and human constant region sequences.

The term “anti-La antibody” or “an antibody that binds to La” or “La/SSB binding protein or antibody” refers to a protein or an antibody that is capable of binding La with sufficient affinity such that the antibody is useful as a diagnostic and/or therapeutic agent in targeting proteins or cells expressing or presenting La. Preferably, the extent of binding of an anti-La antibody to an unrelated tag or protein is less than about 10% of the binding of the antibody to La as measured, e.g., by a radioimmunoassay (RIA). In certain embodiments, an antibody that binds to La has a dissociation constant (Kd) of <1 μM, <100 nM, <10 nM, <1 nM, or <0.1 nM.

“Binding affinity” generally refers to the strength of the sum total of noncovalent interactions between a single binding site of a molecule (e.g., an antibody) and its binding partner (e.g., an antigen). Generally, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention.

As used herein, the term “binds” in reference to the interaction of an antigen binding protein or an antigen binding domain thereof with an antigen means that the interaction is dependent upon the presence of a particular structure (e.g., an antigenic determinant or epitope) on the antigen. For example, an antibody recognizes and binds to a specific protein structure rather than to proteins generally. If an antibody binds to epitope “A”, the presence of a molecule containing epitope “A” (or free, unlabelled “A”), in a reaction containing labeled “A” and the protein, will reduce the amount of labelled “A” bound to the antibody.

As used herein, the term “specifically binds” or “binds specifically” shall be taken to mean that an antigen binding protein of the invention reacts or associates more frequently, more rapidly, with greater duration and/or with greater affinity with a particular antigen or cell expressing same than it does with alternative antigens or cells.

As used herein, the term “does not detectably bind” shall be understood to mean that an antigen binding protein, e.g., an antibody, binds to a candidate antigen at a level less than 10%, or 8% or 6% or 5% above background. The background can be the level of binding signal detected in the absence of the protein and/or in the presence of a negative control protein (e.g., an isotype control antibody) and/or the level of binding detected in the presence of a negative control antigen. The level of binding is detected using biosensor analysis (e.g. Biacore) in which the antigen binding protein is immobilized and contacted with an antigen.

As used herein, the term “does not significantly bind” shall be understood to mean that the level of binding of an antigen binding protein of the invention to a polypeptide is not statistically significantly higher than background, e.g., the level of binding signal detected in the absence of the antigen binding protein and/or in the presence of a negative control protein (e.g., an isotype control antibody) and/or the level of binding detected in the presence of a negative control polypeptide. The level of binding is detected using biosensor analysis (e.g. Biacore) in which the antigen binding protein is immobilized and contacted with an antigen.

An “affinity matured” antibody is one with one or more alterations in one or more HVRs thereof which result in an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art.

“ADCC” refers to a process called antibody-dependent cellular cytotoxicity, which is an immune response mediated primarily by natural killer (NK) cells in humans. In ADCC, FcγRIII on the surface of an NK cell recognizes the Fe region of antibody that is bound to antigen displayed on the surface of a target cell. This activates the NK cell, which releases perforins and granzymes, leading to lysis and apoptosis of the target cells.

“CDC” refers to a complex process called complement-dependent cytotoxicity that can lead to cell killing through the action of a cascade of proteins that can act through either of two major pathways.

“ADCP” refers to a process called antibody dependent cell-mediated phagocytosis. In this Fe receptor-mediated process, target cells to which antibodies are bound are engulfed by phagocytic cells, such as macrophage, monocytes, neutrophils, and dendritic cells. Multiple Fc receptors are involved in this process.

A “blocking” antibody or an “antagonist” antibody is one which inhibits or reduces biological activity of the antigen it binds. Preferred blocking antibodies or antagonist antibodies substantially or completely inhibit the biological activity of the antigen.

An “agonist antibody”, as used herein, is an antibody which mimics at least one of the functional activities of a polypeptide of interest.

As meant herein, an “Fc region” is a dimer consisting of two polypeptide chains joined by one or more disulfide bonds, each chain comprising part or all of a hinge domain plus a CH2 and a CH3 domain. Each of the polypeptide chains is referred to as an “Fc polypeptide chain.” To distinguish the two Fe polypeptide chains, one is referred to herein as an “A chain” and the other is referred to as a “B chain.” More specifically, the Fc regions contemplated for use with the present invention are IgG Fc regions, which can be mammalian or human IgG1, IgG2, IgG3, or IgG4 Fc regions. Among human IgG1 Fc regions, at least two allelic types are known.

An “Fc-containing protein,” as meant herein, is a protein comprising an Fc region as described herein and a binding region that binds to a target molecule. The term “Fc containing protein” encompasses an antibody or an Fc fusion protein that contains an Fc region.

The phrase “therapeutically effective amount” generally refers to an amount of an antigen binding protein of the present invention that (i) treats the particular disease, condition, or disorder, (ii) attenuates, ameliorates, or eliminates one or more symptoms of the particular disease, condition, or disorder, or (iii) delays the onset of one or more symptoms of the particular disease, condition, or disorder described herein.

The words “treat” or “treatment” refer to therapeutic treatment wherein the object is to slow down (lessen) an undesired physiological change or disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. Treatment can also mean prolonging survival as compared to expected survival if not receiving treatment. Treatment may not necessarily result in the complete clearance of a disease or disorder but may reduce or minimise complications and side effects of infection and the progression of a disease or disorder. The success or otherwise of treatment may be monitored by, amongst other things, physical examination of the individual, cytopathological, serological DNA, or mRNA detection techniques.

The words “prevent” and “prevention” generally refer to prophylactic or preventative measures for protecting or precluding an individual not having a given disease or disorder from progressing to that disease or disorder.

The phrase “pharmaceutically acceptable” indicates that the substance or composition must be compatible chemically and/or toxicologically, with the other ingredients comprising a formulation, and/or the mammal being treated therewith.

### Mutations to Proteins

The present invention also provides an antigen binding protein or a nucleic acid encoding same having at least 80% identity to a sequence disclosed herein. In one example, an antigen binding protein or nucleic acid of the invention comprises sequence at least about 85% or 90% or 95% or 97% or 98% or 99% identical to a sequence disclosed herein.

Alternatively, or additionally, the antigen binding protein comprises a CDR (e.g., three CDRs) at least about 80% or 85% or 90% or 95% or 97% or 98% or 99% identical to CDR(s) of a VH or VL as described herein according to any example.

In another example, a nucleic acid of the invention comprises a sequence at least about 80% or 85% or 90% or 95% or 97% or 98% or 99% identical to a sequence encoding an antigen binding protein having a function as described herein according to any example. The present invention also encompasses nucleic acids encoding an antigen binding protein of the invention, which differs from a sequence exemplified herein as a result of degeneracy of the genetic code.

The % identity of a nucleic acid or polypeptide is determined by GAP (Needleman and Wunsch. Mol. Biol. 48, 443-453, 1970) analysis (GCG program) with a gap creation penalty=5, and a gap extension penalty=0.3. The query sequence is at least 50 residues in length, and the GAP analysis aligns the two sequences over a region of at least 50 residues. For example, the query sequence is at least 100 residues in length and the GAP analysis aligns the two sequences over a region of at least 100 residues. For example, the two sequences are aligned over their entire length.

The present invention also contemplates a nucleic acid that hybridizes under stringent hybridization conditions to a nucleic acid encoding an antigen binding protein described herein. A “moderate stringency” is defined herein as being a hybridization and/or washing carried out in 2×SSC buffer, 0.1% (w/v) SDS at a temperature in the range 45° C. to 65° C., or equivalent conditions. A “high stringency” is defined herein as being a hybridization and/or wash carried out in 0.1×SSC buffer, 0.1% (w/v) SDS, or lower salt concentration, and at a temperature of at least 65° C., or equivalent conditions. Reference herein to a particular level of stringency encompasses equivalent conditions using wash/hybridization solutions other than SSC known to those skilled in the art. For example, methods for calculating the temperature at which the strands of a double stranded nucleic acid will dissociate (also known as melting temperature, or Tm) are known in the art. A temperature that is similar to (e.g., within 5° C. or within 10° C.) or equal to the Tm of a nucleic acid is considered to be high stringency. Medium stringency is to be considered to be within 10° C. to 20° C. or 10° C. to 15° C. of the calculated Tm of the nucleic acid.

The present invention also contemplates mutant forms of an antigen binding protein of the invention comprising one or more conservative amino acid substitutions compared to a sequence set forth herein. In some examples, the antigen binding protein comprises 10 or fewer, e.g., 9 or 8 or 7 or 6 or 5 or 4 or 3 or 2 or 1 conservative amino acid substitutions. A “conservative amino acid substitution” is one in which the amino acid residue is replaced with an amino acid residue having a similar side chain and/or hydropathicity and/or hydrophilicity.

Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), β-branched side chains (e.g., threonine, valine, isoleucine) and aromatic side chains (e.g., tyrosine, phenylalanine, tryptophan, histidine). Hydropathic indices are described, for example in Kyte and Doolittle J. Mol. Biol., 157:105-132, 1982 and hydrophylic indices are described in, e.g., U.S. Pat. No. 4,554,101.

The present invention also contemplates non-conservative amino acid changes. For example, of particular interest are substitutions of charged amino acids with another charged amino acid and with neutral or positively charged amino acids. In some examples, the antigen binding protein comprises 10 or fewer, e.g., 9 or 8 or 7 or 6 or 5 or 4 or 3 or 2 or 1 non-conservative amino acid substitutions.

In one example, the mutation(s) occur within a FR of an antigen binding domain of an antigen binding protein of the invention. In another example, the mutation(s) occur within a CDR of an antigen binding protein of the invention.

Exemplary methods for producing mutant forms of an antigen binding protein include:
mutagenesis of DNA (Thie et al., Methods Mol. Biol. 525:309-322, 2009) or RNA (Kopsidas et al., Immunol. Lett. 107:163-168, 2006; Kopsidas et al. BMC Biotechnology, 7:18, 2007; and WO1999/058661);
introducing a nucleic acid encoding the polypeptide into a mutator cell, e.g., XL-1Red, XL-mutS and XL-mutS-Kanr bacterial cells (Stratagene);
DNA shuffling, e.g., as disclosed in Stemmer, Nature 370:389-91, 1994; and
site directed mutagenesis, e.g., as described in Dieffenbach (ed) and Dveksler (ed) (In: PCR Primer: A Laboratory Manual, Cold Spring Harbor Laboratories, NY, 1995).

Exemplary methods for determining biological activity of the mutant antigen binding proteins of the invention will be apparent to the skilled artisan and/or described herein, e.g., antigen binding. For example, methods for determining antigen binding, competitive inhibition of binding, affinity, association, dissociation and therapeutic efficacy are described herein.

As used herein, the properties of amino acids are defined in the following table:

| Amino Acid | 3-letter code | 1-letter code | Properties |
|---|---|---|---|
| Alanine | Ala | A | aliphatic |
| | | | hydrophobic |
| | | | neutral |
| Arginine | Arg | R | polar |
| | | | hydrophilic |
| | | | charged (+) |
| Asparagine | Asn | N | polar |
| | | | hydrophilic |
| | | | neutral |
| Aspartate | Asp | D | polar |
| | | | hydrophilic |
| | | | charged (−) |
| Cysteine | Cys | C | polar |
| | | | hydrophobic |
| | | | neutral |
| Glutamine | Gln | Q | polar |
| | | | hydrophilic |
| | | | neutral |
| Glutamate | Glu | E | polar |
| | | | hydrophilic |
| | | | charged (−) |
| Glycine | Gly | G | aliphatic |
| | | | neutral |
| Histidine | His | H | aromatic |
| | | | polar |
| | | | hydrophilic |
| | | | charged (+) |
| Isoleucine | Ile | I | aliphatic |
| | | | hydrophobic |
| | | | neutral |
| Leucine | Leu | L | aliphatic |
| | | | hydrophobic |
| | | | neutral |
| Lysine | Lys | K | polar |
| | | | hydrophilic |
| | | | charged (+) |
| Methionine | Met | M | hydrophobic |
| | | | neutral |
| Phenylalanine | Phe | F | aromatic |
| | | | hydrophobic |
| | | | neutral |
| Proline | Pro | P | hydrophobic |
| | | | neutral |
| Serine | Ser | S | polar |
| | | | hydrophilic |
| | | | neutral |
| Threonine | Thr | T | polar |
| | | | hydrophilic |
| | | | neutral |
| Tryptophan | Trp | W | aromatic |
| | | | hydrophobic |
| | | | neutral |
| Tyrosine | Tyr | Y | aromatic |
| | | | polar |
| | | | hydrophobic |
| Valine | Val | V | aliphatic |
| | | | hydrophobic |
| | | | neutral |

### Constant Regions

The present invention encompasses antigen binding proteins and/or antibodies described herein comprising a constant region of an antibody. This includes antigen binding fragments of an antibody fused to an Fc.

Sequences of constant regions useful for producing the proteins of the present invention may be obtained from a number of different sources. In some examples, the constant region or portion thereof of the protein is derived from a human antibody. The constant region or portion thereof may be derived from any antibody class, including IgM, IgG, IgD, IgA and IgE, and any antibody isotype, including IgG1, IgG2, IgG3 and IgG4. In one example, the constant region is human isotype IgG4 or a stabilized IgG4 constant region.

In various embodiments of the invention, the Fc region of the antibody may comprise one or more substitutions for altering effector function (including increasing or decreasing effector functions), and circulation half-life. Various examples of such substitutions and modifications are described in Saunders (2019) Front. Immunol. article 1296, incorporated herein by reference in its entirety.

In one example, the Fc region of the constant region has a reduced ability to induce effector function, e.g., compared to a native or wild-type human IgG1 or IgG3 Fc region. In one example, the effector function is antibody-dependent cell-mediated cytotoxicity (ADCC) and/or antibody-dependent cell-mediated phagocytosis (ADCP) and/or complement-dependent cytotoxicity (CDC). Methods for assessing the level of effector function of an Fc region containing protein are known in the art and/or described herein.

In one example, the Fc region is an IgG4 Fc region (i.e., from an IgG4 constant region), e.g., a human IgG4 Fc region. Sequences of suitable IgG4 Fc regions will be apparent to the skilled person and/or available in publically available databases (e.g., available from National Center for Biotechnology Information).

In one example, the constant region is a stabilized IgG4 constant region. The term “stabilized IgG4 constant region” will be understood to mean an IgG4 constant region that has been modified to reduce Fab arm exchange or the propensity to undergo Fab arm exchange or formation of a half-antibody or a propensity to form a half antibody. “Fab arm exchange” refers to a type of protein modification for human IgG4, in which an IgG4 heavy chain and attached light chain (half-molecule) is swapped for a heavy-light chain pair from another IgG4 molecule. Thus, IgG4 molecules may acquire two distinct Fab arms recognizing two distinct antigens (resulting in bispecific molecules). Fab arm exchange occurs naturally in vivo and can be induced in vitro by purified blood cells or reducing agents such as reduced glutathione. A “half antibody” forms when an IgG4 antibody dissociates to form two molecules each containing a single heavy chain and a single light chain.

In one example, a stabilized IgG4 constant region comprises a proline at position 241 of the hinge region according to the system of Kabat (Kabat et al., Sequences of Proteins of Immunological Interest Washington DC United States Department of Health and Human Services, 1987 and/or 1991). This position corresponds to position 228 of the hinge region according to the EU numbering system (Kabat et al., Sequences of Proteins of Immunological Interest Washington DC United States Department of Health and Human Services, 2001 and Edelman et al., Proc. Natl. Acad. USA, 63, 78-85, 1969). In human IgG4, this residue is generally a serine. Following substitution of the serine for proline, the IgG4 hinge region comprises a sequence CPPC. In this regard, the skilled person will be aware that the “hinge region” is a proline-rich portion of an antibody heavy chain constant region that links the Fc and Fab regions that confers mobility on the two Fab arms of an antibody. The hinge region includes cysteine residues which are involved in inter-heavy chain disulfide bonds. It is generally defined as stretching from Glu226 to Pro243 of human IgG1 according to the numbering system of Kabat. Hinge regions of other IgG isotypes may be aligned with the IgG1 sequence by placing the first and last cysteine residues forming inter-heavy chain disulphide (S—S) bonds in the same positions (see for example WO2010/080538).

Additional examples of stabilized IgG4 antibodies are antibodies in which arginine at position 409 in a heavy chain constant region of human IgG4 (according to the EU numbering system) is substituted with lysine, threonine, methionine, or leucine (e.g., as described in WO2006/033386). The Fc region of the constant region may additionally or alternatively comprise a residue selected from the group consisting of: alanine, valine, glycine, isoleucine and leucine at the position corresponding to 405 (according to the EU numbering system). Optionally, the hinge region comprises a proline at position 241 (i.e., a CPPC sequence) (as described above).

In another example, the Fc region is a region modified to have reduced effector function, i.e., a “non-immunostimulatory Fc region”. For example, the Fc region is an IgG1 Fc region comprising a substitution at one or more positions selected from the group consisting of 268, 309, 330 and 331. In another example, the Fc region is an IgG1 Fc region comprising one or more of the following changes E233P, L234V, L235A and deletion of G236 and/or one or more of the following changes A327G, A330S and P331S (Armour et al., Eur J Immunol. 29:2613-2624, 1999; Shields et al., J Biol Chem. 276 (9): 6591-604, 2001). Additional examples of non-immunostimulatory Fc regions are described, for example, in Dall'Acqua et al., J Immunol. 177:1129-1138 2006; and/or Hezareh J Virol; 75:12161-12168, 2001).

Antibodies with reduced effector function include those with substitution of one or more of Fc region residues 238, 265, 269, 270, 297, 327 and 329 (as described in U.S. Pat. No. 6,737,056, incorporated herein by reference). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581). For example, an antibody variant may comprise an Fc region with one or more amino acid substitutions which diminish FcγR binding, e.g., substitutions at positions 234 and 235 of the Fc region (EU numbering of residues). For example, the substitutions are L234A and L235A (LALA) (See, e.g., WO 2012/130831). The substitutions may additionally include substitution of the proline residue at position 329, such as a P329G mutation to disable binding to FcR. Further, alterations may be made in the Fc region that result in altered (i.e., diminished) C1q binding and/or Complement Dependent Cytotoxicity (CDC), e.g., as described in U.S. Pat. No. 6,194,551, WO 99/51642, and Idusogie et al. J. Immunol. 164:4178-4184 (2000).

In some aspects, the Fc region includes mutations to the complement (C1q) and/or to Fc gamma receptor (FcγR) binding sites. In some aspects, such mutations can render the antibody incapable of antibody directed cytotoxicity (ADCC) and complement directed cytotoxicity (CDC). One example of a CDC-deficient antibody is one that comprises a substitution at one or more of Glu318, Lys320, Pro 329, Pro331 and Lys322 (for example, K322A), wherein the numbering of the residues in the Fc region is according to the EU index as described in Kabat et al.

In another example, the Fc region is a chimeric Fc region, e.g., comprising at least one CH2 domain from an IgG4 antibody and at least one CH3 domain from an IgG1 antibody, wherein the Fc region comprises a substitution at one or more amino acid positions selected from the group consisting of 240, 262, 264, 266, 297, 299, 307, 309, 323, 399, 409 and 427 (EU numbering) (e.g., as described in WO2010/085682). Exemplary substitutions include 240F, 262L, 264T, 266F, 297Q, 299A, 299K, 307P, 309K, 309M, 309P, 323F, 399S, and 427F.

### Additional Modifications

The present invention also contemplates additional modifications to an antibody or antigen binding protein comprising an Fc region or constant region.

The neonatal Fc-receptor (FcRn) is important for the metabolic fate of antibodies of the IgG class in vivo. The FcRn functions to salvage IgG from the lysosomal degradation pathway, resulting in reduced clearance and increased half-life. FcRn binds with high affinity to the CH2-CH3 portion of the Fc-region of an antibody of the class IgG. The interaction between an antibody of the class IgG and the FcRn is pH dependent and occurs in a 1:2 stoichiometry, i.e. one IgG antibody molecule can interact with two FcRn molecules via its two heavy chain Fc-region polypeptides (see e.g. Huber, A. H., et al, J. Mol. Biol. 230 (1993) 1077-1083).

In certain embodiments of the invention, an antibody may comprise one or more amino acid substitutions that increase the half-life of the protein. For example, the antibody comprises a Fc region comprising one or more amino acid substitutions that increase the affinity of the Fc region for the neonatal Fc region (FcRn). For example, the Fc region has increased affinity for FcRn at lower pH, e.g., about pH 6.0, to facilitate Fc/FcRn binding in an endosome. In one example, the Fc region has increased affinity for FcRn at about pH 6 compared to its affinity at about pH 7.4, which facilitates the re-release of Fc into blood following cellular recycling. These amino acid substitutions are useful for extending the half-life of a protein, by reducing clearance from the blood.

Exemplary amino acid substitutions include T250Q and/or M428L or T252A, T254S and T266F or M252Y, S254T and T256E or H433K and N434F according to the EU numbering system. Additional or alternative amino acid substitutions are described, for example, in US20070135620 or U.S. Pat. No. 7,083,784.

In further embodiments, the antibody comprises one or more amino acid substitutions that decrease the half-life of the protein. For example, the antibody comprises a Fc region comprising one or more amino acid substitutions that decrease or reduce the affinity of the Fc region for the neonatal Fc region (FcRn).

The present invention therefore provides for an antibody having substitutions in the CH2 and/or CH3 domains of the constant region and comprising substitutions at one or more of residues His310, His435, His436 and Ile253 (Kabat numbering), thereby altering FcRn binding affinity and/or serum half-life of said antibody relative to a naturally occurring antibody.

In some examples, the amino acid at position 310 and/or 435 of the antibody may be alanine, glutamic acid, aspartic acid, leucine, isoleucine, arginine, proline, glutamine, methionine, serine, threonine, lysine, asparagine, phenylalanine, tyrosine, tryptophan, cysteine, valine or glycine.

Preferably, the residue at position 310 is selected from alanine, or glutamic acid or glutamine; or amino acid residue 435 from the heavy chain constant region is selected from arginine, glutamine or alanine. In other preferred embodiments, the antibody has an alanine residue at position 310 and glutamine residue at position 435.

In a preferred embodiment of the present invention, the binding affinity for FcRn and/or the serum half-life of the modified antibody is decreased by at least about 2-fold, 3-fold, 4-fold, 5-fold, 10-fold, 15-fold, 20-fold, 25-fold, 30-fold, 40-fold, 50-fold, 60-fold, 70-fold, 80-fold, 90-fold, or 100-fold. In a preferred embodiment of the present invention, the binding affinity for FcRn and/or the serum half-life of said modified antibody is reduced by at least about 20%, 30%, 40%, 50%, 60%, 70%, 80%, 85%, 90%, 95%, 97%, 98%, or 99%.

The antibody may also comprise amino acid substitutions at residues equivalent to Ser228 and Leu235 of the constant heavy chain region, such as Ser228Pro amd/or Leu235Glu.

### Antibody Binding Domain Containing Proteins

In another embodiment there is provided an antigen binding protein as described above wherein an amino acid sequence forming one or more of FR1, CDR1, FR2, CDR2, FR3, CDR3 and FR4 is derived from a human sequence or in the form of a human sequence.

The antigen binding protein may be presented in a humanized form including non-human (e.g., murine) and human immunoglobulin sequences. Typically all but the CDR sequences of the antigen binding protein are from a non-human species such as mouse, rat or rabbit. In some instances, framework residues of the antigen binding protein may also be non-human. Where the antigen binding protein is provided in the form of a whole antibody, typically at least a portion of an immunoglobulin constant region (Fc) is human, thereby allowing various human effector functions.

Methods for humanizing non-human antigen binding proteins are well known in the art, examples of suitable processes including those in Jones et al., (1986) Nature, 321:522; Riechmann et al., (1988) Nature, 332:323; Verhoeyen et al., (1988) Science, 239:1534.

Variable domains including CDRs and FRs of the invention may have been made less immunogenic by replacing surface-exposed residues so as to make the antibody appear as self to the immune system. Padlan, E. A., 1991, Mol. Immunol. 28, 489 provides an exemplary method. Generally, affinity is preserved because the internal packing of amino acid residues in the vicinity of the antigen binding protein remains unchanged and generally CDR residues or adjacent residues which influence binding characteristics are not to be substituted in these processes.

In another embodiment there is provided an anti-La binding protein, immunoglobulin variable domain, antibody, dab, scFv, Fab, Fab′, F(ab′)2, Fv fragment, diabody, triabody, linear antibody, single-chain antibody molecule, or multispecific antibody as described herein, preferably with a sequence as shown in Table 2.

In certain embodiments, the antigen binding protein is provided in the form of a single chain Fv fragment (scFv). Fv and scFv are suitable for reduced nonspecific binding during in vivo use as they have intact combining sites that are devoid of constant regions. Fusion proteins including scFv may be constructed to yield fusion of an effector protein at either the amino or the carboxy terminus of an scFv.

In another embodiment there is provided a diabody or triabody or other multispecific antibody including an antigen binding protein as described above. Multispecific antibodies may be assembled using polypeptide domains that allow for multimerization. Examples include the CH2 and CH3 regions of the Fc and the CH1 and Ckappa/lambda regions. Other naturally occurring protein multimerization domains may be used including leucine zipper domain (bZIP), helix-loop-helix motif, Src homology domain (SH2, SH3), an EF hand, a phosphotyrosine binding (PTB) domain, or other domains known in the art.

In another embodiment there is provided a fusion domain or heterologous protein including an antigen binding protein, immunoglobulin variable domain, antibody, dab, scFv, Fab, Fab′, F(ab′)2, Fv fragment, diabody, triabody, linear antibody, single-chain antibody molecule, or multispecific antibody as described herein. The antigen binding protein may be fused to one or more domains with separate activities.

A heterologous polypeptide may be recombinantly fused or chemically conjugated, for example, to an N- or C-terminus of an antigen binding protein or molecule containing same of the invention.

Further, the antigen binding protein, immunoglobulin variable domain, antibody, dab, scFv, Fab, Fab′, F(ab′)2, Fv fragment, diabody, triabody, linear antibody, single-chain antibody molecule, or multispecific antibody of the invention may be modified by glycosylation, acetylation, pegylation, phosphorylation, amidation, derivatization by known protecting/blocking groups, proteolytic cleavage, linkage to a cellular ligand or other protein, etc.

### Further Details of Antigen Binding Proteins of the Invention

Antigen binding proteins of the invention can be composed of amino acids joined to each other by peptide bonds or modified peptide bonds, i.e., peptide isosteres, and may contain amino acids other than the 20 gene-encoded amino acids. Antigen binding proteins of the invention may be modified by natural processes, such as posttranslational processing, or by chemical modification techniques which are well known in the art. Such modifications are well described in basic texts, as well as in research literature. Modifications can occur anywhere in the antigen binding protein, including the peptide backbone, the amino acid side-chains and the amino or carboxyl termini, or on moieties such as carbohydrates. It will be appreciated that the same type of modification may be present in the same or varying degrees at several proteins in a given antigen binding protein. Also, a given antigen binding protein may contain many types of modifications. An antigen binding protein may be branched, for example, as a result of ubiquitination, and they may be cyclic, with or without branching. Cyclic, branched, and branched cyclic antigen binding proteins may result from post-translation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination.

In another embodiment there is provided a conjugate in the form of an antigen binding protein, immunoglobulin variable domain, antibody, Fab, dab, scFv, diabody, triabody or fusion protein as described above conjugated to a cytotoxic agent such as a chemo therapeutic agent, a drug, a growth inhibitory agent, a toxin (e.g., an enzymatically active toxin of bacterial, fungal, plant, or animal origin, or fragments thereof), or a label such as a radioactive isotope (i.e., a radio conjugate). In another aspect, the invention further provides methods of using the immunoconjugates. In one aspect, an immunoconjugate comprises any of the above variable domains covalently attached to a cytotoxic agent or a detectable agent. In another embodiment, the antigen binding domain is associated with a cytotoxic agent. For example, an antigen binding protein described herein may be expressed on the surface of a cytotoxic cell.

In another embodiment there is provided an antibody for binding to an antigen binding protein, immunoglobulin variable domain, antibody, dab, scFv, Fab, Fab′, F(ab′)2, Fv fragment, diabody, triabody, linear antibody, single-chain antibody molecule, or multispecific antibody, fusion protein or conjugate as described above.

In another embodiment there is provided a nucleic acid encoding an antigen binding protein, immunoglobulin variable domain, antibody, dab, scFv, Fab, Fab′, F(ab′)2, Fv fragment, diabody, triabody, linear antibody, single-chain antibody molecule, or multispecific antibody, fusion protein or conjugate as described above.

A polynucleotide encoding an CDR or FR according to any one of the general formulae described above, or an antigen binding protein comprised of same, may be generated from a nucleic acid from any source, for example by chemical synthesis or isolation from a cDNA or genomic library. For example a cDNA library may be generated from an antibody producing cell such as a B cell, plasma cell or hybridoma cell and the relevant nucleic acid isolated by PCR amplification using oligonucleotides directed to the particular clone of interest. Isolated nucleic acids may then be cloned into vectors using any method known in the art. The relevant nucleotide sequence may then be mutagenized using methods known in the art e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N. Y. and Ausubel et al., eds., 1998, Current Protocols in Molecular Biology, John Wiley & Sons, NY), to generate antigen binding proteins having a different amino acid sequence, for example to create amino acid substitutions, deletions, and/or insertions.

### Protein Production

In another embodiment there is provided a method for producing an anti-La antigen binding protein as described above including expressing a nucleic acid as described above in a cell or non-human animal as described above.

The production of an antigen binding protein of the invention generally requires an expression vector containing a polynucleotide that encodes the antigen binding protein of the invention. A polynucleotide encoding an antigen binding protein of the invention may be obtained and sub cloned into a vector for the production of an antigen binding protein by recombinant DNA technology using techniques well-known in the art, including techniques described herein. Many different expression systems are contemplated including the use of mammalian cells including human cells for production and secretion of antigen binding proteins. Examples of cells include 293F, CHO and the NSO cell line.

Expression vectors containing protein coding sequences and appropriate transcriptional and translational control signals can be constructed using methods known in the art. These include in vitro recombinant DNA techniques, synthetic techniques and in vivo genetic recombination. In certain embodiments there is provided a replicable vector having a nucleic acid encoding an antigen binding protein operably linked to a promoter.

Cells transfected with an expression vector may be cultured by conventional techniques to produce an antigen binding protein. Thus, in certain embodiments, there is provided host cells or cell transfectants containing a polynucleotide encoding an antigen binding protein of the invention operably linked to a promoter. The promoter may be heterologous. A variety of host-expression vector systems may be utilized and in certain systems the transcription machinery of the vector system is particularly matched to the host cell. For example, mammalian cells such as Chinese hamster ovary cells (CHO) may be transfected with a vector including the major intermediate early gene promoter element from human cytomegalovirus. Additionally or alternatively, a host cell may be used that modulates the expression of inserted sequences, or modifies and processes the gene product as required, including various forms of post translational modification. Examples of mammalian host cells having particular post translation modification processes include CHO, VERY, BHK, Hela, COS, MDCK, 293, 3T3, W138, BT483, Hs578T, HTB2, BT20 and T47D, NSO, CRL7030 and HsS78Bst cells.

Depending upon the use intended for the protein molecule, a number of bacterial expression vectors may be advantageously selected. In one example, vectors that cause the expression of high levels of fusion protein products that are readily purified, such as the E. coli expression vector pUR278 may be used where a large quantity of an antigen binding protein is to be produced. The expression product may be produced in the form of a fusion protein with lacZ. Other bacterial vectors include pIN vectors and the like. pGEX vectors may also be used to express foreign polypeptides as fusion proteins with glutathione-S-transferase (GST). These fusion proteins are generally soluble and can easily be purified from lysed cells by adsorption and binding to glutathione-agarose affinity matrix followed by elution in the presence of free glutathione. A thrombin and/or factor Xa protease cleavage site may be provided in the expressed polypeptide so that the cloned target gene product can be released from the GST moiety.

Autographa californica nuclear polyhedrosis virus (AcNPV) may be used as a vector to express foreign genes in an insect system including Spodoptera frugiperda cells. The particular promoter used may depend on where the protein coding is inserted into the sequence. For example, the sequence may be cloned individually into the polyhedrin gene and placed under control of the polyhedrin promoter.

Virus based expression systems may be utilized with mammalian cells such as an adenovirus whereby the coding sequence of interest may be ligated to the adenoviral late promoter and tripartite leader sequence. In vitro or in vivo recombination may then be used to insert this chimeric gene into the adenoviral genome. Insertions into region E1 or E3 will result in a viable recombinant virus that is capable of expressing the antigen binding protein in infected host cells. Specific initiation signals including the ATG initiation codon and adjacent sequences may be required for efficient translation of inserted antigen binding protein coding sequences. Initiation and translational control signals and codons can be obtained from a variety of origins, both natural and synthetic. Transcription enhancer elements and transcription terminators may be used to enhance the efficiency of expression of a viral based system.

Where long-term, high-yield production of recombinant proteins is required, stable expression is preferred. Generally a selectable marker gene is used whereby following transfection, cells are grown for 1-2 days in an enriched media and then transferred to a medium containing a selective medium in which cells containing the corresponding selectable marker, for example, antibiotic resistance can be screened. The result is that cells that have stably integrated the plasmid into their chromosomes grow and form foci that in turn can be cloned and expanded into cell lines. The herpes simplex virus thymidine kinase, hypoxanthineguanine phosphoribosyltransferase and adenine phosphoribosyltransferase genes are examples of genes that can be employed in tk-, hgprt- or aprT- cells, respectively, thereby providing appropriate selection systems. The following genes: dhfr, which confers resistance to methotrexate; gpt, which confers resistance to mycophenolic acid; neo, which confers resistance to the aminoglycoside G-418; and hygro, which confers resistance to hygromycin are examples of genes that can be used in anti-metabolite selection systems.

An antigen binding protein of the invention may be purified by a recombinant expression system by known methods including ion exchange chromatography, affinity chromatography (especially affinity for the specific antigens Protein A or Protein G) and gel filtration column chromatography), centrifugation, differential solubility, or by any other standard technique for the purification of proteins. Purification may be facilitated or assisted by providing the antigen binding protein in the form of a fusion protein.

Large quantities of the antigen binding proteins of the invention may be produced by a scalable process starting with a pilot expression system in a research laboratory that is scaled up to an analytical scale bioreactor (typically from 5 L to about 50 L bioreactors) or production scale bioreactors (for example, but not limited to 75 L, 100 L, 150 L, 300 L, or 500 L). Desirable scalable processes include those wherein there are low to undetectable levels of aggregation as measured by HPSEC or rCGE, typically no more than 5% aggregation by weight of protein down to no more than 0.5% by weight aggregation of protein. Additionally or alternatively, undetectable levels of fragmentation measured in terms of the total peak area representing the intact antigen binding protein may be desired in a scalable process so that at least 80% and as much as 99.5% or higher of the total peak area represents intact antigen binding protein. In other embodiments, the scalable process of the invention produces antigen binding proteins at production efficiency of about from 10 mg/L to about 300 mg/L or higher.

Various techniques have been developed for the production of antibody fragments including proteolytic digestion of intact antibodies and recombinant expression in host cells. With regard to the latter, as described below, Fab, Fv and scFv antibody fragments can all be expressed in and secreted from E. coli, antibody fragments can be isolated from the antibody phage libraries and Fab′-SH fragments can be directly recovered from E. coli and chemically coupled to form F(ab′)2 fragments. In another approach, F(ab′)2 fragments are isolated directly from recombinant host cell culture. In some embodiments, the antigen binding protein is expressed on the surface of a cell, such as a mammalian cell.

In another embodiment there is provided a vector including a nucleic acid described above. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

The antigen binding site may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the antigen binding site-encoding DNA that is inserted into the vector. The signal sequence may be a prokaryotic signal sequence selected, for example, from the group of the alkaline phosphatase, penicillinase, Ipp, or heat-stable enterotoxin II leaders. For yeast secretion the signal sequence may be, e.g., the yeast invertase leader, alpha factor leader, or acid phosphatase leader or the C. albicans glucoamylase leader. In mammalian cell expression, mammalian signal sequences may be used to direct secretion of the protein, such as signal sequences from secreted polypeptides of the same or related species, as well as viral secretory leaders.

Polynucleotide sequences encoding polypeptide components of the antigen binding protein of the invention can be obtained using standard recombinant techniques as described above. Polynucleotides can be synthesized using nucleotide synthesizer or PCR techniques. Once obtained, sequences encoding the polypeptides are inserted into a recombinant vector capable of replicating and expressing heterologous polynucleotides in prokaryotic hosts. Many vectors that are available and known in the art can be used for the purpose of the present invention. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides.

In general, plasmid vectors containing replicon and control sequences which are derived from species compatible with the host cell are used in connection with these hosts. Both expression and cloning vectors contain a nucleic acid sequence that enables the vector to replicate in one or more selected host cells, as well as marking sequences which are capable of providing phenotypic selection in transformed cells. Such sequences are well known for a variety of bacteria, yeast, and viruses. The origin of replication from the plasmid pBR322, which contains genes encoding ampicillin (Amp) and tetracycline (Tet) resistance and thus provides easy means for identifying transformed cells, is suitable for most Gram-negative bacteria, the 2 μm plasmid origin is suitable for yeast, and various viral origins (SV40, polyoma, adenovirus, VSV or BPV) are useful for cloning vectors in mammalian cells. pBR322, its derivatives, or other microbial plasmids or bacteriophage may also contain, or be modified to contain, promoters which can be used by the microbial organism for expression of endogenous proteins.

In addition, phage vectors containing replicon and control sequences that are compatible with the host microorganism can be used as transforming vectors in connection with these hosts. For example, bacteriophage such as λGEM.TM.-11 may be utilized in making a recombinant vector which can be used to transform susceptible host cells such as E. coli LE392.

The expression vectors described herein may comprise two or more promoter-cistron (a cistron being segment of DNA that contains all the information for production of single polypeptide) pairs. A promoter is an untranslated regulatory sequence located upstream (5′) to a cistron that modulates its expression. Prokaryotic promoters typically fall into two classes, inducible and constitutive. Inducible promoter is a promoter that initiates increased levels of transcription of the cistron under its control in response to changes in the culture condition, e.g. the presence or absence of a nutrient or a change in temperature.

A large number of promoters recognized by a variety of potential host cells are well known. The selected promoter can be operably linked to cistron DNA encoding the light or heavy chain by removing the promoter from the source DNA via restriction enzyme digestion and inserting the isolated promoter sequence into the vector of the invention. Both the native promoter sequence and many heterologous promoters may be used to direct amplification and/or expression of the target genes. In some embodiments, heterologous promoters are utilized, as they generally permit greater transcription and higher yields of expressed target gene as compared to the native target polypeptide promoter.

Promoters recognized by a variety of potential host cells are well known.

Promoters suitable for use with prokaryotic hosts include the PhoA promoter, the β-galactamase and lactose promoter systems, alkaline phosphatase, a tryptophan (trp) promoter system and hybrid promoters such as the tac or the trc promoter. Promoters for use in bacterial systems also will contain a Shine-Dalgarno (S.D.) sequence operably linked to the DNA encoding an antigen binding protein of the invention. However, other promoters that are functional in bacteria (such as other known bacterial or phage promoters) are suitable as well. Their nucleotide sequences have been published, thereby enabling a skilled person operably to ligate them to cistrons encoding the target light and heavy chains using linkers or adaptors to supply any required restriction sites.

In one aspect of the invention, each cistron within the recombinant vector comprises a secretion signal sequence component that directs translocation of the expressed polypeptides across a membrane. In general, the signal sequence may be a component of the vector, or it may be a part of the target polypeptide DNA that is inserted into the vector. The signal sequence selected for the purpose of this invention should be one that is recognized and processed (i.e. cleaved by a signal peptidase) by the host cell. For prokaryotic host cells that do not recognize and process the signal sequences native to the heterologous polypeptides, the signal sequence is substituted by a prokaryotic signal sequence selected, for example, from the group consisting of the alkaline phosphatase, penicillinase, Ipp, or heat-stable enterotoxin II (STII) leaders, LamB, PhoE, PelB, OmpA and MBP. In one embodiment of the invention, the signal sequences used in both cistrons of the expression system are STII signal sequences or variants thereof.

In another aspect, the production of the immunoglobulins according to the invention can occur in the cytoplasm of the host cell, and therefore does not require the presence of secretion signal sequences within each cistron. In that regard, immunoglobulin light and heavy chains are expressed, folded and assembled to form functional immunoglobulins within the cytoplasm. Certain host strains (e.g., the E. coli trxB strains) provide cytoplasm conditions that are favourable for disulfide bond formation, thereby permitting proper folding and assembly of expressed protein subunits.

The present invention provides an expression system in which the quantitative ratio of expressed polypeptide components can be modulated in order to maximize the yield of secreted and properly assembled antigen binding proteins of the invention. Such modulation is accomplished at least in part by simultaneously modulating translational strengths for the polypeptide components.

In terms of expression in eukaryotic host cells, the vector components generally include, but are not limited to, one or more of the following: a signal sequence, an origin of replication, one or more marker genes, an enhancer element, a promoter, and a transcription termination sequence.

A vector for use in a eukaryotic host cell may also contain a signal sequence or other polypeptide having a specific cleavage site at the N-terminus of the mature protein or polypeptide of interest. The heterologous signal sequence selected preferably is one that is recognized and processed {i.e., cleaved by a signal peptidase) by the host cell. In mammalian cell expression, mammalian signal sequences as well as viral secretory leaders, for example, the herpes simplex gD signal, are available.

The DNA for such precursor region is ligated in reading frame to DNA encoding the antibody.

Generally, an origin of replication component is not needed for mammalian expression vectors. For example, the SV40 origin may typically be used only because it contains the early promoter.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

An example of suitable selectable markers for mammalian cells are those that enable the identification of cells competent to take up the antigen binding protein-encoding nucleic acid, such as DHFR or thymidine kinase, metallothionein-I and -II, preferably primate metallothionein genes, adenosine deaminase, ornithine decarboxylase, etc. An appropriate host cell when wild-type DHFR is employed is the CHO cell line deficient in DHFR activity (e.g., ATCC CRL-9096), prepared and propagated. For example, cells transformed with the DHFR selection gene are first identified by culturing all of the transformants in a culture medium that contains methotrexate (Mtx), a competitive antagonist of DHFR. Alternatively, host cells (particularly wild-type hosts that contain endogenous DHFR) transformed or co-transformed with DNA sequences encoding an antibody, wild-type DHFR protein, and another selectable marker such as aminoglycoside 3 ‘-phosphotransferase (APH) can be selected by cell growth in medium containing a selection agent for the selectable marker such as an aminoglycosidic antibiotic, e.g., kanamycin, neomycin, or G418.

Expression and cloning vectors usually contain a promoter operably linked to the antigen binding protein encoding nucleic acid sequence to direct mRNA synthesis. Promoters recognized by a variety of potential host cells are well known.

Eukaryotic genes generally have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Examples of suitable promoting sequences for use with yeast hosts include the promoters for 3-phosphoglycerate kinase or other glycolytic enzymes including enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization.

Antigen binding protein transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription of a DNA encoding the antigen binding protein by higher eukaryotes may be increased by inserting an enhancer sequence into the vector. Enhancer sequences include those known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding an antigen binding protein.

In another embodiment there is provided a cell including a vector or nucleic acid described above. The nucleic acid molecule or vector may be present in the genetically modified host cell or host either as an independent molecule outside the genome, preferably as a molecule which is capable of replication, or it may be stably integrated into the genome of the host cell or host.

The host cell of the present invention may be any prokaryotic or eukaryotic cell.

Examples of prokaryotic cells are those generally used for cloning like E. coli or Bacillus subtilis. Furthermore, eukaryotic cells comprise, for example, fungal or animal cells.

Examples for suitable fungal cells are yeast cells, preferably those of the genus Saccharomyces and most preferably those of the species Saccharomyces cerevisiae.

Examples of animal cells are, for instance, insect cells, vertebrate cells, preferably mammalian cells, such as e.g. HEK293, NSO, CHO, MDCK, U2-OS, Hela, NIH3T3, MOLT-4, Jurkat, PC-12, PC-3, IMR, NT2N, Sk-n-sh, CaSki, C33A. These host cells, e.g. CHO-cells, may provide post-translational modifications to the antibody molecules of the invention, including leader peptide removal, folding and assembly of H (heavy) and L (light) chains, glycosylation of the molecule at correct sides and secretion of the functional molecule.

Further suitable cell lines known in the art are obtainable from cell line depositories, like the American Type Culture Collection (ATCC).

In another embodiment there is provided an animal including a cell described above. In certain embodiments, animals and tissues thereof containing a transgene are useful in producing the antigen binding proteins of the invention. The introduction of the nucleic acid molecules as transgenes into non-human hosts and their subsequent expression may be employed for the production of the antigen binding proteins, for example, the expression of such a transgene in the milk of the transgenic animal provide for means of obtaining the antigen binding proteins in quantitative amounts. Useful transgenes in this respect comprise the nucleic acid molecules of the invention, for example, coding sequences for the antigen binding proteins described herein, operatively linked to promoter and/or enhancer structures from a mammary gland specific gene, like casein or beta-lactoglobulin. The animal may be non-human mammals, most preferably mice, rats, sheep, calves, dogs, monkeys or apes.

### Binding to Target Antigen

Methods for determining the successful binding of an antigen binding protein of the invention to its target antigen (ie La) are well known in the art. Non-limiting examples of such methods are described herein in the Examples. Methods for confirmation of the specificity and binding affinity of an antigen binding protein include use of Western blotting, ELISA, immunohistochemistry and Biacore methods, which are all within the skill set of a person skilled in the art.

### Detection Methods

It will be appreciated that the methods for detecting or imaging of the labelled antigen binding proteins for use according to the invention will depend on the nature of the detectable moiety of the protein.

Where the detectable moiety is a radiolabel (radionuclide), the step of detecting is preferably performed using PET, SPECT, or any other suitable method. Optionally, the detection method comprises PET/SPECT, PET/CT imaging or PET/MRI scanning.

The in vivo detection step may be whole body imaging or local imaging at specific sites, such as but not limited to sites of anticipated or likely solid tumour growth.

Following administration (preferably infusion) of the radiolabelled antigen binding protein, it may be practical to wait for a period of time to allow for the agent to accumulate at the site of dying cancer cells presenting La/SSB antigen. Typically, the period of time will be at least about 2 days, at least about 3 days, at least about 4 days, at least about 5 days, at least about 6 days, at least about 7 days, at least about 8 days, at least about 9 days, at least about 10 days. Preferably, the time between administration and detection (eg by PET or other method described here), the period is typically no more than about 10 days, or no more than about 15 days or no more than about 20 days.

In the case where the imaging or detection is using PET, the PET imaging may be performed preferably within 7±2 days of infusion of the radiolabelled antigen binding protein, in particular 5±2 days after the infusion, in order to obtain the optimal imaging results including accumulation of the agent at sites where La/SSB is present.

Imaging will typically be assessed following administration of the radiolabelled antigen binding protein, and detection thereof, by qualitatively assessing the detection of the radiolabelled protein compared with conventional imaging. Quantitative assessment may be on a per lesion basis, including standardized uptake values (SUV) (SUVmax and SUVmean), SUV corrected for lean mass (SUL), metabolic tumor volume (MTV), and Tumour-to-background ratio (TBR).

The Tumor-to-Background Ratio (TBR) will typically be defined as the ratio of the lesion standardized uptake values (SUVmax) over the reference region SUV (liver, blood pool, etc). Comparison between the number, size and other characteristics of lesions detected by PET scan and standard imaging modalities including high-resolution CT/MRI and other potential imaging per patient (depending on the tumor type), type of lesion and indication will be performed.

Imaging Qualitative visual analysis (presence or absence of localized agent uptake associated with tumor, as seen on contrast-enhanced CT, MRI or FDG PET/CT), may be used to evaluate concordance of tumor lesion detection between agent-specific PET/CT and conventional imaging. RECIST 1.1 criteria for conventional imaging may be used as the main tool for concordance comparison vs PET.

In addition to the above, all visible tumoral lesions at conventional imaging may also be compared to PET imaging result.

### Conditions to be Monitored or Treated

It will be appreciated that the antigen binding proteins of the invention have application in treating any number of conditions requiring monitoring for cell death, or requiring treatment to further induce cell death in surrounding tissues (eg to elicit a “the bystander effect”).

In preferred embodiments, the antigen binding proteins of the invention have particular utility in methods involving the monitoring of efficacy of treatment for cancer, or for treating cancer (depending on the form of the antigen binding protein used).

As used herein, methods of treating cancer include methods of inhibiting, preventing or minimising spread or progression of a cancer, including inhibiting or preventing metastasis of cancer.

Examples of cancers which may be monitored or treated according to the methods of the present invention include, pre-neoplastic and neoplastic diseases. Broad examples include breast tumours, colorectal tumours, adenocarcinomas, mesothelioma, bladder tumours, prostate tumours, germ cell tumour, hepatoma/cholongio, carcinoma, neuroendocrine tumours, pituitary neoplasm, small round cell tumour, squamous cell cancer, melanoma, atypical fibroxanthoma, seminomas, nonseminomas, stromal leydig cell tumours, Sertoli cell tumours, skin tumours, kidney tumours, testicular tumours, brain tumours, ovarian tumours, stomach tumours, oral tumours, bladder tumours, bone tumours, cervical tumours, esophageal tumours, laryngeal tumours, liver tumours, lung tumours, vaginal tumours and Wilm's tumour.

Examples of particular cancers include but are not limited to adenocarcinoma, adenoma, adenofibroma, adenolymphoma, adontoma, AIDS related cancers, acoustic neuroma, acute lymphocytic leukaemia, acute myeloid leukaemia, adenocystic carcinoma, adrenocortical cancer, agnogenic myeloid metaplasia, alopecia, alveolar soft-part sarcoma, ameloblastoma, angiokeratoma, angiolymphoid hyperplasia with eosinophilia, angioma sclerosing, angiomatosis, apudoma, anal cancer, angiosarcoma, aplastic anaemia, astrocytoma, ataxia-telangiectasia, basal cell carcinoma (skin), bladder cancer, bone cancers, bowel cancer, brain stem glioma, brain and CNS tumours, breast cancer, branchioma, CNS tumours, carcinoid tumours, cervical cancer, childhood brain tumours, childhood cancer, childhood leukaemia, childhood soft tissue sarcoma, chondrosarcoma, choriocarcinoma, chronic lymphocytic leukaemia, chronic myeloid leukaemia, colorectal cancers, cutaneous T-cell lymphoma, carcinoma (e.g. Walker, basal cell, basosquamous, Brown-Pearce, ductal, Ehrlich tumour, Krebs 2, Merkel cell, mucinous, non-small cell lung, oat cell, papillary, scirrhous, bronchiolar, bronchogenic, squamous cell, and transitional cell), carcinosarcoma, cervical dysplasia, cystosarcoma phyllodies, cementoma, chordoma, choristoma, chondrosarcoma, chondroblastoma, craniopharyngioma, cholangioma, cholesteatoma, cylindroma, cystadenocarcinoma, cystadenoma, dermatofibrosarcoma-protuberans, desmoplastic-small-round-cell-tumour, ductal carcinoma, dysgerminoam, endocrine cancers, endometrial cancer, ependymoma, esophageal cancer, Ewing's sarcoma, extra-hepatic bile duct cancer, eye cancer, eye: melanoma, retinoblastoma, fallopian tube cancer, fanconi anaemia, fibroma, fibrosarcoma, gall bladder cancer, gastric cancer, gastrointestinal cancers, gastrointestinal-carcinoid-tumour, genitourinary cancers, germ cell tumours, gestationaltrophoblastic-disease, glioma, gynaecological cancers, giant cell tumours, ganglioneuroma, glioma, glomangioma, granulosa cell tumour, gynandroblastoma, haematological malignancies, hairy cell leukaemia, head and neck cancer, hepatocellular cancer, hereditary breast cancer, histiocytosis, Hodgkin's disease, human papillomavirus, hydatidiform mole, hypercalcemia, hypopharynx cancer, hamartoma, hemangioendothelioma, hemangioma, hemangiopericytoma, hemangiosarcoma, hemangiosarcoma, histiocytic disorders, histiocytosis malignant, histiocytoma, hepatoma, hidradenoma, hondrosarcoma, immunoproliferative small, opoma, ontraocular melanoma, islet cell cancer, Kaposi's sarcoma, kidney cancer, langerhan's cell-histiocytosis, laryngeal cancer, leiomyosarcoma, leukaemia, li-fraumeni syndrome, lip cancer, liposarcoma, liver cancer, lung cancer, lymphedema, lymphoma, Hodgkin's lymphoma, non-Hodgkin's lymphoma, leigomyosarcoma, leukaemia (e.g. b-cell, mixed cell, null-cell, t-cell, t-cell chronic, htlv-ii-associated, lymphangiosarcoma, lymphocytic acute, lymphocytic chronic, mast-cell and myeloid), leukosarcoma, leydig cell tumour, liposarcoma, leiomyoma, leiomyosarcoma, lymphangioma, lymphangiocytoma, lymphagioma, lymphagiomyoma, lymphangiosarcoma, male breast cancer, malignant-rhabdoid-tumour-of-kidney, medulloblastoma, melanoma, Merkel cell cancer, mesothelioma, metastatic cancer, mouth cancer, multiple endocrine neoplasia, mycosis fungoides, myelodysplastic syndromes, myeloma, myeloproliferative disorders, malignant carcinoid syndrome carcinoid heart disease, medulloblastoma, meningioma, melanoma, mesenchymoma, mesonephroma, mesothelioma, myoblastoma, myoma, myosarcoma, myxoma, myxosarcoma, nasal cancer, nasopharyngeal cancer, nephroblastoma, neuroblastoma, neurofibromatosis, Nijmegen breakage syndrome, non-melanoma skin cancer, non-small-cell-lung-cancer-(nsclc), neurilemmoma, neuroblastoma, neuroepithelioma, neurofibromatosis, neurofibroma, neuroma, neoplasms (e.g. bone, breast, digestive system, colorectal, liver), ocular cancers, oesophageal cancer, oral cavity cancer, oropharynx cancer, osteosarcoma, ostomy ovarian cancer, pancreas cancer, paranasal cancer, parathyroid cancer, parotid gland cancer, penile cancer, peripheral-neuroectodermal-tumours, pituitary cancer, polycythemia vera, prostate cancer, osteoma, osteosarcoma, ovarian carcinoma, papilloma, paraganglioma, paraganglioma nonchromaffin, pinealoma, plasmacytoma, protooncogene, rare-cancers-and-associated-disorders, renal cell carcinoma, retinoblastoma, rhabdomyosarcoma, Rothmund-Thomson syndrome, reticuloendotheliosis, rhabdomyoma, salivary gland cancer, sarcoma, schwannoma, Sezary syndrome, skin cancer, small cell lung cancer (sclc), small intestine cancer, soft tissue sarcoma, spinal cord tumours, squamous-cell-carcinoma-(skin), stomach cancer, synovial sarcoma, sarcoma (e.g. Ewing's experimental, Kaposi's and mast-cell sarcomas), Sertoli cell tumour, synovioma, testicular cancer, thymus cancer, thyroid cancer, transitional-cell-cancer-(bladder), transitional-cell-cancer-(renal-pelvis-/-ureter), trophoblastic cancer, teratoma, theca cell tumour, thymoma, trophoblastic tumour, urethral cancer, urinary system cancer, uroplakins, uterine sarcoma, uterus cancer, vaginal cancer, vulva cancer, Waldenstrom's-macroglobulinemia and Wilms' tumour.

Other diseases and conditions include various inflammatory conditions. Examples may include a proliferative component. Particular examples include acne, angina, arthritis, aspiration pneumonia, disease, empyema, gastroenteritis, inflammation, intestinal flu, nee, necrotising enterocolitis, pelvic inflammatory disease, pharyngitis, pid, pleurisy, raw throat, redness, rubor, sore throat, stomach flu and urinary tract infections, chronic inflammatory demyelinating polyneuropathy, chronic inflammatory demyelinating polyradiculoneuropathy, chronic inflammatory demyelinating polyneuropathy or chronic inflammatory demyelinating polyradiculoneuropathy.

### Monitoring Response

The methods of the present invention find utility in monitoring the response of a subject to a treatment for cancer. The present invention includes monitoring the efficacy of a treatment with a cytotoxic therapy, wherein the treatment includes but is not limited to administration of any one or more of: chemotherapy, chemo-immunotherapy, immunotherapy, autologous stem cell transfer (ASCT), external beam radiation, stereotactic ablative body radiotherapy, immuno-radiation or combinations thereof.

Typically the methods for monitoring response to treatment include a comparison of an amount of La/SSB antigen (eg as detected by the extent of binding by an antigen binding protein of the invention) after or during treatment compared to the amount detectable prior to treatment or at an earlier time point during treatment. In accordance with the methods of the present invention, this typically involves comparing the amount of radiolabelled antigen binding protein detected in the patient or cancer at different time points although the comparison may also be in relation to a reference data set comprising data from one or more individuals.

In certain embodiments, the detecting may result in data which are used to compile training data for development of a deep learning algorithm for enabling artificial-intelligence based self-learning for assessing the efficacy of treatment. This includes the use of such Al-based methods for compiling data from the individual patient over the course of their treatment.

Further still the methods may involve using deep learning models or algorithms to determine the likelihood of success of a cytotoxic therapy. In certain examples, the training data (or reference data set upon which the model or algorithm is based), may comprise data from one or more individuals who previously responded to treatment with a cytotoxic therapy. In further examples, the training data (or reference data upon which the deep learning model or algorithm is based) may comprises data from one or more individuals who do not have cancer, or who did not respond to treatment with a cytotoxic therapy. The training data may comprise data from a combination of patients who had different responses to treatment with a cytotoxic therapy.

In certain embodiments, the detecting data obtained from the methods described herein may be assessed relative to a disease classification model that has been generated using machine learning techniques using training data (or reference data set upon which the deep learning algorithm is based), comprising data from one or more individuals who previously responded to treatment with a cytotoxic therapy and/or from one or more individuals who did not respond to treatment with a cytotoxic therapy.

Accordingly, Al-based approaches described herein can serve as a platform on which a variety of tools and techniques for detecting, evaluating, and predicting cancer status for patients can be built. Moreover, since the Al-based tools as described herein can identify a variety of important tissue regions across an entire body of a patient, and then transfer those identifications (e.g., segmentation maps) from anatomical images to various functional images, they can be used as a building block for analysis techniques based on a variety of different imaging methods that obtain contrast via a variety of different radiopharmaceuticals.

The use of Al-based technologies using machine learning techniques (such as Convolution Neural Networds) and the like to monitor disease progression or response to treatment, based on the imaging techniques described herein, are generally known in the art, and are described for example in WO2020144134 and U.S. Pat. No. 11,443,201, incorporated herein by reference.

As used herein, a level or amount that is higher than the reference dataset or amount generally refers to an amount that is at least more than about 10%, at least about 20%, at least about 30%, at least about 40%, at least about 50%, at least about 60%, at least about 70%, at least about 80%, at least about 90% or at least 100% or greater.

As used herein, a level or amount that is lower than the reference dataset or amount generally refers to an amount that is at least about 10%, at least about 20%, at least about 30%, at least about 40%, at least about 50%, at least about 60%, at least about 70%, at least about 80%, at least about 90% or at least 100% or more, lower than the amount in the reference dataset.

The same, higher or lower amount of level or amount generally refers to an amount that differs by no more than about 5% preferably no more than 10% from the reference dataset.

The skilled person will be familiar with methods for determining statically significant differences between the level or amount of detectable radiolabelled antigen binding protein for the purposes of making the comparison required for the present invention.

In accordance with the present specification, determining the response to treatment of a cancer includes stabilisation of the disease or the slowing down or cessation of disease progression. ‘Response to treatment’ refers to therapeutic treatment wherein the object is to slow down (lessen) an undesired physiological change or disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. Treatment can also mean prolonging survival as compared to expected survival if not receiving treatment. Treatment may not necessarily result in the complete clearance of a disease or disorder but may reduce or minimise complications and side effects of infection and the progression of a disease or disorder.

As used herein, a positive response of an individual to a treatment includes an increase in the progression free survival of the individual. Alternatively, a positive response of an individual to a treatment includes an increase in the overall survival of the individual. Accordingly, the present invention also finds utility in predicting the overall survival or progression free survival of an individual receiving/requiring treatment for a the cancer.

As used herein “overall survival” (OS) refers to the length of time from either the date of diagnosis or the start of treatment for a cancer, that patients diagnosed with the cancer are still alive. In a clinical trial, measuring the overall survival is one way to see how well a new treatment works.

‘Overall survival’ or ‘OS’ is well known to one of skill in the art and refers to the fate of the patient after an event, preferably after the start or end of a treatment regime, despite the possibility that the cause of death in a patient is not directly due to the effects of the disease (cancer). In other words, it refers to the prognosis that the patient will not die because of cancer, preferably within at least 1 year, at least 2 years, at least 3 years, at least 4 years, at least 5 years, at least 10 years or at least 15 years.

As used herein “progression free survival” (PFS) refers to the length of time during and after the treatment of a disease, such as cancer, that a patient lives with the disease but it does not get worse. In a clinical trial, measuring the progression-free survival is one way to see how well a new treatment works.

‘Responding to a treatment regime’ refers to a clinically or biochemically favourable detectable response to a treatment. Typically, a favourable response is survival measured at a later time point after treatment, for example, 1, 2, 3, or 4 years post treatment.

### Compositions

In some examples, an antigen binding protein as described herein can be administered orally, parenterally, by inhalation spray, adsorption, absorption, topically, rectally, nasally, bucally, vaginally, intraventricularly, via an implanted reservoir in dosage formulations containing conventional non-toxic pharmaceutically acceptable carriers, or by any other convenient dosage form. The term “parenteral” as used herein includes subcutaneous, intravenous, intramuscular, intraperitoneal, intrathecal, intraventricular, intrasternal, and intracranial injection or infusion techniques.

Methods for preparing an antigen binding protein into a suitable form for administration to a subject (e.g. a pharmaceutical composition) are known in the art and include, for example, methods as described in Remington's Pharmaceutical Sciences (18th ed., Mack Publishing Co., Easton, Pa., 1990) and U.S. Pharmacopeia: National Formulary (Mack Publishing Company, Easton, Pa., 1984). In some embodiments, the antigen binding protein may be administered via administration of a nucleic acid or vector encoding the protein, for expression in cells in a subject.

The pharmaceutical compositions of this invention are particularly useful for parenteral administration, such as intravenous administration or administration into a body cavity or lumen of an organ or joint. The compositions for administration will commonly comprise a solution of an antigen binding protein dissolved in a pharmaceutically acceptable carrier, for example an aqueous carrier. A variety of aqueous carriers can be used, e.g., buffered saline and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of an antigen binding protein of the present invention in these formulations can vary widely, and will be selected primarily based on fluid volumes, viscosities, body weight and the like in accordance with the particular mode of administration selected and the patient's needs. Exemplary carriers include water, saline, Ringer's solution, dextrose solution, and 5% human serum albumin. Non-aqueous vehicles such as mixed oils and ethyl oleate may also be used. Liposomes may also be used as carriers. The vehicles may contain minor amounts of additives that enhance isotonicity and chemical stability, e.g., buffers and preservatives.

The antigen binding proteins of the present invention may be formulated for local or topical administration, such as for topical application to the skin or tissue requiring treatment. Formulations for topical administration typically comprise a topical vehicle combined with active agent(s), with or without additional optional components. The pharmaceutical compositions of the invention may be in the form of a spray, cream, gel, lotion or the like for topical administration.

Suitable topical vehicles and additional components are well known in the art, and it will be apparent that the choice of a vehicle will depend on the particular physical form and mode of delivery. Topical vehicles include organic solvents such as alcohols (for example, ethanol, iso-propyl alcohol or glycerine), glycols such as butylene, isoprene or propylene glycol, aliphatic alcohols such as lanolin, mixtures of water and organic solvents and mixtures of organic solvents such as alcohol and glycerine, lipid-based materials such as fatty acids, acylglycerols including oils such as mineral oil, and fats of natural or synthetic origin, phosphoglycerides, sphingolipids and waxes, protein-based materials such as collagen and gelatine, silicone-based materials (both nonvolatile and volatile), and hydrocarbon-based materials such as microsponges and polymer matrices.

A composition may further include one or more components adapted to improve the stability or effectiveness of the applied formulation, such as stabilising agents, suspending agents, emulsifying agents, viscosity adjusters, gelling agents, preservatives, antioxidants, skin penetration enhancers, moisturisers and sustained release materials. Examples of such components are described in Martindale—The Extra Pharmacopoeia (Pharmaceutical Press, London 1993) and Martin (ed.), Remington's Pharmaceutical Sciences. Formulations may comprise microcapsules, such as hydroxymethylcellulose or gelatine-microcapsules, liposomes, albumin microspheres, microemulsions, nanoparticles or nanocapsules.

A topical formulation may be prepared in a variety of physical forms including, for example, solids, pastes, creams, foams, lotions, gels, powders, aqueous liquids, emulsions, sprays and skin patches. The physical appearance and viscosity of such forms can be governed by the presence and amount of emulsifier(s) and viscosity adjuster(s) present in the formulation. Solids are generally firm and non-pourable and commonly are formulated as bars or sticks, or in particulate form. Solids can be opaque or transparent, and optionally can contain solvents, emulsifiers, moisturisers, emollients, fragrances, dyes/colorants, preservatives and other active ingredients that increase or enhance the efficacy of the final product. Creams and lotions are often similar to one another, differing mainly in their viscosity. Both lotions and creams may be opaque, translucent or clear and often contain emulsifiers, solvents, and viscosity adjusting agents, as well as moisturisers, emollients, fragrances, dyes/colorants, preservatives and other active ingredients that increase or enhance the efficacy of the final product.

Gels can be prepared with a range of viscosities, from thick or high viscosity to thin or low viscosity. These formulations, like those of lotions and creams, may also contain solvents, emulsifiers, moisturisers, emollients, fragrances, dyes/colorants, preservatives and other active ingredients that increase or enhance the efficacy of the final product. Liquids are thinner than creams, lotions, or gels, and often do not contain emulsifiers. Liquid topical products often contain solvents, emulsifiers, moisturisers, emollients, fragrances, dyes/colorants, preservatives and other active ingredients that increase or enhance the efficacy of the final product.

Emulsifiers for use in topical formulations include, but are not limited to, ionic emulsifiers, cetearyl alcohol, non-ionic emulsifiers like polyoxyethylene oleyl ether, PEG-40 stearate, ceteareth-12, ceteareth-20, ceteareth-30, ceteareth alcohol, PEG-100 stearate and glyceryl stearate. Suitable viscosity adjusting agents include, but are not limited to, protective colloids or nonionic gums such as hydroxyethylcellulose, xanthan gum, magnesium aluminium silicate, silica, microcrystalline wax, beeswax, paraffin, and cetyl palmitate. A gel composition may be formed by the addition of a gelling agent such as chitosan, methyl cellulose, ethyl cellulose, polyvinyl alcohol, polyquaterniums, hydroxyethylcellulose, hydroxypropylcellulose, hydroxypropylmethylcellulose, carbomer or ammoniated glycyrrhizinate. Suitable surfactants include, but are not limited to, nonionic, amphoteric, ionic and anionic surfactants. For example, one or more of dimethicone copolyol, polysorbate 20, polysorbate 40, polysorbate 60, polysorbate 80, lauramide DEA, cocamide DEA, and cocamide MEA, oleyl betaine, cocamidopropyl phosphatidyl PG-dimonium chloride, and ammonium laureth sulfate may be used within topical formulations.

Preservatives include, but are not limited to, antimicrobials such as methylparaben, propylparaben, sorbic acid, benzoic acid, and formaldehyde, as well as physical stabilisers and antioxidants such as vitamin E, sodium ascorbate/ascorbic acid and propyl gallate. Suitable moisturisers include, but are not limited to, lactic acid and other hydroxy acids and their salts, glycerine, propylene glycol, and butylene glycol. Suitable emollients include lanolin alcohol, lanolin, lanolin derivatives, cholesterol, petrolatum, isostearyl neopentanoate and mineral oils. Suitable fragrances and colours include, but are not limited to, FD&C Red No. 40 and FD&C Yellow No. 5. Other suitable additional ingredients that may be included in a topical formulation include, but are not limited to, abrasives, absorbents, anticaking agents, antifoaming agents, antistatic agents, astringents (such as witch hazel), alcohol and herbal extracts such as chamomile extract, binders/excipients, buffering agents, chelating agents, film forming agents, conditioning agents, propellants, opacifying agents, pH adjusters and protectants.

Typical modes of delivery for topical compositions include application using the fingers, application using a physical applicator such as a cloth, tissue, swab, stick or brush, spraying including mist, aerosol or foam spraying, dropper application, sprinkling, soaking, and rinsing. Controlled release vehicles can also be used, and compositions may be formulated for transdermal administration (for example, as a transdermal patch).

Upon formulation, an antigen binding protein of the present invention will be administered in a manner compatible with the dosage formulation and in such amount as is therapeutically/prophylactically effective. Formulations are easily administered in a variety of dosage forms, such as the type of injectable solutions described above, but other pharmaceutically acceptable forms are also contemplated, e.g., tablets, pills, capsules or other solids for oral administration, suppositories, pessaries, nasal solutions or sprays, aerosols, inhalants, liposomal forms and the like. Pharmaceutical “slow release” capsules or compositions may also be used. Slow release formulations are generally designed to give a constant drug level over an extended period and may be used to deliver an antigen binding protein of the present invention. In some embodiments, a composition is directly administered to a site, such as a tumor.

WO2002/080967 describes compositions and methods for administering aerosolized compositions comprising antibodies for the treatment of, e.g., asthma, which are also suitable for administration of an antigen binding protein of the present invention.

### Dosage and Timing of Administration

Suitable dosages of an antigen binding protein of the present invention will vary depending on the specific an antigen binding protein, the condition to be treated and/or the subject being treated. It is within the ability of a skilled physician to determine a suitable dosage, e.g., by commencing with a sub-optimal dosage and incrementally modifying the dosage to determine an optimal or useful dosage. Alternatively, to determine an appropriate dosage for treatment/prophylaxis, data from the cell culture assays or animal studies are used, wherein a suitable dose is within a range of circulating concentrations that include the ED₅₀ of the active compound with little or no toxicity. The dosage may vary within this range depending upon the dosage form employed and the route of administration utilized. A therapeutically/prophylactically effective dose can be estimated initially from cell culture assays. A dose may be formulated in animal models to achieve a circulating plasma concentration range that includes the IC₅₀ (i.e., the concentration or amount of the compound which achieves a half-maximal inhibition of symptoms) as determined in cell culture. Such information can be used to more accurately determine useful doses in humans.

In some examples, a method of the present invention comprises administering a prophylactically or therapeutically effective amount of a protein described herein.

The phrase “therapeutically effective amount” or “effective amount”, generally refers to an amount of an antigen binding protein of the invention that (i) treats the particular disease, condition, or disorder, (ii) attenuates, ameliorates, or eliminates one or more symptoms of the particular disease, condition, or disorder, or (iii) delays the onset of one or more symptoms of the particular disease, condition, or disorder described herein. Undesirable effects, e.g. side effects, are sometimes manifested along with the desired therapeutic effect; hence, a practitioner balances the potential benefits against the potential risks in determining what is an appropriate “effective amount”.

A therapeutically effective amount of a treatment may also include a “preventative” or “prophylactically effective amount,” which is any amount of the treatment administered to a subject at risk of developing a cancer (eg a subject having a pre-malignant condition) or of suffering a recurrence of cancer, that inhibits the development or recurrence of the cancer. In certain embodiments, the prophylactically effective amount prevents the development or recurrence of the cancer entirely. “Inhibiting” or “preventing” the development or recurrence of a cancer means either lessening the likelihood of the cancer's development or recurrence, or preventing the development or recurrence of the cancer entirely.

The amount to be administered to a subject will depend on the particular characteristics of the condition to be treated, the type and stage of condition being treated, the mode of administration, and the characteristics of the subject, such as general health, other diseases, age, sex, genotype, and body weight. A person skilled in the art will be able to determine appropriate dosages depending on these and other factors. Accordingly, this term is not to be construed to limit the present invention to a specific quantity, e.g., weight or amount of protein(s), rather the present invention encompasses any amount of the antigen binding protein(s) sufficient to achieve the stated result in a subject.

The terms “treatment” or “treating” of a subject includes the application or administration of a compound of the invention to a subject with the purpose of delaying, slowing, stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” refers to any indication of success in the treatment or amelioration of an injury, pathology or condition, including any objective or subjective parameter such as abatement; remission; lessening of the rate of worsening; lessening severity of the disease; stabilization, diminishing of symptoms or making the injury, pathology or condition more tolerable to the subject; slowing in the rate of degeneration or decline; making the final point of degeneration less debilitating; or improving a subject's physical or mental well-being.

As used herein, minimising or preventing the progression of cancer means treating the subject so as to prevent or delay the recurrence or metastasis of a tumor, or to prevent growth of an existing tumor. Minimising or preventing the progression of cancer includes preventing or delaying the recurrence of cancer, or preventing growth of an existing tumor, following treatment of cancer. The recurrence that is being prevented includes a recurrence for example, in the tumor bed, following surgical excision. Alternatively, recurrence includes metastasis of the cancer in another part of the body. The terms “preventing recurrence” and “preventing relapse” as used herein, are interchangeable.

The present invention also includes methods of preventing the development of cancer in an individual. For example, the individual for whom prevention of cancer is required may be considered to be at risk of developing cancer, but does not yet have detectable cancer. An individual at risk of the development of cancer may be an individual with a family history of cancer, and/or an individual for whom genetic testing or other testing indicates a high risk or high likelihood of the development of cancer. The individual may have cancer stem cells but does not yet have any detectable tumors. It will be understood that methods of preventing the development of cancer include methods of delaying the onset of cancer in a subject.

The terms “subject” and “patient” will be understood to be interchangeable. Although the invention finds application in humans, the invention is also useful for therapeutic veterinary purposes. The invention is useful for domestic or farm animals such as cattle, sheep, horses and poultry; for companion animals such as cats and dogs; and for zoo animals.

In accordance with some embodiments of methods of the invention, an antigen binding protein of the invention may be administered in combination with a first cytotoxic therapy. The administration may be simultaneous, consecutive, separate or sequential. Alternatively, administration may be staggered. The first and second cytotoxic therapies may also be administered at the same frequency or at different frequencies.

For example, the first cytotoxic therapy (such as chemotherapy, EBRT, SABR, immunotherapy, radio-immunotherapy) may be administered at the same time as the second cytotoxic therapy (in the form of an antigen binding protein of the invention, optionally an antigen binding protein of the invention conjugated to a radionuclide). Alternatively, the first cytotoxic therapy (such as chemotherapy, EBRT, SABR, immunotherapy, radio-immunotherapy) may be administered at a first time point, and the second cytotoxic therapy (in the form of an antigen binding protein of the invention, optionally an antigen binding protein of the invention conjugated to a radionuclide), may be administered at a second time point which is after administration of the first therapy. The period of time between administration of the first and second cytotoxic therapies may be less than 24 hours, or may be at least 24 hours, at least 48 hours, at least 3 days, at least 4 days, at least 5 days, at least 6 days, at least 7 days, at least 10 days, at least 14 days or more. Preferably the time between administration of the first and second cytotoxic therapies is no more than 14 days, no more than 21 days or no more than 28 days.

In certain embodiments the treatment may comprise one or more treatment cycles, wherein each cycle comprises administration of a first cytotoxic therapy (such as chemotherapy, EBRT, SABR, immunotherapy, radio-immunotherapy) and a second cytotoxic therapy (in the form of an antigen binding protein of the invention, optionally an antigen binding protein of the invention conjugated to a radionuclide).

### Kits

In another embodiment there is provided a kit or article of manufacture including an antigen binding protein, immunoglobulin variable domain, antibody, dab, scFv, Fab, Fab′, F(ab′)2, Fv fragment, diabody, triabody, linear antibody, single-chain antibody molecule, or multispecific antibody, fusion protein, conjugate or pharmaceutical composition as described above, or a nucleic acid or vector.

In other embodiments there is provided a kit for use in a use mentioned above, the kit including:
a container holding a therapeutic composition in the form of one or more of an antigen binding protein, immunoglobulin variable domain, antibody, dab, scFv, Fab, Fab′, F(ab′)2, Fv fragment, diabody, triabody, linear antibody, single-chain antibody molecule, or multispecific antibody, fusion protein, conjugate or pharmaceutical composition;
a label or package insert with instructions for use.

The kit or “article of manufacture” may comprise a container and a label or package insert on or associated with the container. Suitable containers include, for example, bottles, vials, syringes, blister pack, etc. The containers may be formed from a variety of materials such as glass or plastic. The container holds a therapeutic composition which is effective for treating the condition and may have a sterile access port (for example the container may be an intravenous solution bag or a vial having a stopper pierceable by a hypodermic injection needle). The label or package insert indicates that the therapeutic composition is used for treating the condition of choice. In one embodiment, the label or package insert includes instructions for use.

The kit may comprise (a) a therapeutic composition; and (b) a second container with a second active principle or ingredient contained therein. The kit in this embodiment of the invention may further comprise a package insert indicating that the and other active principle can be used to treat a disorder or prevent a complication stemming from cancer. Alternatively, or additionally, the kit may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In certain embodiments the therapeutic composition may be provided in the form of a device, disposable or reusable, including a receptacle for holding the therapeutic composition. In one embodiment, the device is a syringe. The device may hold 1-2 mL of the therapeutic composition. The therapeutic composition may be provided in the device in a state that is ready for use or in a state requiring mixing or addition of further components.

In other embodiments there is provided a kit for use in a diagnostic application mentioned above, the kit including:
a container holding a diagnostic composition in the form of one or more of an antigen binding protein, immunoglobulin variable domain, antibody, Fab, dab, scFv, diabody, triabody, fusion protein or conjugate;
a label or package insert with instructions for use.

The kit may comprise (a) a diagnostic composition; and (b) a second container with a second diagnostic agent or second label contained therein. It may further include other materials desirable from a commercial and user standpoint, including other buffers, diluents, filters etc.

It will be understood that the invention disclosed and defined in this specification extends to all alternative combinations of two or more of the individual features mentioned or evident from the text or drawings. All of these different combinations constitute various alternative aspects of the invention.

The examples that follow are intended to illustrate but in no way limit the present invention.

### EXAMPLES

### Example 1: Humanisation of APOMAB

A parental, chimeric anti-La antibody (APOMAB) was obtained.

The inventors sought to generate humanised antigen binding proteins for binding to La/SSB antigen that balanced several benchmarked measures including antibody immunogenicity risk, and performance and developability characteristics. The inventors needed to consider a large number of heavy chain and light chain combinations.

Potential manufacturing liabilities were identified in the VH and Vκ sequences of the parental chimeric antibody. Consequently, three further heavy chain variants and two light chain variants were designed, and the variants were synthesised. Combinations of heavy and light chain variants were transfected to CHO cells and proteins obtained from supernatants.

For the large number of different heavy chain and light chain combinations, the inventors assessed a number of parameters and narrowed down the potential leads to 8 heavy chain and 8 light chain humanised sequence variants. These were screened in silico for predictive immunogenicity and manufacturability assessment, and in vivo, for their ability to retain binding to La protein. Using the single cycle kinetic (SCK) assay format by surface plasmon resonance (SPR), binding kinetic parameters such as Kₐ, K_{d}, and K_{D} were determined for the humanised variants. The antigen used for the SPR binding studies was the La/SSB N-terminal domain (La-NTD) recombinant protein.

Two humanised heavy chains (VH3 and VH6) and two humanised light chains (VK6 and VK8) were selected for further development and screening of the following VH/VL combinations:

| Proposed antibodies | | VH name | VK name |
|---|---|---|---|
| | Chimeric parental | chAPOMAB_VH0 | chAPOMAB_VK0 |
| 1 | Humanized-v1 | chAPOMAB_VH0 | VK6 |
| 2 | Humanized-v2 | chAPOMAB_VH0 | VK8 |
| 3 | Humanized-v3 | VH3 | chAPOMAB_VK0 |
| 4 | Humanized-v4 | VH3 | VK6 |
| 5 | Humanized-v5 | VH3 | VK8 |
| 6 | Humanized-v6 | VH6 | chAPOMAB_VK0 |
| 7 | Humanized-v7 | VH6 | VK6 |
| 8 | Humanized-v8 | VH6 | VK8 |

Sequences of each of the VH and VK chains are provided in Table 2.

Variants of the humanised antibodies comprising substitutions for reducing C1q binding (to render the antibodies CDC-deficient) and reducing FcRN binding (to reduce serum half-life), were also prepared. These antibodies comprise the substitutions (K322A and/or H310A and H435Q) and are also referred to herein as “K322A” or “K322A_RadMab”).

| | Quantity | Conc. | Volume | Monomer |
|---|---|---|---|---|
| | (mg) | (mg/ml) | (ml) | (%) |
| chAPOMAB_VH0/VK0_K322A | 106 | 4.25 | 25 | 98.7% |
| VH3/VK6_K322A | 57 | 2.38 | 24 | 95.0% |
| VH6/VK8_K322A | 54 | 2.92 | 18.5 | 96.2% |
| VH3/VK6_K322_RadMab | 6 | 0.23 | 28 | 79.5% |
| VH6/VK8_K322A_RadMab | 48 | 1.10 | 43.5 | 100.0% |

### Example 2: Characterisation of Humanised Antibodies

All humanised antibodies were assessed for their ability to bind to target (La protein).

More specifically, antibodies were assessed for their ability to bind to La N-terminal domain using Biacore binding analysis. The results are summarised in the below table:

| | | | | R_{MAX} | Chi² |
|---|---|---|---|---|---|
| | Kₐ (1/Ms) | K_{d} (1/s) | K_{D} (M) | (RU) | (RU²) |
| chAPOMAB_VH)/VKO_K322A | 2.75E+04 | 5.02E−04 | 1.82E−08 | 37.4 | 0.0732 |
| VH3/VK6_K322A | 5.69E+04 | 7.76E−04 | 1.36E−08 | 41.3 | 0.152 |
| VH6/VK8_K322A | 5.37E+04 | 7.77E−04 | 1.45E−08 | 29.8 | 0.0874 |
| VH3/VK6_K322_RadMab | 4.34E+04 | 3.58E−04 | 8.27E−09 | 42.1 | 0.05 |
| VH6/VK8_K322A_RadMab | 4.10E+04 | 3.57E−04 | 8.72E−09 | 40 | 0.113 |

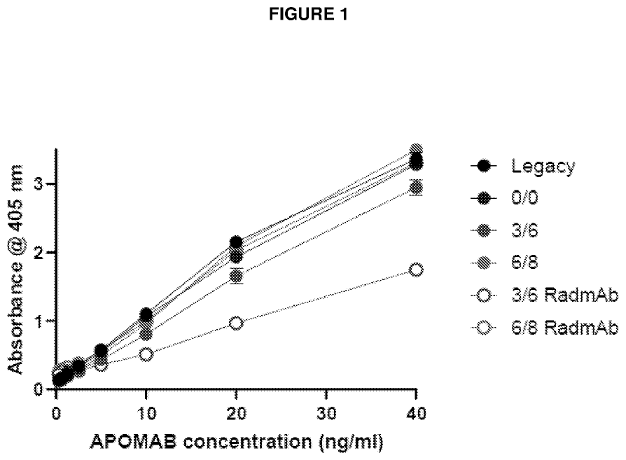

An ELISA assay for binding to La N terminal domain was also performed. The results, shown in FIG. 1, also indicate that all antibodies bind with similar affinity to La N-terminal domain, although binding affinity was slightly lower for VH3/VK6_K322A_RadMab.

### Example 3: Binding of Antibodies to Human Lung Cancer Cells

Antibodies were assessed using flow cytometry analysis, for their ability to bind to human lung cancer cells (H460).

H460 cells were split into the following groups:
untreated
treated with 20 μg/ml cisplatin for 72 h (to induce cell death)
untreated and permeabilized with formalin/methanol to expose all intracellular La/SSB.

Cells were collected, 10⁵ cells were then stained with 5 μg/ml humanised antibody (hAPOMAB) (or rituximab as negative control) in triplicate, followed by staining with anti-human Alexa Fluor 647 secondary antibody and propidium iodide (PI). The percentage of APOMAB positive cells and the median fluorescence intensity (MFI) was determined for each humanised antibody.

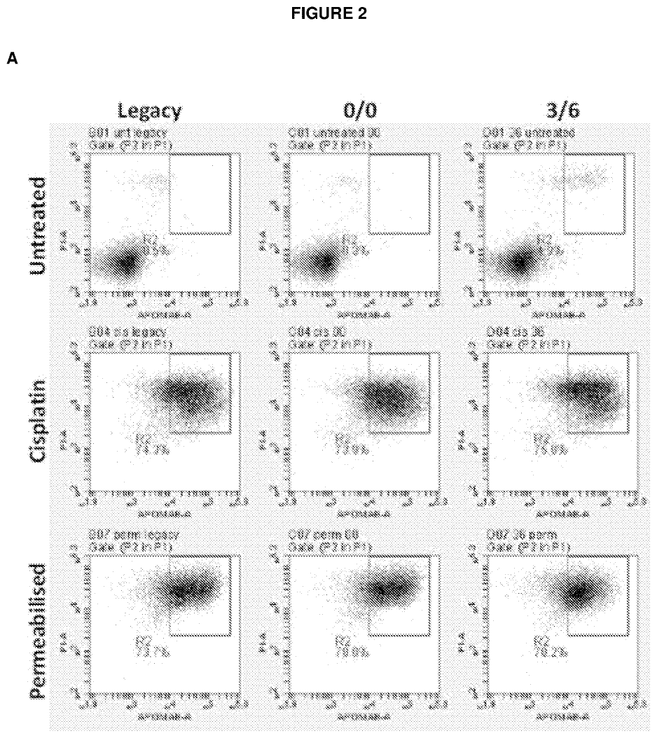

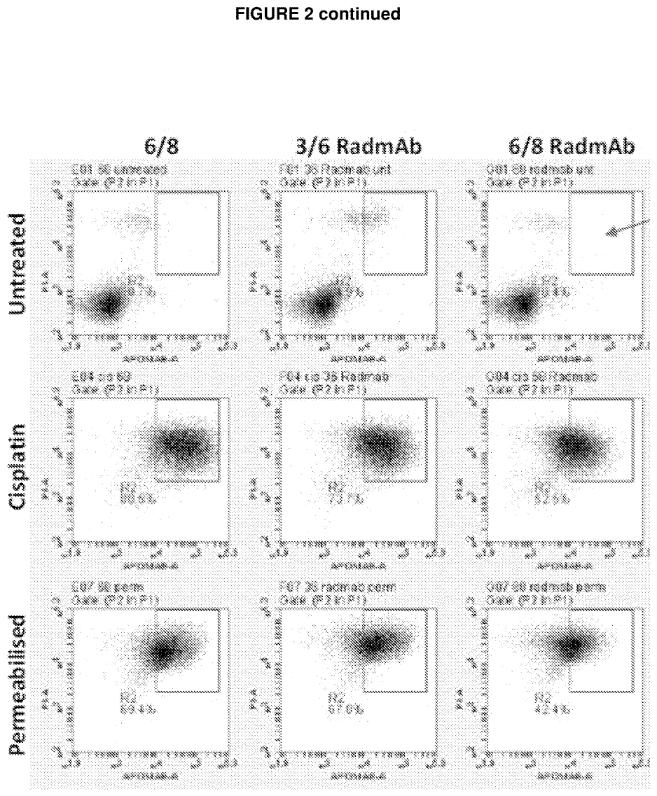

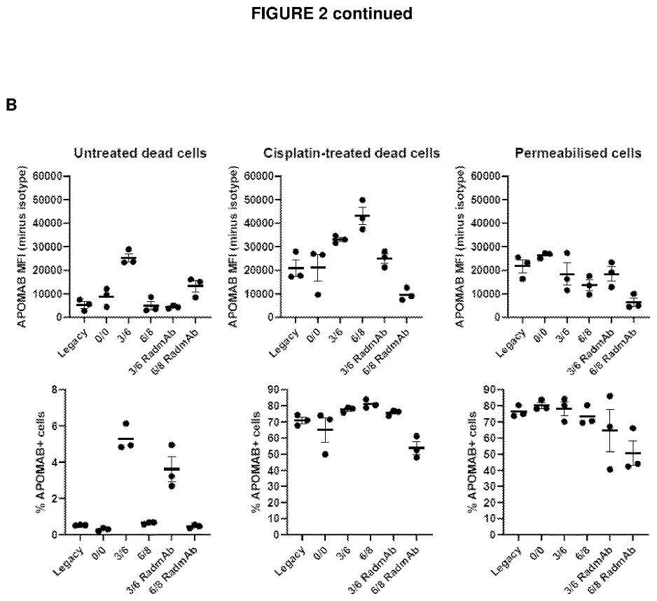

The results of the flow cytometry are shown in FIG. 2. Humanised antibodies bound only to PI-positive dead or permealised cancer cells, and did not bind to live cells.

The parental (legacy) chimeric APOMAB and 0/0 antibody bound with similar affinities to each other.

Humanised antibodies VH3/VK6 and VH3/VK6_K322_RadMab bound more to dead cells in untreated cultures.

Antibodies VH3/VK6 and VH6/VK8 showed equivalent/higher binding (MFI) to cisplatin-treated dead cells compared to the parental chimeric antibody (VH0/VK0).

### Example 4: DFO Conjugation of Humanised Antibodies

Approximately 7 mg of each of the choAPOMAB_VH0/choAPOMAB_VK0, VH3/VK6, VH6/VK8 and VH6/VK8_K322A_RadMab antibodies were conjugated with DFO-Sq.

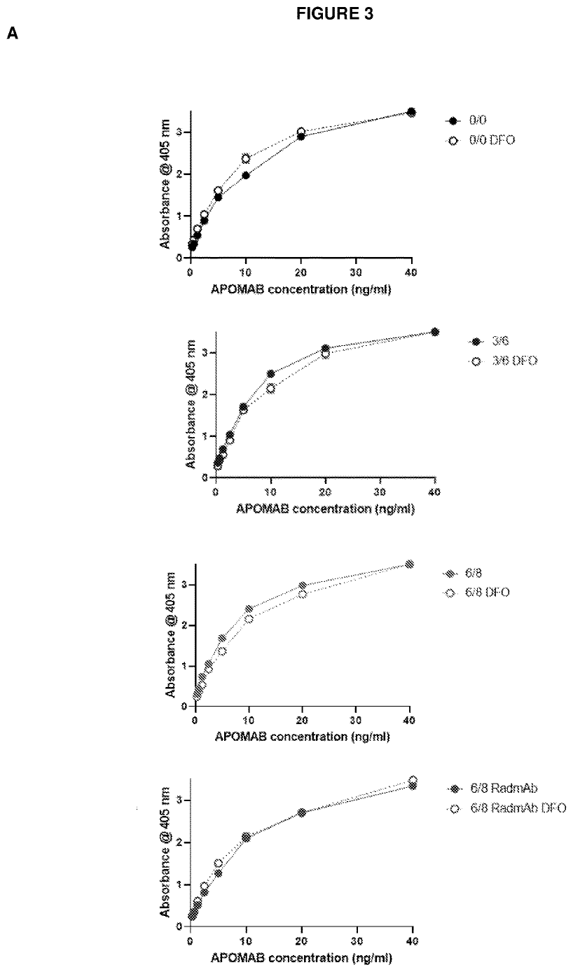

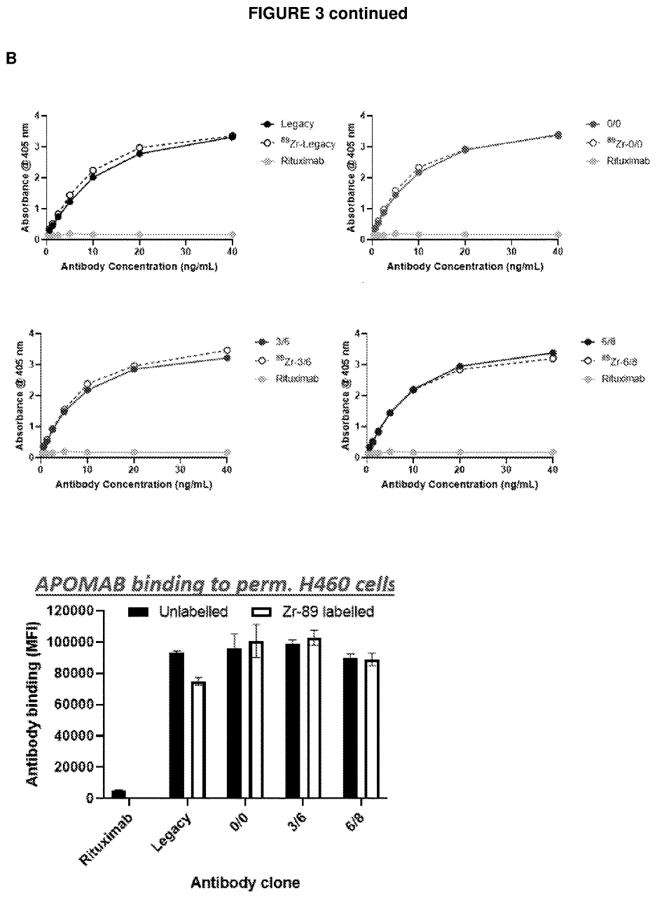

The binding affinity of the DFO-conjugated and unconjugated antibodies was assessed by ELISA binding to La N-terminal domain. The binding affinities of the conjugated and unconjugated antibodies were found to be very similar, indicating that conjugation did not impact binding affinity, as shown in FIG. 3A.

Following DFO conjugation, antibodies were subsequently labelled with Zirconium-89 radioisotope. The biding affinities of radiolabelled antibodies (confirmed by ELISA and flow cytometry) showed no significant differences in binding compared to DFO-conjugated (unlabelled) antibody), as shown in FIG. 3B.

### Example 5: In Vivo Imaging of ⁸⁹Zr Labelled Humanised Antibodies

Mice bearing a H460 lung cancer xenograft were obtained according to the method previously published in Liapis et al., (2021), Mol. Imaging. Biol. 6:914-928.

Mice with established tumours were treated with cisplatin, followed the next day with 5 MBq ⁸⁹Zr-APOMAB and were subjected to PET imaging daily for 3 days. Tissues were collected on the final day for analysis of biodistribution.

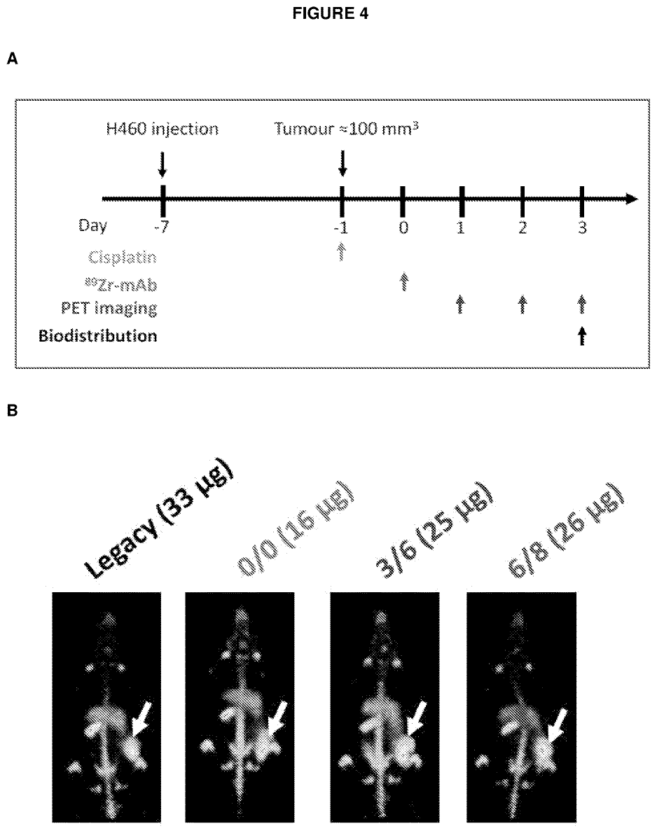

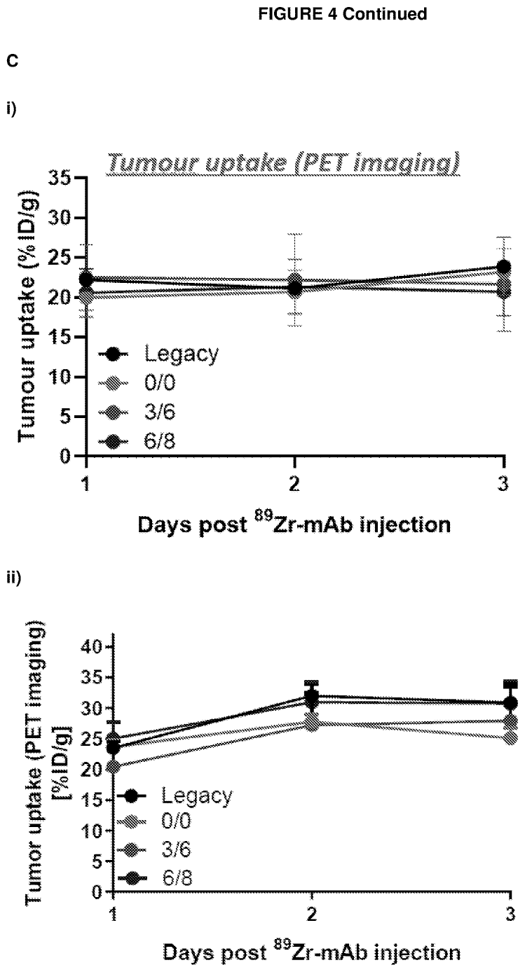

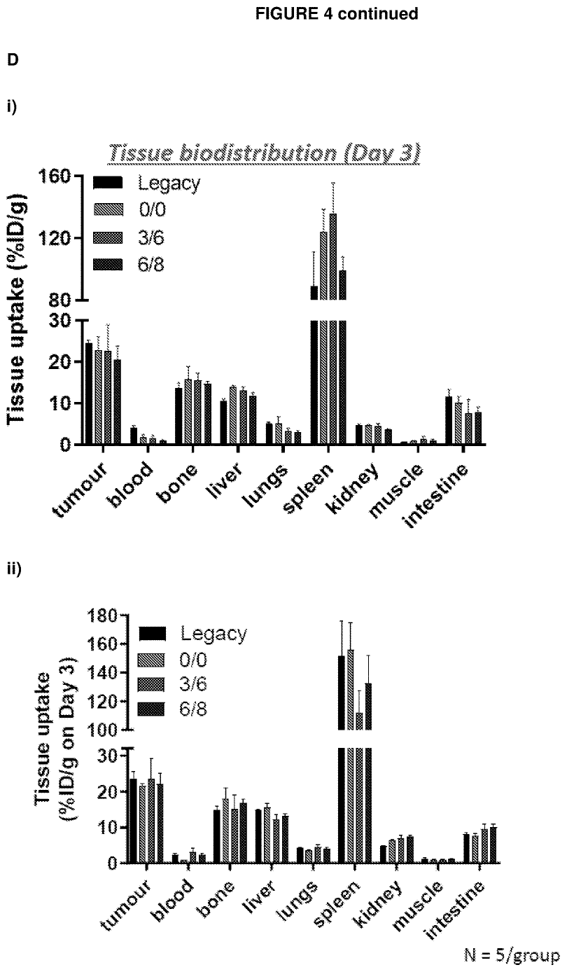

A summary of the experimental outline is provided in FIG. 4A. An exemplary PET scan (day 3) is provided in FIG. 4B with the location of the tumour shown with an arrow.

The results indicate that there is no significant difference in uptake into tumour or healthy tissue between the humanised variants compared to chimeric parental antibody.

The tumour uptake over time was determined via PET imaging. No significant differences between antibodies in tumour uptake were observed. (FIG. 4C).

Tissue biodistribution at the completion of the study (day 3) also indicates that there were no significant differences between the antibodies in terms of tumour uptake. (FIG. 4D). (The mice used in the study were NSG mice, which have miniature, defective spleens. As such, the high splenic uptake observed is expected in these mice and is not expected to represent uptake of a “normal” spleen).

Similar experiments were completed using radiolabelled antibodies comprising the K322A mutation in the constant region of the antibody (FIGS. 4Cii and 4Dii), and using antibodies that did not comprise this mutation (FIGS. 4Ci and 4Di). The results indicate that the K322A mutation has no effect on biodistribution or tumour uptake of the antibodies.

In conclusion, the humanised antibodies behaved similarly to the chimeric APOMAB antibody in vivo. In particular the VH3/VK6 and VH6/VK8 antibodies are shown to both bind to dead cancer cells, particularly after chemotherapy and demonstrate better binding in vitro to target.

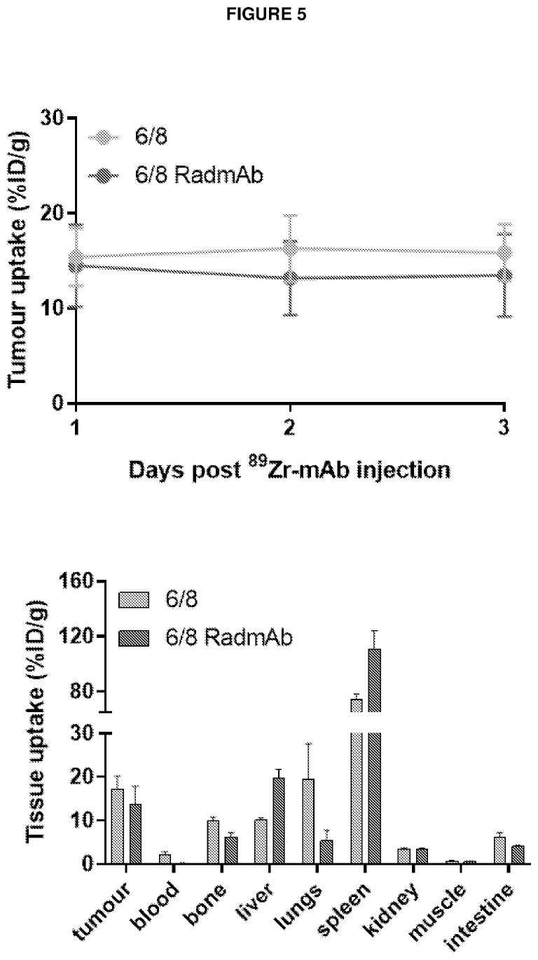

Results also indicate that in vivo distribution of the VH6/VK8 and VH6/VK8 RadMab antibodies is similar (FIG. 5).

1. An antigen binding protein for binding to La/SSB antigen, wherein the antigen binding protein comprises:
i. a CDR1, a CDR2 and a CDR3 of an antigen binding domain having a variable heavy chain as defined in SEQ ID NO: 3, and a CDR1, a CDR2 and a CDR3 of an antigen binding domain having a variable light chain as defined in SEQ ID NO: 6, wherein the CDRs are defined according to Kabat nomenclature;
ii. a CDR1, a CDR2 and a CDR3 of an antigen binding domain having a variable heavy chain as defined in SEQ ID NO: 1, and a CDR1, a CDR2 and a CDR3 of an antigen binding domain having a variable light chain as defined in SEQ ID NO: 5;
iii. a CDR1, a CDR2 and a CDR3 of an antigen binding domain having a variable heavy chain as defined in SEQ ID NO: 1, and a CDR1, a CDR2 and a CDR3 of an antigen binding domain having a variable light chain as defined in SEQ ID NO: 6, wherein the CDRs are defined accordingly to Kabat nomenclature;
iv. a CDR1, a CDR2 and a CDR3 of an antigen binding domain having a variable heavy chain as defined in SEQ ID NO: 2, and a CDR1, a CDR2 and a CDR3 of an antigen binding domain having a variable light chain as defined in SEQ ID NO: 4;
v. a CDR1, a CDR2 and a CDR3 of an antigen binding domain having a variable heavy chain as defined in SEQ ID NO: 2, and a CDR1, a CDR2 and a CDR3 of an antigen binding domain having a variable light chain as defined in SEQ ID NO: 5;
vi. a CDR1, a CDR2 and a CDR3 of an antigen binding domain having a variable heavy chain as defined in SEQ ID NO: 2, and a CDR1, a CDR2 and a CDR3 of an antigen binding domain having a variable light chain as defined in SEQ ID NO: 6;
vii. a CDR1, a CDR2 and a CDR3 of an antigen binding domain having a variable heavy chain as defined in SEQ ID NO: 3, and a CDR1, a CDR2 and a CDR3 of an antigen binding domain having a variable light chain as defined in SEQ ID NO: 4, wherein the CDRs are defined accordingly to Kabat nomenclature; or
viii. a CDR1, a CDR2 and a CDR3 of an antigen binding domain having a variable heavy chain as defined in SEQ ID NO: 3, and a CDR1, a CDR2 and a CDR3 of an antigen binding domain having a variable light chain as defined in SEQ ID NO: 5.

2. The antigen binding protein of claim 1, wherein:
a) when the antigen binding protein comprises i, above, the antigen binding protein further comprises a VH comprising a framework region (FR) 1 comprising an amino acid sequence of SEQ ID NO: 38, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 39, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 40, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 41, or a sequence at least about 80%, at least 85%, at least 90%, at least 91%, at least 92%, at least 93%, at least 94%, at least 95%, at least 96%, at least 97%, at least 98% or at least 99% thereto; and
a VL comprising a FR1 comprising an amino acid sequence of SEQ ID NO: 80, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 81, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 82, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 83, or a sequence at least about 80%, at least 85%, at least 90%, at least 91%, at least 92%, at least 93%, at least 94%, at least 95%, at least 96%, at least 97%, at least 98% or at least 99% thereto;
or
b) when the antigen binding domain comprises ii above, the antigen binding protein further comprises a VH comprising a framework region (FR) 1 comprising an amino acid sequence of SEQ ID NO: 10, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 11, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 12, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 13, or a sequence at least about 80%, at least 85%, at least 90%, at least 91%, at least 92%, at least 93%, at least 94%, at least 95%, at least 96%, at least 97%, at least 98% or at least 99% thereto; and
a VL comprising a FR1 comprising an amino acid sequence of SEQ ID NO: 66, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 67, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 68, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 69, or a sequence at least about 80%, at least 85%, at least 90%, at least 91%, at least 92%, at least 93%, at least 94%, at least 95%, at least 96%, at least 97%, at least 98% or at least 99% thereto;
or
c) when the antigen binding domain comprises iii above, the antigen binding protein further comprises a VH comprising a framework region (FR) 1 comprising an amino acid sequence of SEQ ID NO: 10, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 11, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 12, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 13, or a sequence at least about 80%, at least 85%, at least 90%, at least 91%, at least 92%, at least 93%, at least 94%, at least 95%, at least 96%, at least 97%, at least 98% or at least 99% thereto; and
a VL comprising a FR1 comprising an amino acid sequence of SEQ ID NO: 80, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 81, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 82, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 83, or a sequence at least about 80%, at least 85%, at least 90%, at least 91%, at least 92%, at least 93%, at least 94%, at least 95%, at least 96%, at least 97%, at least 98% or at least 99% thereto;
or
d) when the antigen binding domain comprises iv. above, the antigen binding protein further comprises a VH comprising a framework region (FR) 1 comprising an amino acid sequence of SEQ ID NO: 24, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 25, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 26, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 27, or a sequence at least about 80%, at least 85%, at least 90%, at least 91%, at least 92%, at least 93%, at least 94%, at least 95%, at least 96%, at least 97%, at least 98% or at least 99% thereto; and
a VL comprising a FR1 comprising an amino acid sequence of SEQ ID NO: 52, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 53, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 54, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 55, or a sequence at least about 80%, at least 85%, at least 90%, at least 91%, at least 92%, at least 93%, at least 94%, at least 95%, at least 96%, at least 97%, at least 98% or at least 99% thereto;
or
e) when the antigen binding domain comprises v, above, the antigen binding protein further comprises a VH comprising a framework region (FR) 1 comprising an amino acid sequence of SEQ ID NO: 24, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 25, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 26, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 27, or a sequence at least about 80%, at least 85%, at least 90%, at least 91%, at least 92%, at least 93%, at least 94%, at least 95%, at least 96%, at least 97%, at least 98% or at least 99% thereto; and
a VL comprising a FR1 comprising an amino acid sequence of SEQ ID NO: 66, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 67, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 68, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 69, or a sequence at least about 80%, at least 85%, at least 90%, at least 91%, at least 92%, at least 93%, at least 94%, at least 95%, at least 96%, at least 97%, at least 98% or at least 99% thereto;
or
f) when the antigen binding domain comprises vi. above, the antigen binding protein further comprises a VH comprising a framework region (FR) 1 comprising an amino acid sequence of SEQ ID NO: 24, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 25, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 26, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 27, or a sequence at least about 80%, at least 85%, at least 90%, at least 91%, at least 92%, at least 93%, at least 94%, at least 95%, at least 96%, at least 97%, at least 98% or at least 99% thereto; and
a VL comprising a FR1 comprising an amino acid sequence of SEQ ID NO: 80, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 81, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 82, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 83, or a sequence at least about 80%, at least 85%, at least 90%, at least 91%, at least 92%, at least 93%, at least 94%, at least 95%, at least 96%, at least 97%, at least 98% or at least 99% thereto;
or
g) when the antigen binding domain comprises vii. above, the antigen binding protein further comprises a VH comprising a framework region (FR) 1 comprising an amino acid sequence of SEQ ID NO: 38, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 39, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 40, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 41, or a sequence at least about 80%, at least 85%, at least 90%, at least 91%, at least 92%, at least 93%, at least 94%, at least 95%, at least 96%, at least 97%, at least 98% or at least 99% thereto; and
a VL comprising a FR1 comprising an amino acid sequence of SEQ ID NO: 52, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 53, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 54, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 55, or a sequence at least about 80%, at least 85%, at least 90%, at least 91%, at least 92%, at least 93%, at least 94%, at least 95%, at least 96%, at least 97%, at least 98% or at least 99% thereto;
or
h) when the antigen binding domain comprises viii above, the antigen binding protein further comprises a VH comprising a framework region (FR) 1 comprising an amino acid sequence of SEQ ID NO: 38, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 39, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 40, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 41, or a sequence at least about 80%, at least 85%, at least 90%, at least 91%, at least 92%, at least 93%, at least 94%, at least 95%, at least 96%, at least 97%, at least 98% or at least 99% thereto; and
a VL comprising a FR1 comprising an amino acid sequence of SEQ ID NO: 66, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 67, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 68, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 69, or a sequence at least about 80%, at least 85%, at least 90%, at least 91%, at least 92%, at least 93%, at least 94%, at least 95%, at least 96%, at least 97%, at least 98% or at least 99% thereto.

3. The antigen binding protein of claim 1 or 2, wherein the protein comprises a variable heavy chain comprising the amino acid sequence as set forth in SEQ ID NO: 3, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; and a variable light chain comprising the amino acid sequence as set forth in SEQ ID NO: 6; or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; wherein the variable heavy and/or light chains comprise no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19 or no more than 20 amino acid residue substitutions, deletions, or additions or combination thereof, wherein the substitutions, deletions, or additions or combination thereof are not in the CDRs, and wherein the antigen binding protein retains the ability to bind to La.

4. The antigen binding protein of claim 1 or 2, wherein the protein comprises a variable heavy chain comprising the amino acid sequence as set forth in SEQ ID NO: 1, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; and a variable light chain comprising the amino acid sequence as set forth in SEQ ID NO: 5; or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; wherein the variable heavy and/or light chains comprise no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19 or no more than 20 amino acid residue substitutions, deletions, or additions or combination thereof, wherein the substitutions, deletions, or additions or combination thereof are not in the CDRs, and wherein the antigen binding protein retains the ability to bind to La.

5. The antigen binding protein of claim 1 or 2, wherein the protein comprises a variable heavy chain comprising the amino acid sequence as set forth in SEQ ID NO: 1, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; and a variable light chain comprising the amino acid sequence as set forth in SEQ ID NO: 6; or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; wherein the variable heavy and/or light chains comprise no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19 or no more than 20 amino acid residue substitutions, deletions, or additions or combination thereof, wherein the substitutions, deletions, or additions or combination thereof are not in the CDRs defined according to Kabat, and wherein the antigen binding protein retains the ability to bind to La.

6. The antigen binding protein of claim 1 or 2, wherein the protein comprises a variable heavy chain comprising the amino acid sequence as set forth in SEQ ID NO: 2 or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; and a variable light chain comprising the amino acid sequence as set forth in SEQ ID NO: 4; or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; wherein the variable heavy and/or light chains comprise no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19 or no more than 20 amino acid residue substitutions, deletions, or additions or combination thereof, wherein the substitutions, deletions, or additions or combination thereof are not in the CDRs, and wherein the antigen binding protein retains the ability to bind to La.

7. The antigen binding protein of claim 1 or 2, wherein the protein comprises a variable heavy chain comprising the amino acid sequence as set forth in SEQ ID NO: 2, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; and a variable light chain comprising the amino acid sequence as set forth in SEQ ID NO: 5; or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; wherein the variable heavy and/or light chains comprise no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19 or no more than 20 amino acid residue substitutions, deletions, or additions or combination thereof, wherein the substitutions, deletions, or additions or combination thereof are not in the CDRs, and wherein the antigen binding protein retains the ability to bind to La.

8. The antigen binding protein of claim 1 or 2, wherein the protein comprises a variable heavy chain comprising the amino acid sequence as set forth in SEQ ID NO: 2, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; and a variable light chain comprising the amino acid sequence as set forth in SEQ ID NO: 6 or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; wherein the variable heavy and/or light chains comprise no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19 or no more than 20 amino acid residue substitutions, deletions, or additions or combination thereof, wherein the substitutions, deletions, or additions or combination thereof are not in the CDRs, and wherein the antigen binding protein retains the ability to bind to La.

9. The antigen binding protein of claim 1 or 2, wherein the protein comprises a variable heavy chain comprising the amino acid sequence as set forth in SEQ ID NO: 3, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; and a variable light chain comprising the amino acid sequence as set forth in SEQ ID NO: 4; or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; wherein the variable heavy and/or light chains comprise no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19 or no more than 20 amino acid residue substitutions, deletions, or additions or combination thereof, wherein the substitutions, deletions, or additions or combination thereof are not in the CDRs, and wherein the antigen binding protein retains the ability to bind to La.

10. The antigen binding protein of claim 1 or 2, wherein the protein comprises a variable heavy chain comprising the amino acid sequence as set forth in SEQ ID NO: 3, or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; and a variable light chain comprising the amino acid sequence as set forth in SEQ ID NO: 5; or a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical thereto; wherein the variable heavy and/or light chains comprise no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19 or no more than 20 amino acid residue substitutions, deletions, or additions or combination thereof, wherein the substitutions, deletions, or additions or combination thereof are not in the CDRs, wherein the CDRs are defined according to Kabat, and wherein the antigen binding protein retains the ability to bind to La.

11. The antigen binding protein of claim 1 or 2, wherein the protein comprises a VH comprising a complementarity determining region CDR1 comprising or consisting of an amino acid sequence of SEQ ID NO: 35; a CDR2 comprising or consisting of an amino acid sequence of SEQ ID NO: 36; a CDR3 comprising or consisting of an amino acid sequence of SEQ ID NO: 37; and
a VL comprising a complementarity determining region (CDR) 1 comprising or consisting of an amino acid sequence of SEQ ID NO: 77; a CDR2 comprising or consisting of an amino acid sequence of SEQ ID NO: 78; a CDR3 comprising or consisting of an amino acid sequence of SEQ ID NO: 79.

12. The antigen binding protein of claim 11, wherein the protein comprises a VH comprising a framework region (FR) 1 comprising an amino acid sequence of SEQ ID NO: 38, or a sequence at least about 87%, at least 88% at least 89%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 39, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 40, or a sequence at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 41; and
a VL comprising a FR1 comprising an amino acid sequence of SEQ ID NO: 80, or a sequence at least 70%, at least 71%, at least 72%, at least 73%, at least 74%, at least 75%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 81, or a sequence at least 94%, at least 95%, at least 96%, at least 97%, at least 98%, or at least 99% identical thereto; a FR3 comprising an amino acid sequence of SEQ ID NO: 82, or a sequence at least 94%, at least 95%, at least 96%, at least 97%, at least 98% or at least 99% identical thereto, and a FR4 comprising an amino acid sequence of SEQ ID NO: 83, or a sequence at least about 80%, at least 85%, at least 90%, at least 91%, at least 92%, at least 93%, at least 94%, at least 95%, at least 96%, at least 97%, at least 98% or at least 99% thereto.

13. The antigen binding protein of claim 1 or 2, wherein the protein comprises a VH comprising a complementarity determining region CDR1 comprising or consisting of an amino acid sequence of SEQ ID NO: 21; a CDR2 comprising or consisting of an amino acid sequence of SEQ ID NO: 22; a CDR3 comprising or consisting of an amino acid sequence of SEQ ID NO: 23; and
a VL comprising a complementarity determining region (CDR) 1 comprising or consisting of an amino acid sequence of SEQ ID NO: 63; a CDR2 comprising or consisting of an amino acid sequence of SEQ ID NO: 64; a CDR3 comprising or consisting of an amino acid sequence of SEQ ID NO: 65.

14. The antigen binding protein of claim 13, wherein the protein comprises a VH comprising a framework region (FR) 1 comprising an amino acid sequence of SEQ ID NO: 24, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 25, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 26, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 27, or a sequence at least about 80%, at least 85%, at least 90%, at least 91%, at least 92%, at least 93%, at least 94%, at least 95%, at least 96%, at least 97%, at least 98% or at least 99% thereto; and
a VL comprising a FR1 comprising an amino acid sequence of SEQ ID NO: 66, or a sequence at least about 80%, at least 85%, at least 90%, at least 91%, at least 92%, at least 93%, at least 94%, at least 95%, at least 96%, at least 97%, at least 98%, or at least 99% identical thereto; a FR2 comprising an amino acid sequence of SEQ ID NO: 67, or a sequence at least about 80%, at least 85%, at least 90%, at least 91%, at least 92%, at least 93%, at least 94%, at least 95%, at least 96%, at least 97%, at least 98%, or at least 99% identical thereto; a FR3 comprising an amino acid sequence of SEQ ID NO: 68, or a sequence at least about 80%, at least 85%, at least 90%, at least 91%, at least 92%, at least 93%, at least 94%, at least 95%, at least 96%, at least 97%, at least 98% or at least 99% identical thereto, and a FR4 comprising an amino acid sequence of SEQ ID NO: 69, or a sequence at least about 80%, at least 85%, at least 90%, at least 91%, at least 92%, at least 93%, at least 94%, at least 95%, at least 96%, at least 97%, at least 98% or at least 99% thereto.

15. The antigen binding protein of any one of claims 1 to 14, wherein the protein comprises:
an antigen binding domain having a variable heavy chain as defined in SEQ ID NO: 3, and an antigen binding domain having a variable light chain as defined in SEQ ID NO: 6;
an antigen binding domain having a variable heavy chain as defined in SEQ ID NO: 1, and an antigen binding domain having a variable light chain as defined in SEQ ID NO: 5;
an antigen binding domain having a variable heavy chain as defined in SEQ ID NO: 1, and an antigen binding domain having a variable light chain as defined in SEQ ID NO: 6;
an antigen binding domain having a variable heavy chain as defined in SEQ ID NO: 2, and an antigen binding domain having a variable light chain as defined in SEQ ID NO: 4;
an antigen binding domain having a variable heavy chain as defined in SEQ ID NO: 2, and an antigen binding domain having a variable light chain as defined in SEQ ID NO: 5;
an antigen binding domain having a variable heavy chain as defined in SEQ ID NO: 2, and an antigen binding domain having a variable light chain as defined in SEQ ID NO: 6;
an antigen binding domain having a variable heavy chain as defined in SEQ ID NO: 3, and an antigen binding domain having a variable light chain as defined in SEQ ID NO: 4; or
an antigen binding domain having a variable heavy chain as defined in SEQ ID NO: 3, and an antigen binding domain having a variable light chain as defined in SEQ ID NO: 5.

16. The antigen binding protein of any one of claims 1 to 15, wherein the antigen binding protein is in the form of:
(i) a single domain antibody (sdAb);
(ii) a single chain Fv fragment (scFv);
(iii) a dimeric scFv (di-scFv); or
(iv) one of (ii) or (iii) linked to a constant region of an antibody, Fc or a heavy chain constant domain (CH) 2 and/or CH3.

17. The antigen binding protein of any one of claims 1 to 15, wherein the protein is in the form of:
(i) a diabody;
(ii) a triabody;
(iii) a tetrabody;
(iv) a Fab;
(v) a F(ab′)2;
(vi) a Fv;
(vii) a bispecific antibody or other form of multispecific antibody; or
(viii) one of (i) to (vii) linked to a constant region of an antibody, Fc or a heavy chain constant domain (CH) 2 and/or CH3.

18. An immunoglobulin variable domain, antibody, dab, scFv, Fab, Fab′, F(ab′)2, Fv fragment, diabody, triabody, linear antibody, single-chain antibody molecule, or multispecific antibody comprising an antigen binding protein of any one of claims 1 to 15.

19. The antigen binding protein of any one of claims 1 to 15, wherein the protein is a naked antibody.

20. A fusion protein comprising an immunoglobulin variable domain, antibody, dab, scFv, Fab, Fab′, F(ab′)2, Fv fragment, diabody, triabody, linear antibody, single-chain antibody molecule, or multispecific antibody of claim 18.

21. A conjugate comprising an immunoglobulin variable domain, antibody, dab, scFv, Fab, Fab′, F(ab′)2, Fv fragment, diabody, triabody, linear antibody, single-chain antibody molecule, or multispecific antibody of claim 18 or a fusion protein of claim 20, conjugated to a label or a cytotoxic agent.

22. The conjugate of claim 21, wherein the cytotoxic agent is a chemotherapeutic agent or other agent used for the treatment of a disease, such as cancer.

23. The antigen binding protein of any one of claims 1 to 15, or the fusion protein of claim 20, or the conjugate of claim 21, comprising an Fc region that is engineered to:
increase the in vitro or in vivo half-life;
have an increased capacity to induce antibody-dependent cell mediated cytotoxicity (ADCC), antibody-dependent cellular phagocytosis (ADCP) or complement-dependent cytotoxicity;
reduce effector function; or
increase co-engagement of the protein or conjugate.

24. An nucleic acid encoding an antigen binding protein of any one of claims 1 to 15, or an, immunoglobulin variable domain, antibody, dab, scFv, Fab, Fab′, F(ab′)2, Fv fragment, diabody, triabody, linear antibody, single-chain antibody molecule, or multispecific antibody of claim 18 or a fusion protein of claim 20, wherein preferably, the nucleic acid has a nucleotide sequence that encodes any one or more of the amino acid sequences corresponding to SEQ ID NO: 1 to 6.

25. A vector comprising a nucleic acid of claim 24.

26. A cell comprising a nucleic acid of claim 24 or a vector of claim 25.

27. A pharmaceutical composition comprising an antigen binding protein of any one of claims 1 to 15, or an immunoglobulin variable domain, antibody, dab, scFv, Fab, Fab′, F(ab′)2, Fv fragment, diabody, triabody, linear antibody, single-chain antibody molecule, or multispecific antibody of claim 18, a fusion protein of claim 20, or a conjugate of claim 21, a nucleic acid of claim 24, a vector of claim 25, or a cell of claim 26, and a pharmaceutically acceptable carrier, diluent or excipient.

28. An immunoglobulin for binding to La/SSB antigen, wherein the immunoglobulin comprises an antigen binding protein of any one of claims 1 to 15.

29. The immunoglobulin of claim conjugated to a radionuclide.

30. The immunoglobulin of claim 29 wherein the radionuclides is selected from: actinium-225 (²²⁵Ac), astatine-211 (²¹¹At), bismuth-212 and bismuth-213 (²¹²Bi, ²¹³Bi), copper-64 and copper-67 (⁶⁴Cu, ⁶⁷Cu), gallium-67 and gallium-68 (⁶⁷Ga and ⁶⁸Ga), indium-111 (¹¹¹In), iodine-123, -124, -125 or -131 (¹²³I, ¹²⁴I, ¹²⁵I, ¹³¹I) (¹²³I), lead-212 (²¹²Pb), lutetium-177 (¹⁷⁷Lu), radium-223 (²²³Ra), samarium-153 (¹⁵³Sm), scandium-44 and scandium-47 (⁴⁴Sc, ⁴⁷Sc), strontium-90 (⁹⁰Sr), technetium-99 (^{99m}Tc), yttrium-86 and yttrium-90 (⁸⁶Y, ⁹⁰Y), zirconium-89 (⁸⁹Zr).

31. The immunoglobulin of claim 30, wherein the immunoglobulin is conjugated to the radionuclide directly (such as by halogenation of amino acid residues).

32. The immunoglobulin of claim 30, wherein the immunoglobulin is conjugated to the radionuclide by way of a linker or chelator moiety.

33. The immunoglobulin of claim 32, wherein the immunoglobulin is conjugated to a chelating moiety, selected from the group consisting of: TMT (6,6″-bis[N,N″,N″-tetra(carboxymethyl)aminomethyl)-4′-(3-amino-4-methoxyphenyl)-2,2′:6′,2″-terpyridine), DOTA (1,4,7,10-tetraazacyclododecane-NN′,N″(N′″-tetraacetic acid), TCMC, DO3A, CB-DO2A, NOTA, Diamsar, DTPA, CHX-A″-DTPA, TETE, Te2A, HBED, DFO, DFOsq, DFO-NCS and HOPO a chelator as described in WO 2022/133537 or other chelating agent.

34. The immunoglobulin of claim 32, wherein the immunoglobulin is conjugated to a bifunctional linker, for example, bromoacetyl, thiols, succinimide ester, TFP ester, a maleimide, or using any amine or thiol-modifying chemistry known in the art.

35. The immunoglobulin of any one of claims 28 to 34, comprising one or more amino acid substitutions in the Fc region of the immunoglobulin for reducing affinity of the immunoglobulin for the neonatal Fc receptor (FcRN), thereby reducing the serum half-life of the immunoglobulin compared to an immunoglobulin that does not comprise the amino acid substitutions (or compared to a wild-type immunoglobulin).

36. The immunoglobulin of claim 35, wherein the one or more amino acid substitutions is a substitution in the heavy chain constant region 2 (CH2) of immunoglobulin, or is a substitution in the heavy chain constant region 3 (CH3) of the immunoglobulin, or a combination thereof.

37. The immunoglobulin of claim 35 or 36, wherein the one or more amino acid substitutions is at one or more of residues His310, His433, His435, His436, or Ile253 of the Fc region of an immunoglobulin (numbering per Kabat nomenclature).

38. The immunoglobulin of claim 37, wherein the amino acid substitutions comprise a substitution in the heavy chain constant region at positions His310 and/or at His435, preferably, at both His310 and His435.

39. The immunoglobulin of any one of claims 35 to 38 wherein the immunoglobulin retains the ability to bind to one or more Fc-gamma receptors.

40. A method of determining the efficacy of a cytotoxic treatment, the method comprising:
administering an antigen binding protein of any one of claims 1 to 15 or an immunoglobulin of any one of claims 28 to 39 to a subject who has received a treatment with a cytotoxic agent, wherein the antigen binding protein or immunoglobulin comprises a detectable moiety for enabling detection thereof,
detecting the antigen binding protein or immunoglobulin in the subject,
determining that the treatment has been effective to cause cell death when the detection of the antigen binding protein or the immunoglobulin in the subject is above a background level;
determining that the treatment has not been effective to cause cell death when the detection of the antigen binding protein or the immunoglobulin in the subject is at the same or at a lower level compared to a background level
thereby determining the efficacy of the cytotoxic treatment.

41. The method of claim 40, wherein the background or standard level is obtained from the subject prior to the treatment with the cytotoxic agent.

42. A method of determining the efficacy of a cytotoxic treatment, the method comprising:
administering an antigen binding protein of any one of claims 1 to 15 or an immunoglobulin of any one of claims 28 to 39 to a subject who has received a treatment with a cytotoxic agent, wherein the antigen binding protein or the immunoglobulin comprises a detectable moiety for enabling detection thereof,
detecting the antigen binding protein or the immunoglobulin in the subject,
determining that the treatment has been effective to cause cell death based on the output of a machine learning model or algorithm indicating the presence or increased amount of La antigen in the subject;
determining that the treatment has not been effective to cause cell death based on the output of a machine learning model or algorithm indicating an absence of La antigen in the subject,
thereby determining the efficacy of the cytotoxic treatment.

44. The method of claim 42, wherein the machine learning model or algorithm is derived from data obtained from the subject prior to the treatment with the cytotoxic agent.

45. The method of any one of claims 40 to 44, wherein when the treatment is determined to be effective, the method comprises continuing to administer the cytotoxic agent.

46. The method of any one of claims 40 to 44, wherein when the treatment is determined not to have been effective, the method comprises discontinuation of the treatment, administration of a higher dose of the cytotoxic agent, or supplementation with additional cytotoxic treatments.

47. A method of treating a subject with a cytotoxic agent, the method comprising:
administering a cytotoxic agent to a subject, or providing a subject who has been administered a treatment with a cytotoxic agent,
administering an antigen binding protein as described herein to the subject, wherein the antigen binding protein comprises a detectable moiety for enabling detection thereof,
detecting the antigen binding protein in the subject,
determining to continue or continuing to administer the cytotoxic agent to the subject when the detection of the antigen binding protein in the subject is above a background level; or
determining to discontinue or discontinuing to administer the cytotoxic agent to the subject when the detection of the antigen binding protein in the subject is the same or lower than a background level, optionally determining to administer or administering an increased dose of the cytotoxic agent or an alternative or supplemental treatment to the subject.

48. The method of claim 47, wherein the background or standard level is obtained from the subject prior to the treatment with the cytotoxic agent.

49. A method of treating a subject with a cytotoxic agent, the method comprising:
administering a cytotoxic agent to a subject, or providing a subject who has been administered a treatment with a cytotoxic agent,
administering an antigen binding protein as described herein to the subject, wherein the antigen binding protein comprises a detectable moiety for enabling detection thereof,
detecting the antigen binding protein in the subject,
determining to continue or continuing to administer the cytotoxic agent to the subject based on the output of a machine learning model or algorithm indicating the presence or increased presence of La antigen in the subject;
determining to discontinue or discontinuing to administer the cytotoxic agent to the subject based on the output of a machine learning model or algorithm indicating an absence of La antigen in the subject, optionally determining to administer or administering an increased dose of the cytotoxic agent or an alternative or supplemental treatment to the subject.

50. The method of claim 49, wherein the machine learning model or algorithm is derived from data obtained from the subject prior to the treatment with the cytotoxic agent

51. The method of any one of claims 40 to 50, wherein the detectable moiety is any moiety which enables detection, preferably in vivo detection, of the antigen binding protein.

52. The method of claim 51, wherein the detectable moiety is a radionuclide or a fluorescent dye.

53. The method of claim 51, wherein the detectable moiety is a radionuclide selected from the group consisting of: fluorine-18 (¹⁸F), gallium-67 and gallium-68 (⁶⁷Ga and ⁶⁸Ga), indium-111 (¹¹¹In), iodine-123, -124, (¹²³I, ¹²⁴I) technetium-99 (^{99m}Tc), terbium-155 and terbium-159 (¹⁵⁵Tb and ¹⁵⁹Tb), and zirconium-89 (⁸⁹Zr).

54. The method of any one of claims 40 to 53, wherein the cytotoxic treatment or cytotoxic agent is selected from: chemotherapy, radiotherapy (such as external beam radiation therapy (EBRT), ultra-high dose rate (FLASH) radiotherapy, particle radiotherapy sometimes called hadrontherapy, with protons, neutrons or carbon ions), stereotactic ablative body radiotherapy (SABR), immunotherapy (such as immune checkpoint inhibition), allogenic or autologous stem cell transfer (SCT), chemo-immunotherapy or low-dose rate radiotherapy such as brachytherapy or molecular targeted radiation such as radioimmunotherapy or peptide receptor radionuclide therapy.

55. The method of any one of claims 40 to 53, wherein the cytotoxic treatment is for the treatment of cancer.

56. Use of an antigen binding protein of any one of claims 1 to 15, in the manufacture of a medicament for:
determining the efficacy of a cytotoxic treatment; or
treating a subject with a cytotoxic agent;
wherein the antigen binding protein comprising a detectable moiety for enabling detection thereof.

57. The use of claim 56, wherein the medicament is for use in a method that comprises:
administering an antigen binding protein of any one of claims 1 to 15 to the subject, wherein the antigen binding protein comprises a detectable moiety for enabling detection thereof,
detecting the antigen binding protein in the subject,
determining that the treatment has been effective when the detection of the antigen binding protein in the subject is above a background level;
determining that the treatment has not been effective when the detection of the antigen binding protein in the subject is at the same or at a lower level compared to a background level; and optionally:
determining to continue or continuing to administer the cytotoxic agent to the subject when the detection of the antigen binding protein in the subject is above a background level; or
determining to discontinue or discontinuing to administer the cytotoxic agent to the subject when the detection of the antigen binding protein in the subject is the same or lower than a background level, optionally determining to administer or administering an increased dose of the cytotoxic agent or an alternative or supplemental treatment to the subject.

58. A method of treating, preventing or minimising progression of a disease or condition, preferably of cancer, in a subject, minimising, reducing or preventing growth of a tumor in a subject, minimising, reducing or preventing metastasis in a subject, or increasing survival of a subject, the method comprising:
providing a subject who has received a first cytotoxic therapy; and
administering to the subject, a second cytotoxic therapy in the form of an antigen binding protein as described herein, wherein the antigen binding protein is conjugated to, or associated with, a cytotoxic agent,
thereby treating, preventing or minimising progression of a disease or condition, preferably of cancer, in a subject; or minimising, reducing or preventing growth of a tumour in a subject, or minimising, reducing or preventing metastasis in a subject, or increasing survival of a subject.

59. A combination therapy for treating a disease or condition, preferably cancer, the method comprising:
administering to a subject in need thereof, a first cytotoxic therapy; and
administering to the subject, a second cytotoxic therapy in the form of an antigen binding protein of any one of claims 28 to 39, wherein the antigen binding protein is conjugated to, or associated with, a cytotoxic agent,
thereby treating a disease or condition, preferably cancer.

60. Use of an antigen binding protein of any one of claims 28 to 39, in the manufacture of a medicament for use as a second cytotoxic therapy for:
treating, preventing or minimising progression of a disease or condition, preferably of cancer, in a subject,
minimising, reducing or preventing growth of a tumor in a subject,
minimising, reducing or preventing metastasis in a subject, or
increasing survival of a subject,
wherein the antigen binding protein is conjugated to, or associates with, a cytotoxic agent and wherein the medicament is for administration to a subject that has received a first cytotoxic therapy.

61. Use of an antigen binding protein of any one of claims 1 to 15, in the manufacture of a medicament for use as a second cytotoxic therapy in combination with a first cytotoxic therapy, for:
treating, preventing or minimising progression of a disease or condition, preferably of cancer, in a subject,
minimising, reducing or preventing growth of a tumor in a subject,
minimising, reducing or preventing metastasis in a subject, or
increasing survival of a subject,
wherein the antigen binding protein is conjugated to, or associated with, a cytotoxic agent.

62. The method of claim 58 or combination therapy of claim 59, or use of claim 60 or 61, wherein the first cytotoxic therapy is selected from: chemotherapy, radiotherapy (such as external beam radiation therapy (EBRT), ultra-high dose rate (FLASH) radiotherapy, particle radiotherapy sometimes called hadrontherapy, with protons, neutrons or carbon ions), stereotactic ablative body radiotherapy (SABR), immunotherapy (such as immune checkpoint inhibition), allogenic or autologous stem cell transfer (SCT), chemo-immunotherapy or low-dose rate radiotherapy such as brachytherapy or molecular targeted radiation such as radioimmunotherapy or peptide receptor radionuclide therapy.

63. The method, combination therapy or use of any one of claims 59 to 62, wherein the cytotoxic agent conjugated to the antigen binding protein is a chemotherapeutic agent.

64. The method, combination therapy or use of any one of claims 59 to 62, wherein the cytotoxic agent conjugated to the antigen binding protein is a therapeutic radioisotope.

65. The method, combination therapy or use of claim 64, wherein the therapeutic radioisotope is an alpha-emitting, beta-emitting or a gamma-emitting radionuclide.

66. The method, combination therapy or us of claim 65 wherein the radioisotope is selected from actinium-225 (²²⁵Ac), astatine-211 (²¹¹At), bismuth-212 and bismuth-213 (²¹²Bi, ²¹³Bi), copper-64 and copper-67 (⁶⁴Cu, ⁶⁷Cu), fluorine-18 (¹⁸F), indium-111 (¹¹¹In), iodine-123, -124, -125 or -131 (¹²³I, ¹²⁴I, ¹²⁵I, ¹³¹I), lead-212 (²¹²Pb), lutetium-177 (¹⁷⁷Lu), radium-223 (²²³Ra), rhenium-188 (¹⁸⁸Re), samarium-153 (¹⁵³Sm), scandium-44 and scandium-47 (⁴⁴Sc, ⁴⁷Sc), strontium-90 (⁹⁰Sr), technetium-99 (^{99m}Tc), terbium-155 and terbium-159, yttrium-86 and yttrium-90 (⁸⁶Y, ⁹⁰Y) and zirconium-89 (⁸⁹Zr).

67. The method, combination therapy or use of any one of claims 59 to 66, wherein the first and second cytotoxic therapies are administered simultaneously.

68. The method or combination therapy of any one of claims 59 to 66, wherein the first and second cytotoxic therapies are administered separately or sequentially.

69. A method of treating, preventing or minimising progression of a disease or condition in a subject, minimising, reducing or preventing growth of a tumor in a subject, minimising, reducing or preventing metastasis in a subject, or increasing survival of a subject susceptible to or suffering from the disease or condition, the method comprising exposing the subject, or cells from the subject, to an antigen binding protein of any one of claims 1 to 15, wherein the disease or condition comprises a tumor and/or a metastasis which comprise cells expressing La/SSB antigen due to exposure to a first cytotoxic agent and the antigen binding protein is conjugated to, or associated with, a second cytotoxic agent.

70. The method according to claim 69, wherein the second cytotoxic agent is a therapeutic radioisotope.

71. Use of an antigen binding protein of any one of claims 1 to 15, in the manufacture of a medicament for
minimising, reducing or preventing growth of a tumor in a subject, and/or
minimising, reducing or preventing metastasis in a subject,
wherein the antigen binding protein is conjugated to, or associated with, a cytotoxic agent, and the aforementioned disease or condition, tumor or metastasis comprises cells expressing La/SSB antigen.

72. The use of claim 71, wherein the cytotoxic agent is a therapeutic radioisotope.


SEQUENCE LISTING (95 sequences)

SEQ ID NO: 1 - protein, 120 aa, synthetic construct
EVQLQQPGAELVKPGASVKLSCKASGYAFTHYYIYWIKQRPGQGLEWIGGVNPSNGGTHFNEKFKSKATLTVDKSSSTAYMQLSSLTSEDSAVYYCTRSEYDYGLGFAYWGQGTLLTVSS

SEQ ID NO: 2 - protein, 120 aa, synthetic construct
EVQLVQSGAEVKKPGASVKVSCKASGYAFTHYYIYWIRQAPGQGLEWMGGVNPSSGGTHYAEKFQGRVTLTVDTSSSTAYMELSSLRSEDTAVYYCTRSEYDYGLGFAYWGQGTLVTVSS

SEQ ID NO: 3 - protein, 120 aa, synthetic construct
EVQLVQSGAEVKKPGASVKVSCKASGYAFTHYYIYWIKQAPGQGLEWIGGVNPSNGGTHFNEKFKSRVTITVDKSASTAYMELSSLRSEDTAVYYCTRSEYDYGLGFAYWGQGTLVTVSS

SEQ ID NO: 4 - protein, 111 aa, synthetic construct
EIVMSQSPSSLAVSAGEKVTMSCKSSQSLLNSRTPKNYLAWYQQKPGQSPKLLIYWASTRKSGVPDRFTGSGSGTDFTLTISSVQAEDLAVYYCKQSYNLLTFGAGTKLEL

SEQ ID NO: 5 - protein, 112 aa, synthetic construct
DIVMTQSPLSLPVTPGEPASISCRSSQSLLSSRTPKNYLAWYQQKPGQSPQLLIYWASTRKSGVPDRFSGSGSGTDFTLKISRVEAEDVGVYYCKQSYNLLTFGQGTKLEIK

SEQ ID NO: 6 - protein, 112 aa, synthetic construct
EIVMTQSPDSLAVSLGERATINCKSSQSLLNSRTPKNYLAWYQQKPGQPPKLLIYWASTRESGVPDRFTGSGSGTDFTLTISSLQAEDVAVYYCKQSYNLLTFGQGTKLEIK

SEQ ID NO: 7 - protein, 5 aa, synthetic construct
HYYIY

SEQ ID NO: 8 - protein, 17 aa, synthetic construct
GVNPSNGGTHFNEKFKS

SEQ ID NO: 9 - protein, 11 aa, synthetic construct
SEYDYGLGFAY

SEQ ID NO: 10 - protein, 30 aa, synthetic construct
EVQLQQPGAELVKPGASVKLSCKASGYAFT

SEQ ID NO: 11 - protein, 14 aa, synthetic construct
WIKQRPGQGLEWIG

SEQ ID NO: 12 - protein, 32 aa, synthetic construct
KATLTVDKSSSTAYMQLSSLTSEDSAVYYCTR

SEQ ID NO: 13 - protein, 11 aa, synthetic construct
WGQGTLLTVSS

SEQ ID NO: 14 - protein, 8 aa, synthetic construct
GYAFTHYY

SEQ ID NO: 15 - protein, 8 aa, synthetic construct
VNPSNGGT

SEQ ID NO: 16 - protein, 13 aa, synthetic construct
TRSEYDYGLGFAY

SEQ ID NO: 17 - protein, 25 aa, synthetic construct
EVQLQQPGAELVKPGASVKLSCKAS

SEQ ID NO: 18 - protein, 17 aa, synthetic construct
IYWIKQRPGQGLEWIGG

SEQ ID NO: 19 - protein, 38 aa, synthetic construct
HFNEKFKSKATLTVDKSSSTAYMQLSSLTSEDSAVYYC

SEQ ID NO: 20 - protein, 11 aa, synthetic construct
WGQGTLLTVSS

SEQ ID NO: 21 - protein, 5 aa, synthetic construct
HYYIY

SEQ ID NO: 22 - protein, 17 aa, synthetic construct
GVNPSSGGTHYAEKFQG

SEQ ID NO: 23 - protein, 11 aa, synthetic construct
SEYDYGLGFAY

SEQ ID NO: 24 - protein, 30 aa, synthetic construct
EVQLVQSGAEVKKPGASVKVSCKASGYAFT

SEQ ID NO: 25 - protein, 14 aa, synthetic construct
WIRQAPGQGLEWMG

SEQ ID NO: 26 - protein, 32 aa, synthetic construct
RVTLTVDTSSSTAYMELSSLRSEDTAVYYCTR

SEQ ID NO: 27 - protein, 11 aa, synthetic construct
WGQGTLVTVSS

SEQ ID NO: 28 - protein, 8 aa, synthetic construct
GYAFTHYY

SEQ ID NO: 29 - protein, 8 aa, synthetic construct
VNPSSGGT

SEQ ID NO: 30 - protein, 13 aa, synthetic construct
TRSEYDYGLGFAY

SEQ ID NO: 31 - protein, 25 aa, synthetic construct
EVQLVQSGAEVKKPGASVKVSCKAS

SEQ ID NO: 32 - protein, 17 aa, synthetic construct
IYWIRQAPGQGLEWMGG

SEQ ID NO: 33 - protein, 38 aa, synthetic construct
HYAEKFQGRVTLTVDTSSSTAYMELSSLRSEDTAVYYC

SEQ ID NO: 34 - protein, 11 aa, synthetic construct
WGQGTLVTVSS

SEQ ID NO: 35 - protein, 5 aa, synthetic construct
HYYIY

SEQ ID NO: 36 - protein, 17 aa, synthetic construct
GVNPSNGGTHFNEKFKS

SEQ ID NO: 37 - protein, 11 aa, synthetic construct
SEYDYGLGFAY

SEQ ID NO: 38 - protein, 30 aa, synthetic construct; identical to SEQ ID NO: 24

SEQ ID NO: 39 - protein, 14 aa, synthetic construct
WIKQAPGQGLEWIG

SEQ ID NO: 40 - protein, 32 aa, synthetic construct
RVTITVDKSASTAYMELSSLRSEDTAVYYCTR

SEQ ID NO: 41 - protein, 11 aa, synthetic construct
WGQGTLVTVSS

SEQ ID NO: 42 - protein, 8 aa, synthetic construct
GYAFTHYY

SEQ ID NO: 43 - protein, 8 aa, synthetic construct
VNPSNGGT

SEQ ID NO: 44 - protein, 13 aa, synthetic construct
TRSEYDYGLGFAY

SEQ ID NO: 45 - protein, 25 aa, synthetic construct
EVQLVQSGAEVKKPGASVKVSCKAS

SEQ ID NO: 46 - protein, 17 aa, synthetic construct
IYWIKQAPGQGLEWIGG

SEQ ID NO: 47 - protein, 38 aa, synthetic construct
HFNEKFKSRVTITVDKSASTAYMELSSLRSEDTAVYYC

SEQ ID NO: 48 - protein, 11 aa, synthetic construct
WGQGTLVTVSS

SEQ ID NO: 49 - protein, 17 aa, synthetic construct
KSSQSLLNSRTPKNYLA

SEQ ID NO: 50 - protein, 7 aa, synthetic construct
WASTRKS

SEQ ID NO: 51 - protein, 8 aa, synthetic construct
KQSYNLLT

SEQ ID NO: 52 - protein, 23 aa, synthetic construct
EIVMSQSPSSLAVSAGEKVTMSC

SEQ ID NO: 53 - protein, 15 aa, synthetic construct
WYQQKPGQSPKLLIY

SEQ ID NO: 54 - protein, 32 aa, synthetic construct
GVPDRFTGSGSGTDFTLTISSVQAEDLAVYYC

SEQ ID NO: 55 - protein, 10 aa, synthetic construct
FGAGTKLELK

SEQ ID NO: 56 - protein, 12 aa, synthetic construct
QSLLNSRTPKNY

SEQ ID NO: 57 - skipped

SEQ ID NO: 58 - protein, 8 aa, synthetic construct
KQSYNLLT

SEQ ID NO: 59 - protein, 26 aa, synthetic construct
EIVMSQSPSSLAVSAGEKVTMSCKSS

SEQ ID NO: 60 - protein, 17 aa, synthetic construct
LAWYQQKPGQSPKLLIY

SEQ ID NO: 61 - protein, 36 aa, synthetic construct
TRKSGVPDRFTGSGSGTDFTLTISSVQAEDLAVYYC

SEQ ID NO: 62 - protein, 10 aa, synthetic construct
FGAGTKLELK

SEQ ID NO: 63 - protein, 17 aa, synthetic construct
RSSQSLLSSRTPKNYLA

SEQ ID NO: 64 - protein, 7 aa, synthetic construct
WASTRKS

SEQ ID NO: 65 - protein, 8 aa, synthetic construct
KQSYNLLT

SEQ ID NO: 66 - protein, 23 aa, synthetic construct
DIVMTQSPLSLPVTPGEPASISC

SEQ ID NO: 67 - protein, 15 aa, synthetic construct
WYQQKPGQSPQLLIY

SEQ ID NO: 68 - protein, 32 aa, synthetic construct
GVPDRFSGSGSGTDFTLKISRVEAEDVGVYYC

SEQ ID NO: 69 - protein, 10 aa, synthetic construct
FGQGTKLEIK

SEQ ID NO: 70 - protein, 12 aa, synthetic construct
QSLLSSRTPKNY

SEQ ID NO: 71 - skipped

SEQ ID NO: 72 - protein, 8 aa, synthetic construct
KQSYNLLT

SEQ ID NO: 73 - protein, 25 aa, synthetic construct
DIVMTQSPLSLPVTPGEPASISCRS

SEQ ID NO: 74 - protein, 17 aa, synthetic construct
LAWYQQKPGQSPQLLIY

SEQ ID NO: 75 - protein, 36 aa, synthetic construct
TRKSGVPDRFSGSGSGTDFTLKISRVEAEDVGVYYC

SEQ ID NO: 76 - protein, 10 aa, synthetic construct
FGQGTKLEIK

SEQ ID NO: 77 - protein, 17 aa, synthetic construct
KSSQSLLNSRTPKNYLA

SEQ ID NO: 78 - protein, 7 aa, synthetic construct
WASTRES

SEQ ID NO: 79 - protein, 8 aa, synthetic construct
KQSYNLLT

SEQ ID NO: 80 - protein, 23 aa, synthetic construct
EIVMTQSPDSLAVSLGERATINC

SEQ ID NO: 81 - protein, 15 aa, synthetic construct
WYQQKPGQPPKLLIY

SEQ ID NO: 82 - protein, 32 aa, synthetic construct
GVPDRFTGSGSGTDFTLTISSLQAEDVAVYYC

SEQ ID NO: 83 - protein, 10 aa, synthetic construct
FGQGTKLEIK

SEQ ID NO: 84 - protein, 12 aa, synthetic construct
QSLLNSRTPKNY

SEQ ID NO: 85 - skipped

SEQ ID NO: 86 - protein, 8 aa, synthetic construct
KQSYNLLT

SEQ ID NO: 87 - protein, 26 aa, synthetic construct
EIVMTQSPDSLAVSLGERATINCKSS

SEQ ID NO: 88 - protein, 17 aa, synthetic construct
LAWYQQKPGQPPKLLIY

SEQ ID NO: 89 - protein, 36 aa, synthetic construct
TRESGVPDRFTGSGSGTDFTLTISSLQAEDVAVYYC

SEQ ID NO: 90 - protein, 10 aa, synthetic construct
FGQGTKLEIK

SEQ ID NO: 91 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLAQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPGK

SEQ ID NO: 92 - protein, 450 aa, synthetic construct
EVQLVQSGAEVKKPGASVKVSCKASGYAFTHYYIYWIRQAPGQGLEWMGGVNPSSGGTHYAEKFQGRVTLTVDTSSSTAYMELSSLRSEDTAVYYCTRSEYDYGLGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLAQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPGK

SEQ ID NO: 93 - protein, 450 aa, synthetic construct
EVQLVQSGAEVKKPGASVKVSCKASGYAFTHYYIYWIKQAPGQGLEWIGGVNPSNGGTHFNEKFKSRVTITVDKSASTAYMELSSLRSEDTAVYYCTRSEYDYGLGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLAQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPGK

SEQ ID NO: 94 - protein, 219 aa, synthetic construct
DIVMTQSPLSLPVTPGEPASISCRSSQSLLSSRTPKNYLAWYQQKPGQSPQLLIYWASTRKSGVPDRFSGSGSGTDFTLKISRVEAEDVGVYYCKQSYNLLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 95 - protein, 219 aa, synthetic construct
EIVMTQSPDSLAVSLGERATINCKSSQSLLNSRTPKNYLAWYQQKPGQPPKLLIYWASTRESGVPDRFTGSGSGTDFTLTISSLQAEDVAVYYCKQSYNLLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC